(19) United States
(12) Patent Application Publication
(10) Pub. No.: US 2026/0294946 A1
(43) Pub. Date: Oct. 1, 2026

(54) METHOD TO REDUCE FEED CONVERSION RATIO IN A FARMED ANIMAL

(71) Applicant: Inbiose N.V., Zwijnaarde (BE)

(72) Inventors: Joeri Beauprez, Zwijnaarde (BE); Ut Van Nguyen, Zwijnaarde (BE); Annelies Vercauteren, Zwijnaarde (BE); Mariska Van De Vosse, Voorthuizen (NL); Jordi Lensink, Voorthuizen (NL)

(21) Appl. No.: 19/480,639

(22) PCT Filed: May 3, 2024
(86) PCT No.: PCT/EP2024/062257

§ 371 Date: Oct. 31, 2025

(30) Foreign Application Priority Data
May 4, 2023 (EP) 23171609.3
May 4, 2023 (LU) LU504125
Jun. 14, 2023 (EP) 23179352.2
Jul. 14, 2023 (EP) 23185632.9

(51) Int. Cl. A61K 31/702; A61P 3/00

(52) U.S. Cl. CPC A61K 31/702; A61P 3/00

(57) ABSTRACT

The present disclosure relates to a method to reduce the feed conversion ratio (FCR) of a farm animal, wherein the method comprises the step of administering (i) a fucosylated mammalian milk oligosaccharide, (ii) a sialylated mammalian milk oligosaccharide, or (iii) a fucosylated mammalian milk oligosaccharide and a sialylated mammalian milk oligosaccharide. It further relates to the use of (i) a fucosylated mammalian milk oligosaccharide, (ii) a sialylated mammalian milk oligosaccharide, or (iii) a fucosylated mammalian milk oligosaccharide and a sialylated mammalian milk oligosaccharide to reduce the FCR of a farmed animal. It also relates to a method to increase the body weight of a farmed animal, wherein the method comprises the step of administering a fucosylated mammalian milk oligosaccharide and a sialylated mammalian milk oligosaccharide.

### CROSS-REFERENCE TO RELATED APPLICATIONS

This application is a national phase entry under 35 U.S.C. § 371 of International Patent Application PCT/EP2024/062257, filed May 3, 2024, designating the United States of America and published as International Patent Publication WO 2024/227919 A1 on Nov. 7, 2024, which claims the benefit under Article 8 of the Patent Cooperation Treaty of European Patent Application Serial No. 23171609.3 filed May 4, 2023, Luxembourg Patent Application Serial No. LU504125 filed May 4, 2023, European Patent Application Serial No. 23179352.2 filed Jun. 14, 2023, and European Patent Application Serial No. 23185632.9 filed Jul. 14, 2023.

### TECHNICAL FIELD

The disclosure relates to a method to reduce the feed conversion ratio (FCR) of a farm animal.

### BACKGROUND

Feed for a farm animal such as cattle, pigs and poultry, constitutes more than 60% of the total farm expenses. Hence, increasing the feed efficiency has a strong influence on financial returns for farmers. Furthermore, in a world with a growing population eating more animal protein, it is of paramount importance to improve the growth performance of farm animals.

### BRIEF SUMMARY

It was surprisingly found that a fucosylated mammalian milk oligosaccharide (MMO) and/or a sialylated MMO can decrease the feed conversion ratio (FCR) of a farm animal. In particular, the combination of a fucosylated MMO and a sialylated MMO results in an unexpected and strong reduction of the FCR of a farm animal. The fucosylated MMO and the sialylated MMO are not associated with adverse effects and are safe to use in farm animals.

Hence, the first aspect of the disclosure provides a method to reduce the feed conversion ratio (FCR) of a farm animal, wherein the method comprises the step of administering (i) a fucosylated MMO, (ii) a sialylated MMO, or (iii) a fucosylated MMO and a sialylated MMO. The second aspect of the disclosure provides the use of (i) a fucosylated MMO, (ii) a sialylated MMO, or (iii) a fucosylated MMO and a sialylated MMO to reduce the FCR of a farm animal. Finally, the third aspect of the disclosure provides a method to increase the body weight of a farm animal, wherein the method comprises the step of administering a fucosylated MMO and a sialylated MMO.

### DETAILED DESCRIPTION

### Method to Reduce Feed Conversion Ratio

In a first aspect, the disclosure provides a method to reduce the feed conversion ratio (FCR) of a farm animal, wherein the method comprises the step of administering to the farm animal: (i) a fucosylated mammalian milk oligosaccharide (MMO), (ii) a sialylated MMO, or (iii) a fucosylated MMO and a sialylated MMO.

In other words, the disclosure provides a method to reduce the feed conversion ratio (FCR) of a farm animal, wherein the method comprises the step of administering to the farm animal: (i) a fucosylated mammalian milk oligosaccharide (MMO), (ii) a sialylated MMO, or (iii) a fucosylated MMO and a sialylated MMO; and wherein the (i), (ii) or (iii) reduces the FCR of the farm animal.

Yet in other words, the disclosure provides a method of feeding a farm animal, wherein the method comprises the step of administering to the farm animal: (i) a fucosylated mammalian milk oligosaccharide (MMO), (ii) a sialylated MMO, or (iii) a fucosylated MMO and a sialylated MMO; and wherein the (i), (ii) or (iii) reduces the FCR of the farm animal.

Surprisingly, it was found that a method according to the disclosure significantly reduces the FCR of a farm animal, even when fed ad libitum.

In the context of the disclosure, the term “feed conversion ratio (FCR)” is the ratio between the feed intake of a farm animal in a given period divided by its weight gain calculated over the same period. FCR is the inverse of “feed efficiency” (i.e., 1/FCR). Hence, throughout the application and claims, the expression “reduce the feed conversion ratio” can be interchangeably used with “increase the feed efficiency.” A farm animal has a lower FCR (and hence a higher feed efficiency) if the animal produces either a greater body mass with the same feed intake or the same body mass with less feed intake.

If, for example, a first farm animal consumes 200 kg feed over a period of 12 weeks and the animal gained 87 kg weight over the same period, then the feed conversion ratio of the first farm animal is 87 kg divided by 200 kg, i.e., FCR is 2.30. When a second farm animal consumes 202 kg feed over the period of 12 weeks and the second farm animal has a FCR of 2.17, which is hence 5.7% animal gained 93 kg, then the second farm animal has a FCR of 2.17, which is hence 5.7% lower than that of the first farm animal.

The body weight of a farm animal can be readily assessed by an appropriate and dedicated scale, depending on the nature of the farm animal. As assessing the weight of a farm animal is imperative for animal performance, tracking estimated profits and calculating feed efficiency, all commercial farms have agricultural scales readily available and in use. Examples for large animals (e.g., cattle, horse, sheep) include platform/floor scales and pen-type scales (single animal scales). For smaller animals (e.g., calves, lambs, goats) farm bar scales are suitable. For poultry, bench scales (weighing a single bird), floor/platform scales and hanging scales (weighing a group of birds) are available.

The feed intake of a farm animal can also be readily assessed by the skilled person. In one case, the animal is individually housed. Here, it is not necessary to have means for identifying the animal, it is sufficient to detect the disappearance of feed. In another case, a group of animals is housed together. Here, it is a prerequisite that each animal can be individually identified so that the disappearance of feed can be directly linked to the correct animal. Systems for assessing the feed intake of a farm animal are readily available: for example, cattle (Bloch et al., 2021, Animal 15(7); Calan gate; GrowSafe Feed Intake and Behavioural Measurement System), pigs (Maselyne et al., 2015, Physiol. Behav. 138: p. 37-51), poultry (Bird Individual Ration Dispenser-electronic developed by INRAE and ITAVI), fish (Helland et al., 1996, Aquaculture 139(1-2): p. 157-163; Jobling et al., 2006, aquaculture Nutrition 1(3): p. 131-143).

In the context of the disclosure, any known method to assess FCR can be suitably used. While not a prerequisite, it is preferred that the body weight and feed intake of the farm animal is assessed using the same methods on the first day and last day of the period.

In a preferred embodiment of a method according to the disclosure, the FCR is calculated over a given period, wherein the period is 2 weeks, more preferably 3 weeks, even more preferably 4 weeks, even more preferably 5 weeks, even more preferably 6 weeks, even more preferably 8 weeks, even more preferably 10 weeks, most preferably 12 weeks.

In an additional and/or preferred alternative embodiment, the FCR is calculated over a period that corresponds with 25.0%, preferably 50.0%, more preferably 75.0, most preferably 100.0%, of the total period wherein the oligosaccharide(s) according to the disclosure is/are administered to the farm animal (it is in this context referred to in the section titled “Method to reduce FCR”). For example, if a farm animal receives a fucosylated MMO according to the disclosure for a period of 12 weeks, then the FCR is preferably calculated over a period of 3 weeks (i.e., 25.0%), more preferably 6 weeks (i.e., 50.0%), even more preferably 9 weeks (i.e., 75%) and most preferably 12 weeks (i.e., 100.0%).

In an additional and/or preferred alternative embodiment, the method according to the disclosure reduces the FCR of a farm animal with at least 1.0%, 2.0%, 3.0%, 4.0%, 5.0%, 6.0%, 7.0%, 8.0%, 9.0% or 10.0%, preferably relative to the FCR of the animal prior to administration of i) a fucosylated MMO, (ii) a sialylated MMO or (iii) a fucosylated MMO and a sialylated MMO according to the disclosure. Preferably, the FCR reduces with at least 2.0%, more preferably at least 2.5%, even more preferably at least 3.0%, even more preferably at least 3.5%, even more preferably at least 4.0%, even more preferably at least 4.5%, even more preferably at least 5.0%, most preferably at least 7.0%. Throughout the application and claims, the term “reduces the FCR” can be replaced with “increases the feeding efficiency” and vice versa.

For example, if a farm animal has a FCR of 2.30 just before the onset of administering i) a fucosylated MMO, (ii) a sialylated MMO or (iii) a fucosylated MMO and a sialylated MMO according to the disclosure, and the FCR on the last day of administration is 2.17 (for example, after 12 weeks), then it can be concluded that the method according to the disclosure has reduced the FCR of the farm animal with 5.7% after a treatment lasting 12 weeks.

In an additional and/or preferred alternative embodiment, the method according to the disclosure reduces the FCR of a farm animal with at least 1.0%, 2.0%, 3.0%, 4.0%, 5.0%, 6.0%, 7.0%, 8.0%, 9.0% or 10.0%, preferably relative to the FCR of a control farm animal that did not receive the administration of i) a fucosylated MMO, (ii) a sialylated MMO or (iii) a fucosylated MMO and a sialylated MMO according to the disclosure. Preferably, the FCR reduces with at least 2.0%, more preferably at least 2.5%, even more preferably at least 3.0%, even more preferably at least 3.5%, even more preferably at least 4.0%, even more preferably at least 4.5%, even more preferably at least 5.0%, most preferably at least 7.0%.

In a more preferred embodiment, the FCR is calculated over a period that corresponds with:
if subject is bovine (Manafiazar et al., 2017, Can. J. Anim. Sci. 97: p. 456-465): 6 weeks, preferably 8 weeks, more preferably 10 weeks, most preferably 12 weeks;
if subject is a pig: 3 weeks, preferably 4 weeks, more preferably 5 weeks, even more preferably 6 weeks, even more preferably 8 weeks, even more preferably 10 weeks, most preferably 12 weeks;
if subject is a sheep (Tortereau et al., 2020, Animal 14(4): p. 681-687): 6 weeks, preferably 8 weeks, more preferably 10 weeks, most preferably 12 weeks;
if subject is a goat: 6 weeks, preferably 8 weeks, more preferably 10 weeks, most preferably 12 weeks;
if subject is a horse: 6 weeks, preferably 8 weeks, more preferably 10 weeks, most preferably 12 weeks;
if subject is a camelid: 6 weeks, preferably 8 weeks, more preferably 10 weeks, most preferably 12 weeks;
if subject is a rabbit: 6 weeks, preferably 8 weeks, more preferably 10 weeks, most preferably 12 weeks;
if subject is a fish or a crustacean: 1 week, preferably 2 weeks, more preferably 3 weeks, even more preferably 4 weeks, even more preferably 5 weeks, even more preferably 6 weeks, even more preferably 8 weeks, even more preferably 10 weeks, most preferably 12 weeks;
if subject is poultry: 6 weeks, preferably 8 weeks, more preferably 10 weeks, most preferably 12 weeks.

The reduction of the feed conversion ratio in a farm animal by the method according to the disclosure is independent from the presence/absence and/or severity of diarrhea in the farm animal. In other words, a reduction of diarrhea in the farm animal is not a prerequisite to obtain a reduction of the feed conversion ratio by a method according to the disclosure.

Throughout the application and claims, each embodiment disclosed in the context wherein the subject is a horse, is also disclosed for a subject being a donkey, a mule or a hinny.

### Subject

In an embodiment of the first aspect of the disclosure, the method is to reduce the feed conversion ratio of a farm animal.

As understood by the skilled person, a “farm animal” refers to an animal that is reared in an agricultural setting in order to produce various commodities such as food (meat, organs, eggs, dairy products) and/or hair or wool. In the context of the disclosure, it is preferred that the farm animal is an animal that is reared in an agricultural setting in order to produce food, in particular, meat.

A farm animal is preferably selected from the group consisting of bovine, pig, sheep, goat, horse, donkey, mule, hinny, camelid (preferably llama or alpaca), rabbit, fish, crustacean and poultry; more preferably selected from the group consisting of bovine, pig, sheep, goat, horse, camelid (preferably llama or alpaca), rabbit, fish, crustacean and poultry; more preferably selected from the group consisting of bovine, pig, sheep, goat, horse, camelid (preferably llama or alpaca), rabbit, fish and poultry; even more preferably selected from the group consisting of bovine, pig, sheep, goat, horse, camelid (preferably llama or alpaca) and poultry; even more preferably selected from the group consisting of bovine, pig, sheep, goat, camelid (preferably llama or alpaca) and poultry; even more preferably selected from the group consisting of bovine, pig, sheep, goat, and poultry; even more preferably selected from the group consisting of bovine, pig, sheep and goat; even more preferably selected from the group consisting of bovine, sheep and goat; and most preferably the farm animal is a bovine.

Alternatively, a farm animal is preferably selected from the group consisting of bovine and pig, most preferably the farm animal is a bovine.

In the context of the disclosure, the term “poultry” refers to birds bred by humans for their eggs, meat and/or feathers. Throughout the application and claims, the poultry preferably is a chicken, a quail, a turkey, a duck, a pigeon and a goose, more preferably a chicken, a quail or a turkey, more preferably chicken or a quail, most preferably a chicken. Throughout the application and claims, the term “chicken” is preferably replaced with broiler.

In a preferred embodiment, the animal is a multigastric animal, preferably a ruminant or camelid, more preferably a ruminant.

Preferably, the camelid is a llama or an alpaca, more preferably the camelid is an alpaca.

Preferably, the ruminant is selected from the group consisting of bovine, sheep and goat, more preferably the ruminant is bovine.

In a preferred alternative embodiment, the animal is a monogastric animal, preferably selected from the group consisting of pig, horse, rabbit, fish and poultry, more preferably selected from the group consisting of pig, horse, rabbit and poultry, even more preferably selected from the group consisting of pig, horse and poultry, even more preferably pig or horse, most preferably pig.

Throughout the description and claims, unless specifically stated otherwise, the terms “subject” and “animal” can be preferably replaced with “subject in the need thereof” and “animal in the need thereof,” respectively.

In a more preferred embodiment, the animal according to the disclosure is a non-adult animal. In an additional and/or a more preferred alternative embodiment, the animal according to the disclosure is a young animal. Throughout the application and claims, unless specifically stated otherwise, a “non-adult animal” can be preferably replaced with “juvenile animal” or “young animal.”

As understood by the skilled person:
a non-adult bovine is a calf,
a non-adult pig is a piglet;
a non-adult sheep is a lamb;
a non-adult goat is a kid;
a non-adult horse is a foal;
a non-adult llama is a cria;
a non-adult alpaca is a cria;
a non-adult rabbit is a kitten or a kit;
a non-adult fish is a fry (young fish that is capable of feeding himself)/fingerling (young fish that is capable of feeding himself and has developed scales and working fins, i.e., transition to a juvenile fish is complete), preferably a fingerling;
a non-adult poultry/chicken is a chick.

In an additional and/or more preferred alternative embodiment, the animal according to the disclosure is a preweaned animal.

In an additional and/or more preferred alternative embodiment, the animal according to the disclosure is an animal that is weaned.

As understood by the skilled person, weaning refers to the process of withdrawing the access of a young animal to milk and to gradually accustom the young animal to the adult diet.

### Oligosaccharide

In an embodiment of the first aspect of the disclosure, the method comprises the step of administering to the farm animal: (i) a fucosylated mammalian milk oligosaccharide (MMO), (ii) a sialylated MMO, or (iii) a fucosylated MMO and a sialylated MMO.

A “fucosylated mammalian milk oligosaccharide (MMO)” refers to a MMO that comprises a fucose. Preferably, the fucosylated MMO according to the disclosure contains only one fucose, i.e., the MMO consists of two or more monosaccharides and only one of the monosaccharides is a fucose.

A “sialylated mammalian milk oligosaccharide” refers to a MMO that comprises a sialic acid, preferably a N-acetylneuraminic acid (i.e., Neu5Ac). Preferably, the sialylated MMO according to the disclosure contains only one sialic acid (preferably only one Neu5Ac), i.e., the MMO consists of two or more monosaccharides and only one of the monosaccharides is a sialic acid (preferably a Neu5Ac).

As understood by the skilled person, mammalian milk oligosaccharides (MMOs) comprise oligosaccharides present in milk found in any phase during lactation including colostrum milk from humans (i.e., human milk oligosaccharides or HMOs) and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus) (Urashima T. et al., 2011, Milk Oligosaccharides, Nova Biomedical Books, New York ISBN 978-1-61122-831-1; Coppa et al., 2013, Ital. J. Pediatr. 2013, 39(2)). A replete amount of milk saccharide structures have been elucidated so far. The majority of milk oligosaccharides found in animals such as mammals and humans comprise lactose at the reducing end (Urashima et al., 2011). Other milk oligosaccharides comprise N-acetyllactosamine (Gal-β 1,4-GlcNAc) or lacto-N-biose (Gal-β1,3-GlcNAc) at the reducing end (Urashima et al., 2011; Wrigglesworth et al., 2020, PLoS ONE 15(12); Urashima et al., 2013, Biosci. Biotechnol. Biochem. 77(3): p. 455-466; Wei et al., 2018, Sci. Rep. 8:4688). Examples hereof are 3-FLN (Gal-β1,4-(Fuc-α1,3-)GlcNAc; also known as Lewis x antigen), 3′-SLN (Neu5Ac-α2,3-Gal-β1,4-GlcNAc), 6′-SLN (Neu5Ac-α2,6-Gal-β1,4-GlcNAc) (Urashima et al., 2011; Wrigglesworth et al., 2020; Wei et al., 2018). Further, milk saccharides comprise milk glycosaminoglycans (GAGs; Coppa et al., 2013; Rai et al., 2021, Int. J. Biol. Macromolecules, 193(A): p. 137-144). Antigens of the human ABO blood group system such as the A determinant (GalNAc-α1,3(Fuc-α1,2)-Gal), B determinant (Gal-α1,3(Fuc-α1,2)-Gal) and H-determinant (Fuc-α1,2-Gal) have been shown to be present in saccharides of the milk of mammals (e.g., a tetrasaccharide in a lion and leopard; Wrigglesworth et al., 2020).

Throughout the application and claims, a “mammalian milk oligosaccharide (MMO)” is preferably a “human milk oligosaccharide (HMO).”

In the context of the disclosure, the term “oligosaccharide” preferably refers to a saccharide containing 2 up to and including 20 monosaccharides, i.e., the degree of polymerization (DP) is 2-20, more preferably refers to a saccharide containing 3 up to and including 20 monosaccharides, i.e., the degree of polymerization (DP) is 3-20. In on other words, the term “oligosaccharide” preferably refers to a saccharide consisting of 2-20, more preferably 3-20, monosaccharide units that are linked to each other via glycosidic bonds in a linear or in a branched structure. The linkage (i.e., glycosidic linkage), between two sugar units, i.e., two monosaccharides, can be expressed, for example, as 1, 4, 1≥4, or (1-4), which are used interchangeably herein.

More preferably, an oligosaccharide as described herein consists of 3-8, even more preferably 3-7, even more preferably 3-6, most preferably 3-5, monosaccharides. For the sake of clarity, the expression “x-y” refers to a range from and including x to and including y. For example, 3-5 monosaccharides means that 3, 4 or 5 monosaccharides are present.

In a preferred embodiment of the disclosure, the method according to the disclosure comprises the step of administering to the farm animal a fucosylated mammalian milk oligosaccharide (MMO). It was surprisingly found in the disclosure that administering a fucosylated MMO, in particular, 2′-fucosyllactose (2′FL), to a farm animal results in a strong decrease of the feed conversion ratio of the animal.

In this context of the disclosure, it is a preferred embodiment that the fucosylated MMO is a neutral oligosaccharide. A “neutral” oligosaccharide as used herein and as generally understood in the state of the art is an oligosaccharide that has no negative charge originating from a carboxylic acid group.

In an additional and/or preferred alternative embodiment, the fucosylated MMO consists of 3-8, more preferably 3-7, even more preferably 3-6, most preferably 3-5, monosaccharides.

In an additional and/or preferred alternative embodiment, the fucosylated MMO comprises a fucose that is linked to a monosaccharide in an α-1,2-, α-1,3- or α-1,4-linkage, preferably an α-1,2- or an α-1,3-linkage, more preferably an α-1,2-linkage, and wherein the monosaccharide is preferably selected from glucose, N-acetylglucosamine and galactose, more preferably, the monosaccharide is glucose or galactose, even more preferably, the monosaccharide is galactose. The skilled person will understand that the expression “fucose is linked to a monosaccharide” refers to the situation wherein the fucose is bound to a monosaccharide through a glycosidic bond and wherein the fucose and monosaccharide are part of the oligosaccharide of the disclosure (which can comprise additional monosaccharide(s) than the fucose and the monosaccharide).

In an additional and/or preferred alternative embodiment, the fucosylated MMO comprises a lactose, a lacto-N-biose (LNB) or N-acetyllactosamine (LacNAc) at its reducing end, preferably, the fucosylated oligosaccharide comprises lactose or LacNAc at its reducing end, more preferably, the fucosylated oligosaccharide comprises lactose at its reducing end.

A fucosylated MMO comprising lactose at its reducing end is preferably selected form the group consisting of 2′-fucosyllactose (2′FL), 3-fucosyllactose (3-FL), difucosyllactose (diFL), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH), more preferably selected from the group consisting of 2′FL, diFL, LNFP I, GalNAc-LNFP I, Gal-LNFP I, LNDFH I, lewis b-lewis x, DFLNH (a), TFLNH and LNnFP I, even more preferably selected from the group consisting of 2′FL, diFL, LNFP I and LNnFP I, even more preferably, wherein the fucosylated MMO is 2′FL or diFL, most preferably, wherein the fucosylated MMO is 2′FL.

A fucosylated MMO comprising lacto-N-biose (LNB) at its reducing end is preferably selected from the group consisting of 2′-fucosyllacto-N-biose (2′FLNB), 4-fucosyllacto-N-biose (4FLNB) and difucosyllacto-N-biose (diFLNB), more preferably, wherein the fucosylated MMO is 2′FLNB or diFLNB, most preferably, wherein the fucosylated MMO is 2′FLNB.

A fucosylated MMO comprising N-acetyllactosamine (LacNac) at its reducing end is preferably selected from the group consisting of 2′-fucosyl-N-acetyllactosamine (2′FlacNAc), difucosyl-N-acetyllactosamine (diFLacNAc) and 3-fucosyl-N-acetyllactosamine (3FlacNAc), more preferably, wherein the fucosylated MMO is 2′FlacNAc or diFLacNAc, most preferably, wherein the fucosylated MMO is 2′FlacNAc.

In a more preferred embodiment, the fucosylated MMO according to the disclosure is selected from a group consisting of 2′-fucosyllactose (2′FL), 3-fucosyllactose (3-FL), difucosyllactose (diFL), 2′-fucosyl-N-acetyllactosamine (2′FlacNAc), difucosyl-N-acetyllactosamine (diFLacNAc), 3-fucosyl-N-acetyllactosamine (3FlacNAc), 2′-fucosyllacto-N-biose (2′FLNB), 4-fucosyllacto-N-biose (4FLNB), difucosyllacto-N-biose (diFLNB), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH). More preferably, the fucosylated MMO according to the disclosure is selected from the group consisting of 2′FL, diFL, LNFP I, GalNAc-LNFP I, Gal-LNFP I, LNDFH I, lewis b-lewis x, DFLNH (a), TFLNH, LNnFP I, 2′FLNB, diFLNB, 2′FlacNAc and diFLacNAc, even more preferably selected from the group consisting of 2′FL, diFL, LNFP I, LNnFP I, 2′FLNB, diFLNB, 2′FlacNAc and diFLacNAc, even more preferably selected from the group consisting of 2′FL, diFL, 2′FLNB, diFLNB, 2′FlacNAc and diFLacNAc, even more preferably selected from the group consisting of 2′FL, 2′FLNB and 2′FlacNAc, even more preferably, wherein the fucosylated MMO is 2′FL or 2′FLNB, most preferably, wherein the fucosylated MMO is 2′FL.

The method according to the disclosure does not exclude the administration of one or more additional MMOs, albeit it is not a prerequisite to achieve a reduction of the feed conversion ratio of a farm animal. In this regard, it is preferred that the method according to the disclosure comprises the administration of a fucosylated MMO as described herein and one or more sialylated MMOs, more preferably, a fucosylated MMO as described herein and two or more sialylated MMOs, even more preferably, a fucosylated MMO as described herein and two sialylated MMOs, as depicted further herein. In an alternative embodiment, the fucosylated MMO is the sole MMO administered to the farm animal according to the method of the disclosure.

Throughout the application and claims, unless specifically stated otherwise, “a fucosylated MMO” refers to at least one fucosylated MMO, preferably to one fucosylated MMO (i.e., no further fucosylated MMOs).

In an additional and/or preferred alternative embodiment of the disclosure, the method according to the disclosure comprises the step of administering to the farm animal a sialylated mammalian milk oligosaccharide (MMO), preferably two or more sialylated MMOs (i.e., a first sialylated MMO and a second sialylated MMO, optionally further sialylated MMOs are administered; preferably administered as a mixture), more preferably, two sialylated MMOs (i.e., a first sialylated MMO and a second sialylated MMO as described herein; preferably administered as a mixture). For the sake of clarity, the expression “more preferably, two sialylated MMOs,” in this context of the disclosure, means that no additional sialylated MMOs are administered to the subject. It was surprisingly found in the disclosure, that administering a sialylated MMO, in particular, the combination of 3′-sialyllactose (3′SL) and 6′-sialyllactose (6′SL), preferably no additional sialylated MMOs are administered, to a farm animal results in a strong decrease of the feed conversion ratio of the animal, albeit to a lesser extent than administering a fucosylated MMO as described earlier herein.

In other words, in an additional and/or preferred alternative embodiment, the method according to the disclosure comprises the step of administering to a farm animal:
(i) a fucosylated MMO;
(ii) a sialylated MMO, preferably two or more sialylated MMOs (i.e., a first sialylated MMO and a second sialylated MMO, optionally, further sialylated MMOs), more preferably, two sialylated MMO(s) (i.e., a first sialylated MMO and a second sialylated MMO); or
(iii) a fucosylated MMO and a sialylated MMO, preferably a fucosylated MMO and two or more sialylated MMOs (i.e., a first sialylated MMO and a second sialylated MMO, optionally, further sialylated MMOs), more preferably, a fucosylated MMO and two sialylated MMO(s) (i.e., a first sialylated MMO and a second sialylated MMO).

Preferably, the fucosylated MMO is as described earlier herein.

Hence, it is particularly preferred in the context of the disclosure that administering a sialylated MMO in a method according to the disclosure further comprises administering a second sialylated MMO (preferably no further sialylated MMOs).

In this context of the disclosure, it is a preferred embodiment that the sialylated MMO comprises a sialic acid, preferably a Neu5Ac, that is linked to a monosaccharide in an α-2,3-, α-2,6- or α-2,8-linkage, preferably an α-2,3- or an α-2,6-linkage, and wherein the monosaccharide is preferably selected from galactose, N-acetylglucosamine and Neu5Ac, more preferably, the monosaccharide is galactose or N-acetylglucosamine, even more preferably, the monosaccharide is galactose. The skilled person will understand that the expression “sialic acid is linked to a monosaccharide” refers to the situation wherein the sialic acid is bound to a monosaccharide through a glycosidic bond and wherein the sialic acid and monosaccharide are part of the oligosaccharide of the disclosure (which can comprise additional monosaccharide(s) than the sialic acid and the monosaccharide).

In an additional and/or preferred alternative embodiment, the sialylated MMO consists of 3-8, more preferably 3-7, even more preferably 3-6, most preferably 3-5 monosaccharides.

In an additional and/or preferred alternative embodiment, the sialylated MMO comprises a lactose, a lacto-N-biose (LNB) or N-acetyllactosamine (LacNAc) at its reducing end, preferably the sialylated MMO comprises lactose or LacNAc at its reducing end, most preferably the sialylated MMO comprises lactose at its reducing end.

A sialylated MMO comprising lactose at its reducing end is preferably selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c and LST d; more preferably selected from the group consisting of 3′SL, 6′SL, 3,6-disialyllactose, 6,6′-disialyllactose, 8,3-disialyllactose, LST a, LST b, LST c and LST d; even more preferably selected from the group consisting of 3′SL, 6′SL, LST a, LST b, LST c and LST d; most preferably wherein the sialylated MMO is 3′-sialyllactose (3′SL) or 6′-sialyllactose (6′SL).

A sialylated MMO comprising lacto-N-biose (LNB) at its reducing end is preferably selected form the group consisting of 3′-sialyllacto-N-biose (3′SLNB), 6′-sialyllacto-N-biose (6′SLNB) and monofucosylmonosialyllacto-N-octaose (sialyl Lewis a); more preferably, the sialylated MMO is 3′SLNB or 6′SLNB.

A sialylated MMO comprising N-acetyllactosamine (LacNac) at its reducing end is preferably selected form the group consisting of 3′-sialyllactosamine (3′SLacNAc), 6′-sialyllactosamine (6′SLacNAc) and sialyl Lewis x; more preferably, the sialylated MMO is 3′SLacNAc or 6′SLacNAc.

In a more preferred embodiment, the sialylated MMO according to the disclosure is selected from a group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x. More preferably, the sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably, the sialylated MMO is selected from the group consisting of 3′SL, 6′SL, 3′SLNB, 6′SLNB, 3′SLacNAc and 6′SLacNAc. Most preferably, the sialylated MMO is 3′-sialyllactose (3′SL) or 6′-sialyllactose (6′SL).

In an additional and/or more preferred alternative embodiment, the first sialylated MMO according to the disclosure is selected from a group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x. Even more preferably, the first sialylated MMO is selected from the group consisting of 3′SL, 6′SL, 3′S-2′FL, 6′S-2′FL, 3′S-3-FL, 6′S-3-FL, LST a, LST b, LST c, LST d, 3′SLNB, 6′SLNB, sialyl Lewis a, 3′SLacNAc, 6′SLacNAc and sialyl Lewis x. Even more preferably, the first sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably, the first sialylated MMO is selected from the group consisting of 3′SL, 6′SL, 3′SLNB, 6′SLNB, 3′SLacNAc and 6′SLacNAc. Even more preferably, the first sialylated MMO is 3′-sialyllactose (3′SL) or 6′-sialyllactose (6′SL), most preferably, the first sialylated MMO is 3′SL.

In an additional and/or more preferred alternative embodiment, the second sialylated MMO according to the disclosure is selected from a group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), monofucosylmonosialyllacto-N-octaose (sialyl Lewis a), 3′-sialyllactosamine (3′SLacNAc), 6′-sialyllactosamine (6′SLacNAc) and sialyl Lewis x. Even more preferably, the second sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably, the second sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably, the second sialylated MMO is selected from the group consisting of 3′SL, 6′SL, 3′SLNB, 6′SLNB, 3′SLacNAc and 6′SLacNAc. Even more preferably, the second sialylated MMO is 6′-sialyllactose (6′SL) or 3′-sialyllactose (3′SL), most preferably, the second sialylated MMO is 6′SL.

The method according to the disclosure does not exclude the administration of one or more additional MMOs, albeit it is not a prerequisite to achieve a reduction of the feed conversion ratio of a farm animal. In this regard, it is preferred that the method according to the disclosure comprises the administration of one or more sialylated, preferably two or more sialylated, more preferably two sialylated, MMOs as described herein and a fucosylated MMO as depicted herein. In an alternative embodiment, the sialylated MMO(s) is/are the sole MMO(s) administered to the farm animal according to the method of the disclosure.

Throughout the application and claims, unless specifically stated otherwise, the expression “a sialylated MMO” refers to one or more sialylated MMOs, preferably to two or more sialylated MMOs (i.e., a first sialylated MMO and a second sialylated MMO, optionally further sialylated MMOs), more preferably to two sialylated MMOs (i.e., a first sialylated MMO and a second sialylated MMO and no further sialylated MMOs).

In a more preferred embodiment of the disclosure, the method according to the disclosure comprises the step of administering to the farm animal a fucosylated mammalian milk oligosaccharide (MMO) and a sialylated mammalian milk oligosaccharide (MMO), preferably a fucosylated MMO and two or more sialylated MMOs (i.e., a first sialylated MMO and a second sialylated MMO, optionally further sialylated MMOs are administered), more preferably a fucosylated MMO and two sialylated MMOs (i.e., a first sialylated MMO and a second sialylated MMO). For the sake of clarity, the expression “more preferably two sialylated MMOs” in this context of the disclosure means that no additional sialylated MMOs are administered to the subject. It is preferred that the administered MMOs are administered as a mixture, more preferably as part of a composition, even more preferably as part of a composition according to the section titled “Composition.” It was surprisingly found in the disclosure, that administering a fucosylated MMO, in particular, 2′FL, and a sialylated MMO, in particular, the combination of 3′-sialyllactose (3′SL) and 6′-sialyllactose (6′SL), to a farm animal results in an even stronger decrease of the feed conversion ratio of the animal compared to the administration of solely the fucosylated MMO and compared to the administration of solely the sialylated MMO.

In this context of the disclosure, it is a preferred embodiment that the fucosylated MMO is as described earlier herein. In this context of the disclosure, it is an additional and/or preferred alternative embodiment that the sialylated MMO is as described earlier herein.

In a more preferred embodiment, the method according to the disclosure comprises administering a fucosylated MMO, a first sialylated MMO and a second sialylated MMO (preferably no further sialylated MMOs are administered). Preferably, the fucosylated MMO is as described earlier herein. Preferably, the first sialylated MMO is as the sialylated MMO as described earlier herein. Preferably, the second sialylated MMO is as the sialylated MMO as described earlier herein.

More preferably, the first sialylated MMO is selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x. Even more preferably, the first sialylated MMO is selected from the group consisting of 3′SL, 6′SL, 3′S-2′FL, 6′S-2′FL, 3′S-3-FL, 6′S-3-FL, LST a, LST b, LST c, LST d, 3′SLNB, 6′SLNB, sialyl Lewis a, 3′SLacNAc, 6′SLacNAc and sialyl Lewis x. Even more preferably, the first sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably, the first sialylated MMO is selected from the group consisting of 3′SL, 6′SL, 3′SLNB, 6′SLNB, 3′SLacNAc and 6′SLacNAc. Even more preferably, the first sialylated MMO is 3′-sialyllactose (3′SL) or 6′-sialyllactose (6′SL), most preferably, the first sialylated MMO is 3′SL.

More preferably, the second sialylated MMO is selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x. Even more preferably, the second sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably, the second sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably, the second sialylated MMO is selected from the group consisting of 3′SL, 6′SL, 3′SLNB, 6′SLNB, 3′SLacNAc and 6′SLacNAc. Even more preferably, the second sialylated MMO is 6′-sialyllactose (6′SL) or 3′-sialyllactose (3′SL), most preferably, the second sialylated MMO is 6′SL.

It is particularly preferred that the first sialylated MMO comprises a sialic acid, preferably a Neu5Ac, that is linked to a monosaccharide in an α-2,3-linkage, wherein the monosaccharide is preferably selected from galactose, N-acetylglucosamine and Neu5Ac, more preferably the monosaccharide is galactose or N-acetylglucosamine. Hence, the first sialylated MMO is preferably selected from the group consisting of 3′-sialyllactose (3′SL), 3′-sialyl-2′-fucosyllactose (3′S-2′FL), 3′-sialyl-3′-fucosyllactose (3′S-3′FL), LST a, LST d, 3′-sialyl-N-acetyllactosamine (3′SLacNAc), 3′-sialyl-3-fucosyllactose and 3′-sialyllacto-N-biose (3′SLNB), more preferably selected from the group consisting of 3′-sialyllactose (3′SL), LST a, LST d, 3′-sialyl-N-acetyllactosamine (3′SLacNAc) and 3′-sialyllacto-N-biose (3′SLNB), even more preferably selected from the group consisting of 3′-sialyllactose (3′SL), 3′-sialyl-N-acetyllactosamine (3′SLacNAc) and 3′-sialyllacto-N-biose (3′SLNB), most preferably wherein the first sialylated MMO is 3′-sialyllactose (3′SL).

It is particularly preferred that the second sialylated MMO comprises a sialic acid, preferably a Neu5Ac, that is linked to a monosaccharide in an α-2,6-linkage, wherein the monosaccharide is preferably selected from galactose, N-acetylglucosamine and Neu5Ac, more preferably the monosaccharide is galactose or N-acetylglucosamine. Hence, the second sialylated MMO is preferably selected from the group consisting of 6′-sialyllactose (6′SL), 6′-sialyl-2′-fucosyllactose (6′S-2′FL), 6′-sialyl-3′-fucosyllactose (6′S-3′FL), Neu5Ac-α2,6-[GlcNAc-b1,3]Gal-b1,4-Glc, LST b, LST c, 6′-sialyl-N-acetyllactosamine (6′SLacNAc) and 6′-sialyllacto-N-biose (6′SLNB), more preferably selected from the group consisting of 6′-sialyllactose (6′SL), LST b, LST c, 6′-sialyl-N-acetyllactosamine (6′SLacNAc) and 6′-sialyllacto-N-biose (6′SLNB), even more preferably selected from the group consisting of 6′-sialyllactose (6′SL), 6′-sialyl-N-acetyllactosamine (6′SLacNAc) and 6′-sialyllacto-N-biose (6′SLNB), most preferably wherein the second sialylated MMO is 6′-sialyllactose (6′SL).

The method according to the disclosure does not exclude the administration of one or more additional MMOs, albeit it is not a prerequisite to achieve a high reduction of the feed conversion ratio of a farm animal. In this regard, it is preferred that the method according to the disclosure comprises the administration of a fucosylated MMO and a sialylated MMO as described herein. It is more preferred that the method according to the disclosure comprises the administration of a fucosylated MMO, a first sialylated MMO and a second sialylated MMO (preferably, no additional sialylated MMOs are administered) as described herein. In a most preferred embodiment, the method according to the disclosure, the fucosylated MMO, the first sialylated MMO and the second sialylated MMO are the sole MMOs administered to the farm animal according to the method of the disclosure.

The aforementioned fucosylated MMOs are commercially available and/or the production/purification of these oligosaccharides has been described and hence allows the skilled person to produce/obtain any of the fucosylated oligosaccharides accordingly. For example (each reference is incorporated by reference):
2′FL: Zhou et al., 2021, ACS Synth. Biol. 10(3): p. 447-458; Lu et al., 2021, ACS Synth. Biol. 10(5): p. 923-938; WO2022/034073; Carbosynth (OF06739)
3-FL: Zhou et al., 2021; ACS Synth. Biol. 10(3): p. 447-458, Lu et al., 2021, ACS Synth. Biol. 10(5): p. 923-938; WO2022/034073; Carbosynth (OF05673)
diFL: WO2022/034073
2′FlacNAc: WO 2022/034075
3FlacNAc: WO 2022/034075
diFLacNAc: WO 2022/034075
2′FLNB: WO 2022/034075
4FLNB: WO 2022/034075
diFLNB: WO 2022/034075
LNFP I: Derya et al., 2020, J. Biotech. 319: p. 31-38; Carbosynth (OL05676)
GalNAc-LNFP I: WO 2022/034077
Gal-LNFP I: WO 2022/034077
LNFP II: Zeuner et al., 2018, ACS Synth. Biol. 10(3): p. 447-458
LNFP III: Zeuner et al., 2018, ACS Synth. Biol. 10(3): p. 447-458; Carbosynth (OL04212)
LNFP V: WO2020/115671; Carbosynth (OL06817)
LNDFH I: Huang et al., 2021, ACS Catal. 11(5): p. 2631-2643; Carbosynth (OL01664)
LNDFH II: Yu et al., 2017, Chem. Comm. 53(80): p. 11012-11015; Huang et al., 2021, ACS Catal. 11(5): p. 2631-2643; Carbosynth (OL06826)
MFLNH III: Carbosynth (OM05898)
DFLNH (a): Carbosynth (OD05375)
DFLNH: Carbosynth (OD06532)
TFLNH: Isosep (57/18-0010)
LNnFP I: Elicityl (GLY033-2-90%)
LNnFP V: Dumon et al., 2001, Glyconj. J. 18(6): p. 465-474
LNnDFH: Dumon et al., 2001, Glyconj. J. 18(6): p. 465-474

The aforementioned sialylated MMOs are commercially available and/or the production/purification of these oligosaccharides has been described and hence allows the skilled person to produce/obtain any of the sialylated oligosaccharides accordingly. For example (each reference is incorporated by reference):
3′SL: Zhang et al., 2022, ACS Synth. Biol. 11(8): p. 2837-2845; Carbosynth (OS04397)
6′SL: Guo et al., 2018, Appl. Environ. Microbiol. 84(13): e00071-18; Carbosynth (OS04398)
3,6-disialyllactose: Pan et al., 2006, Carbohydr. Res. 341(6): p. 730-737
6,6′-disialyllactose: Drouillard et al., 2010, Carbohydr. Res. 345(10): p. 1394-1399
8,3-disialyllactose: Carbosynth (OD45739)
3′S-3-FL: Biosynth/Cymit Quimica (3D-OSO1065)
LST a: Carbosynth (OL03882)
LST b: Carbosynth (OL03877)
LST c: WO2016/199071; Carbosynth (OL06570)
LST d: Carbosynth (OS158776)
3′SLNB: Carbosynth (OS35289)
Sialyl lewis a: Carbosynth (OS00745)
Sialyl lewis x: Yu et al., 2017, Chem. Commun. (Camb) 53(80): p. 11012-11015; Carbosynth (OS04058)

In an additional and/or more preferred alternative embodiment, the fucosylated MMO and/or the sialylated MMO, preferably the fucosylated MMO and the sialylated MMO, according to the disclosure has/have been isolated from a microbial cultivation or fermentation, cell culture, enzymatic reaction or chemical reaction. Alternatively, the fucosylated MMO and/or the sialylated MMO, preferably the fucosylated MMO and the sialylated MMO, of the disclosure has/have been isolated by, e.g., chromatography or filtration technology from a natural source such as a human or animal milk, preferably animal milk. Isolation of the fucosylated MMO and/or the sialylated MMO, preferably the fucosylated MMO and the sialylated MMO, typically results in a solution containing the oligosaccharide(s). Such a solution can, for example, be obtained by a method comprising the steps of:
(a) cultivating at least one cell as defined herein, preferably a single cell, that is capable to produce the fucosylated MMO, and/or the sialylated MMO, preferably the fucosylated MMO and the sialylated MMO, in a suitable cultivation medium to form a cultivation broth, preferably wherein the cell is metabolically engineered for the production of the fucosylated MMO and/or the sialylated MMO, preferably the fucosylated MMO and the sialylated MMO, and
(b) purifying the fucosylated MMO and/or the sialylated MMO, preferably the fucosylated MMO and the sialylated MMO, from the cultivation broth by:
   (i) clarifying the cultivation broth, and
   (ii) removing salts and/or medium components form the clarified cultivation broth, and/or
(c) concentrating the MMO(s) in the clarified cultivation broth,
thereby providing a solution comprising a purified fucosylated oligosaccharide and/or a purified sialylated MMO, preferably a purified fucosylated MMO and a purified sialylated MMO.

In the context of the disclosure, “clarifying the cultivation broth” refers to the removal of suspended particulates and contaminants, particularly cells, cell components, insoluble metabolites and debris produced by culturing the cell according to the disclosure. Clarification is preferably one or more of centrifugation, flocculation, decantation and/or filtration.

In the context of the disclosure, “removing salts and/or medium components form the clarified cultivation broth” refers to removing substantially all the proteins, as well as peptides, amino acids, RNA and DNA and any endotoxins and glycolipids from the clarified cultivation broth, preferably after it has been. In this step, proteins and related impurities can be removed from the saccharide in a conventional manner. Preferably, proteins, salts, by-products, color, endotoxins and other related impurities are removed from the oligosaccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g., using slab-polyacrylamide or sodium dodecyl sulphate-polyacrylamide gel electrophoresis (PAGE)), affinity chromatography (using affinity ligands including, e.g., DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, the oligosaccharide remains in the oligosaccharide containing clarified cultivation broth.

Further purification of the oligosaccharide may be accomplished, for example, by use of (activated) charcoal or carbon, nanofiltration, ultrafiltration, electrophoresis, enzymatic treatment or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the product.

Several drying techniques are known to the skilled person that can be used to obtain powder from a solution containing the oligosaccharide(s). Powder is preferably obtained by spray drying, freeze drying, spray freeze-drying, crystallization, lyophilization, band or belt drying, drum or roller drying, and/or agitated thin film drying, preferably by spray drying, drum or roller drying, or agitated thin film drying, more preferably by spray drying or agitated thin film drying, most preferably by spray drying, of a solution containing the oligosaccharide(s). Preferably, the oligosaccharide(s) according to the disclosure constitutes ≥70.0%, preferably ≥75.0%, more preferably ≥80.0%, (w/w) of the powder. Preferably, the powder contains ≤15 wt. %, preferably ≤10 wt. %, more preferably ≤9 wt. %, more preferably ≤8 wt. %, more preferably ≤7 wt. %, even more preferably ≤5 wt. %, even more preferably ≤4 wt. % of liquid, even more preferably ≤3 wt. % of liquid, even more preferably 2 wt. % of liquid, most preferably ≤1 wt. %, preferably wherein the liquid is water.

In an additional and/or more preferred alternative embodiment, the fucosylated MMO according to the disclosure has been produced, preferably in vitro and/or ex vivo, by a cell, preferably a single cell, wherein the cell is preferably chosen from the group consisting of a microorganism, a plant cell, an animal cell and a protozoan cell. In other words, the fucosylated oligosaccharide of the disclosure has been produced by an in vitro and/or ex vivo culture of cells, wherein the cells are preferably chosen from the group consisting of a microorganism, a plant cell, an animal cell or a protozoan cell.

In an additional and/or more preferred alternative embodiment, the sialylated MMO according to the disclosure has been produced, preferably in vitro and/or ex vivo, by a cell, preferably a single cell, wherein the cell is preferably chosen from the group consisting of a microorganism, a plant cell, an animal cell and a protozoan cell. In other words, the sialylated oligosaccharide of the disclosure has been produced by an in vitro and/or ex vivo culture of cells, wherein the cells are preferably chosen from the group consisting of a microorganism, a plant cell, an animal cell or a protozoan cell.

Preferably, the cell is a microorganism. Preferably, the microorganism is selected from a group consisting of a bacterium, a yeast and a fungus. Further, it is preferred that the cell is genetically engineered for the production of the fucosylated MMO and/or sialylated MMO according to the disclosure. Throughout the description and claims, the terms “has been isolated” and “is obtained” are interchangeably used, unless specifically stated otherwise.

The latter bacterium preferably belongs to the phylum of the Proteobacteria or the phylum of the Firmicutes or the phylum of the Cyanobacteria or the phylum Deinococcus-Thermus, preferably belongs to the phylum of the Proteobacteria. The latter bacterium belonging to the phylum Proteobacteria preferably belongs to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild-type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably the disclosure specifically relates to a mutated and/or transformed Escherichia coli strain as indicated above wherein the E. coli strain is a K12 strain. More specifically, the disclosure relates to a mutated and/or transformed Escherichia coli strain as indicated above wherein the K12 strain is E. coli MG1655. The latter bacterium belonging to the phylum Firmicutes preferably belongs to the Bacilli, preferably from the species Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast preferably belongs to the genus Saccharomyces (with members like, e.g., Saccharomyces cerevisiae, S. bayanus, S. boulardii), Pichia (with members like, e.g., Pichia pastoris, P. anomala, P. kluyveri), Komagataella, Hansunella, Kluyveromyces (with members like, e.g., Kluyveromyces lactis, K. marxianus, K. thermotolerans), Yarrowia (like, e.g., Yarrowia lipolytica), Eremothecium, Zygosaccharomyces, Starmerella (like, e.g., Starmerella bombicola) or Debaromyces. The latter yeast is preferably selected from Pichiapastoris, Yarrowia lipolitica, Saccharomyces cerevisiae and Kluyveromyces lactis. The latter fungus preferably belongs to the genus Rhizopus, Dictyostelium, Penicillium, Mucor or Aspergillus. “Plant cells” includes cells of flowering and non-flowering plants, as well as algal cells, for example, Chlamydomonas, Chlorella, etc. Preferably, the plant cell is a tobacco, alfalfa, rice, cotton, rapeseed, tomato, corn, maize or soybean cell. The latter animal cell is preferably derived from non-human mammals (e.g., cattle, buffalo, pig, sheep, mouse, rat), birds (e.g., chicken, duck, ostrich, turkey, pheasant), fish (e.g., swordfish, salmon, tuna, sea bass, trout, catfish), invertebrates (e.g., lobster, crab, shrimp, clams, oyster, mussel, sea urchin), reptiles (e.g., snake, alligator, turtle), amphibians (e.g., frogs) or insects (e.g., fly, nematode) or is a genetically modified cell line derived from human cells excluding embryonic stem cells. Both human and non-human mammalian cells are preferably chosen from the group comprising an epithelial cell like, e.g., a mammary epithelial cell, mammary myoepithelial cell, mammary progenitor cell, an embryonic kidney cell (e.g., HEK293 or HEK 293T cell), a fibroblast cell, a COS cell, a Chinese hamster ovary (CHO) cell, a murine myeloma cell like, e.g., an N20, SP2/0 or YB2/0 cell, an NIH-3T3 cell, a non-mammary adult stem cell or derivatives thereof such as described in WO21067641, preferably mesenchymal stem cell or derivates thereof as described in WO21067641. The insect cell is preferably derived from Spodoptera frugiperda like, e.g., Sf9 or Sf21 cells, Bombyx mori, Mamestra brassicae, Trichoplusia ni like, e.g., BTI-TN-5B1-4 cells or Drosophila melanogaster like, e.g., Drosophila S2 cells. The latter protozoan cell preferably is a Leishmania tarentolae cell.

In an additional and/or more preferred alternative embodiment, the fucosylated MMO of the disclosure has been produced, preferably in vitro and/or ex vivo, by a mammary epithelial cell, mammary myoepithelial cell and/or mammary progenitor cell, preferably wherein the cell is generated from non-mammary adult stem cells, more preferably wherein the cell is generated from mesenchymal stem cells.

In an additional and/or more preferred alternative embodiment, the sialylated MMO of the disclosure has been produced, preferably in vitro and/or ex vivo, by a mammary epithelial cell, mammary myoepithelial cell and/or mammary progenitor cell, preferably wherein the cell is generated from non-mammary adult stem cells, more preferably wherein the cell is generated from mesenchymal stem cells.

Such cells are well-known to the skilled person, and it is, in this regard, referred to, for example, in WO2021/067641 and WO2021/242866 (mammary epithelial cells derived from non-mammary adult stem cells, preferably from mesenchymal stem cells) and WO2021/142241 (mammary epithelial cells, mammary myoepithelial cells, and mammary progenitor cells).

In an even more preferred embodiment, the fucosylated MMO of the disclosure has been produced, preferably in vitro and/or ex vivo, by a microorganism cell, preferably the microorganism is a bacterium, a yeast or a fungus, more preferably the microorganism is a bacterium or a yeast, even more preferably, the microorganism is a bacterium, most preferably, the microorganism is Escherichia coli.

In an even more preferred embodiment, the sialylated MMO of the disclosure has been produced, preferably in vitro and/or ex vivo, by a microorganism cell, preferably, the microorganism is a bacterium, a yeast or a fungus, more preferably, the microorganism is a bacterium or a yeast, even more preferably, the microorganism is a bacterium, most preferably, the microorganism is Escherichia coli.

In the context of the disclosure, it is also within the scope of the disclosure that two or more different cells (preferably as defined herein), produce the fucosylated MMO and/or sialylated MMO according to the disclosure. It is, however, preferred that a single cell produces the oligosaccharide(s), i.e., a single culture of the cell produces the oligosaccharide(s) of the disclosure.

In a preferred embodiment of the disclosure, the fucosylated MMO according to the disclosure is linked, preferably chemically linked, to a carrier for delivery of the MMO. In an additional and/or preferred alternative embodiment of the disclosure, the sialylated MMO according to the disclosure is linked, preferably chemically linked, to a carrier for delivery of the MMO. In an additional and/or preferred alternative embodiment of the disclosure, the first sialylated MMO according to the disclosure is linked, preferably chemically linked, to a carrier for delivery of the MMO. In an additional and/or preferred alternative embodiment of the disclosure, the second sialylated MMO according to the disclosure is linked, preferably chemically linked, to a carrier for delivery of the MMO.

Preferably, the carrier is a ceramide-based carrier or a polypeptide-based carrier, more preferably the carrier is a ceramide-based carrier. Preferably, the polypeptide-based carrier is epsilon-polylysine, alfa-polylysine, poly(aspartic acid), polyglutamic acid or polyornithine. These carriers are commercially available (e.g., Sigma-Aldrich, Carbosynth). The ceramide-based carrier is preferably selected from a group consisting of d18:1/16:0, t18:0-16:0, t18:0-h16:0, t18:0-h22:0 and t18:0-h24:0. These ceramide carriers are commercially available and well-known to the skilled person and are, for example, described in WO2010/037785, which is incorporated by reference. d18:1/16:0 is also known as C16 ceramide and N-palmitoylsphingosine and, therefore, interchangeably used herein. t18:0-16:0 is also known as C16 phytoceramide and N-hexadecanoyl phytosphingosine and, therefore, interchangeably used herein. t18:0-h16:0, t18:0-h22:0 and t18:0-h24:0 are glycosylinositolphosphoceramides (GIPCs). As known to the skilled person, “d” and “t” refer to the hydroxylation state of the whole ceramide or long-chain base moiety (d is 2 groups, t is 3 groups), whereas “h” denotes a hydroxylation of the fatty acyl group.

The fucosylated MMO and optionally any, preferably all, further MMOs, is preferably encapsulated (i.e., forming an encapsulate). This is particularly advantageous to protect the oligosaccharide(s) from conditions relating to the manufacturing or storing of a composition as described in the section titled “Composition,” especially when the composition is a nutritional composition (it is referred to in the section titled “Composition”). Such conditions typically entail a high pressure (e.g., a pressure of above 10 bar during extrusion), a high temperature (e.g., a temperature above 100° C. during extrusion and baking), a pH lower than 5, a pH higher than 8, and/or a high moisture content (e.g., wet extrusion). Moreover, other molecules within a composition, especially a nutritional composition, such as proteins could chemically interact with the fucosylated MMO (and any other further MMO if present) especially under the harsh conditions during manufacturing of a nutritional composition. Hence, throughout the application and claims, the “fucosylated MMO” is preferably replaced with “encapsulated fucosylated MMO.” If any further MMO is present, then it is preferred that all MMOs present in the composition are encapsulated, preferably all MMOs are co-encapsulated.

Likewise, the sialylated MMO or the sialylated MMOs and optionally any, preferably all, further MMOs, is preferably encapsulated (i.e., forming an encapsulate).

The encapsulated is particularly advantageous when the composition according to the disclosure is a nutritional composition (it is referred to in the section titled “Composition”), which is manufactured by extrusion (dry or wet, preferably wet).

It is preferred that the encapsulate is of the core-shell type, i.e., carrier material forms a shell around the active agent (i.e., the MMO(s) according to the disclosure), preferably wherein the core has a median diameter (D(v,0.5)) of 125-250 μm, preferably 150-250 μm, more preferably 165-250 μm, most preferably 165-225 μm, and wherein the encapsulate has a median diameter (D(v,0.5)) that is 20-100 μm, preferably 20-75 μm, more preferably 35-75 μm, longer than the median diameter of the core.

The carrier material preferably comprises a hot melt material, more preferably an oil and/or a wax, even more preferably a hydrogenated oil and/or a wax, optionally further comprising starch, preferably wherein the starch constitutes less than 15% (w/w) of the carrier material, more preferably the starch constitutes less than 10% (w/w) of the carrier material.

The oil is preferably selected from the group consisting of palm oil, sunflower oil, soybean oil, rapeseed oil, coconut oil, babassu oil, palm kernel oil, maize oil, sesame oil and cottonseed oil; more preferably, the oil is selected from the group consisting of palm oil, sunflower oil, soybean oil and rapeseed oil; even more preferably, the oil is palm oil, most preferably, the oil is hydrogenated palm oil.

The wax is preferably selected from the group consisting of Candelilla wax, Carnauba wax, beeswax, rice bran wax, paraffin wax, jojoba wax, microcrystalline wax and japan wax; more preferably, the wax is Candelilla wax or Carnauba wax; most preferably, the wax is Candelilla wax.

It is further preferred that the fucosylated MMO or all MMOs present in the encapsulate constitute 5-50% (w/w), preferably 10-40% (w/w), more preferably 20-40% (w/w), even more preferably 20-35% (w/w), of the total weight of the encapsulate.

### Method for Reducing FCR: General

In an embodiment of the first aspect of the disclosure, the method reduces the feed conversion ratio (FCR) of a farm animal, wherein the method comprises the step of administering to the farm animal: (i) a fucosylated MMO, (ii) a sialylated MMO, or (iii) a fucosylated MMO and a sialylated MMO as described herein (it is referred to in the section titled “Oligosaccharide”).

When a fucosylated MMO and a sialylated MMO according to the disclosure (it is referred to in the section titled “Oligosaccharide”) are administered to the farm animal, the fucosylated MMO and the sialylated MMO are preferably administered on the same day, more preferably administered simultaneously.

Simultaneous administration can be achieved by providing the fucosylated MMO and the sialylated MMO in the same composition (the composition is preferably as described in the section titled “Composition”).

Further, it is a preferred embodiment that the amount of the sialylated MMO administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal.

Further, it is a more preferred embodiment that the amount of all sialylated MMOs administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal.

Further, it is an even more preferred embodiment that the amount of the fucosylated MMO administered to the animal is lower than the amount of the sialylated MMO, preferably the amount of the fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of the sialylated MMO.

Further, it is an even more preferred embodiment that the amount of the fucosylated MMO administered to the animal is lower than the amount of all sialylated MMOs administered to the animal, preferably the amount of the fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of all sialylated MMOs administered to the animal.

When a first sialylated MMO and a second sialylated MMO according to the disclosure (it is referred to in the section titled “Oligosaccharide”) are administered to the farm animal, the first sialylated MMO and the second sialylated MMO are preferably administered on the same day, more preferably administered simultaneously.

Further, it is a preferred embodiment that the ratio between the amount of the first sialylated MMO administered to the animal and the amount of the second sialylated MMO administered to the animal is similar to this ratio as found in the mother's milk of the same species. Throughout the application and claims, unless specifically stated otherwise, a “similar” ratio preferably refers to a ratio that is identical or falls in the range of ratio +/−20% of the ratio, preferably ratio +/−10% of the ratio. When the animal is a bovine, the ratio is preferably 1.1 to 6.0, more preferably 1.1 to 5.0, even more preferably 1.25 to 4.0, even more preferably 1.25 to 3.0, even more preferably 1.5 to 3.0, most preferably 1.5 to 2.5. When the animal is a horse, the ratio is preferably 1.1 to 15.0, more preferably 1.1 to 12.5, even more preferably 1.1 to 10.0, even more preferably 1.5 to 10.0, even more preferably 2.5 to 10, even more preferably 5.0 to 10.0, even more preferably 7.5 to 10.0, most preferably 8.0 to 10.0. When the animal is a pig, the ratio is preferably 7.5-40.0, more preferably 7.5-35.0, even more preferably 10.0-35.0, even more preferably 15.0-35.0, even more preferably 20.0-35.0, most preferably 25.0-35.0. When the animal is a goat, the ratio is preferably 0.25-5.0, more preferably 0.25-3.0, even more preferably 0.25-2.0, even more preferably 0.25-1.5, most preferably 0.5-1.5.

Further, it is a more preferred embodiment that the amount of the first sialylated MMO administered to the animal is higher than the amount of the second sialylated MMO, preferably the amount of the first sialylated MMO is 1.1 to 40.0, more preferably 1.1 to 35.0, most preferably 1.5 to 35.0, times the amount of the second sialylated MMO administered to the animal.

Further, it is an even more preferred embodiment that the amount of the first sialylated MMO administered to the animal is higher than the amount of the second sialylated MMO, preferably the amount of the first sialylated MMO is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of the second sialylated MMO.

When a fucosylated MMO and a first sialylated MMO according to the disclosure (it is referred to in the section titled “Oligosaccharide”) are administered to the farm animal, the fucosylated MMO and the first sialylated MMO are preferably administered on the same day, more preferably administered simultaneously.

Further, it is a preferred embodiment that the amount of the first sialylated MMO administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal.

Further, it is a more preferred embodiment that the amount of the fucosylated MMO administered to the animal is lower than the amount of the first sialylated MMO, preferably the amount of the fucosylated MMO is 1.1 to 10.0, preferably 1.5-7.5, more preferably 2.5-7.5, most preferably 4.0-7.0, times lower than the amount of the first sialylated MMO.

When a fucosylated MMO, a first sialylated MMO and a second sialylated MMO according to the disclosure (it is referred to in the section titled “Oligosaccharide”) are administered to the farm animal, the fucosylated MMO, the first sialylated MMO and the second sialylated MMO are preferably administered on the same day, more preferably administered simultaneously.

Further, it is a preferred embodiment that the ratio between the amount of the first sialylated MMO administered to the animal and the amount of the second sialylated MMO administered to the animal is similar to this ratio as found in the mother's milk of the same species as described earlier herein. More preferably, the amount of the first sialylated MMO administered to the animal is higher than the amount of the second sialylated MMO, preferably the amount of the first sialylated MMO is 1.1 to 40.0, more preferably 1.1 to 35.0, most preferably 1.5 to 35.0, times higher than the amount of the second sialylated MMO. Even more preferably, the amount of the first sialylated MMO administered to the animal is higher than the amount of the second sialylated MMO, preferably the amount of the first sialylated MMO is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of the second sialylated MMO.

It is an additional and/or preferred alternative embodiment that the amount of the first sialylated MMO administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal. Preferably, the amount of the fucosylated MMO administered to the animal is lower than the amount of the first sialylated MMO, preferably the amount of the fucosylated MMO is 1.1 to 10.0, preferably 1.5-7.5, more preferably 2.5-7.5, most preferably 4.0-7.0, times lower than the amount of the first sialylated MMO.

It is an additional and/or preferred alternative embodiment that the amount of the second sialylated MMO administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal. Preferably, the amount of the fucosylated MMO administered to the animal is lower than the amount of the second sialylated MMO, preferably the amount of the fucosylated MMO is 1.1 to 10.0, preferably 1.5-7.5, more preferably 1.5-5.0, most preferably 2.5-5.0, times lower than the amount of the second sialylated MMO.

It is a more preferred embodiment that the total amount of both first sialylated MMO and second sialylated MMO is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal. Preferably, the amount of the fucosylated MMO administered to the animal is lower than the total amount of both first sialylated MMO and second sialylated MMO, preferably the amount of the fucosylated MMO is 1.1 to 15.0, preferably 1.5 to 15.0, more preferably 2.5 to 15.0, even more preferably 5.0 to 15.0, even more preferably 5.0 to 12.5, most preferably 5.0 to 10.0, times lower than the total amount of both first sialylated MMO and second sialylated MMO.

It is an even more preferred embodiment that the amount of all sialylated MMOs that are administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated administered to the animal. Preferably, the amount of the fucosylated MMO administered to the animal is lower, preferably 1.1 to 15.0 times lower, more preferably 1.5 to 15.0 times lower, even more preferably 2.5 to 15.0 times lower, even more preferably 5.0 to 15.0 times lower, even more preferably 5.0 to 12.5 times lower, most preferably 5.0 to 10.0 times lower, than the amount of all sialylated MMOs administered to the animal.

### Method to Reduce FCR: Fucosylated MMO

In a preferred embodiment, the method according to the disclosure comprises administering an effective amount of the fucosylated MMO (it is referred to in the section titled “Oligosaccharide”) according to the disclosure to the farm animal (it is referred to in the section titled “Subject”). As understood by the skilled person, an “effective amount” as used throughout the application and claims is the amount of the oligosaccharide that is required to confer a reduction in the FCR of the farm animal as described in the present application. Effective amounts vary, as recognized by those skilled in the art, depending on the subject among other known factors.

In an additional and/or preferred alternative embodiment, the fucosylated MMO is administered to the animal at least once, preferably at least twice, between birth (0 weeks) and 5 weeks of age, preferably between birth and 4 weeks of age, more preferably between birth and 3 weeks of age.

In a more preferred embodiment, the fucosylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g. More preferably, the fucosylated MMO is administered at a daily dose of 0.001-15.0 g, preferably 0.001-10.0 g, more preferably 0.001-5.0 g, even more preferably 0.001-2.0 g, even more preferably 0.001-1.0 g, even more preferably 0.005-1.0 g, most preferably 0.010-1.0 g. Even more preferably, the fucosylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.

In the context of the disclosure, an amount of an oligosaccharide according to the disclosure expressed in a number of grams or milligrams per daily dose as used herein means that the amount of the oligosaccharide is such that when administering the daily dosage to a subject, the subject will be administered with the number of grams or milligrams of the oligosaccharide. In other words, if the daily dosage is, for example, 50 mg, then the subject receives in total 50 mg per day. This may be in one or more portions. So, if the daily dosage is 50 mg divided over 2 portions, then a single serving consists of 25 mg, a daily serving consists of 2 of such single servings.

In an additional and/or more preferred alternative embodiment, the fucosylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of the animal.

In an even more preferred embodiment, the fucosylated MMO is administered at a daily dose of 0.01-1.0 g, preferably 0.01-0.75 g, more preferably 0.01-0.65 g, even more preferably 0.01-0.60 g, even more preferably 0.01-0.50 g, even more preferably 0.015-0.50 g, even more preferably 0.015-0.40 g, even more preferably 0.015-0.30 g, even more preferably 0.015-0.20 g, most preferably 0.015-0.10 g. This embodiment is, for example, particularly preferred if the animal is a bovine or a horse, more preferably wherein the animal is a calf or a foal, most preferably wherein the animal is a calf.

In an additional and/or even more preferred alternative embodiment, the fucosylated MMO is administered at a daily dose of 0.1-50.0 mg, preferably 0.1-30.0 mg, more preferably 0.1-25.0 mg, even more preferably 0.1-20.0 mg, even more preferably 0.1-15.0 mg, even more preferably 0.1-10.0 mg, even more preferably 0.25-10.0 mg, most preferably 0.5-10.0 mg, per kg bodyweight of the animal. This embodiment is, for example, particularly preferred if the animal is a bovine or a horse, more preferably wherein the animal is a calf or a foal, most preferably wherein the animal is a calf.

In an even more preferred alternative embodiment, the fucosylated MMO is administered at a daily dose of 0.01-5.0 g, preferably 0.1-2.5 g, more preferably 0.1-1.5 g, even more preferably 0.1-1.2 g, even more preferably 0.2-1.2 g, even more preferably 0.3-1.2 g, even more preferably 0.4-1.2 g, most preferably 0.5-1.2 g. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In an additional and/or even more preferred alternative embodiment, the fucosylated MMO is administered at a daily dose of 0.1-150.0 mg, preferably 0.1-125.0 mg, more preferably 1.0-125.0 mg, even more preferably 5.0-125.0 mg, even more preferably 5.0-100.0 mg, even more preferably 5.0-80.0 mg, most preferably 10.0-80.0 mg, per kg bodyweight of the animal. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In an even more preferred alternative embodiment, the fucosylated MMO is administered at a daily dose of 0.0001-1.0 g, preferably 0.0001-0.50 g, more preferably 0.0001-0.10 g, even more preferably 0.0001-0.075 g, even more preferably 0.0001-0.050 g, even more preferably 0.0002-0.050 g, even more preferably 0.0005-0.050 g, most preferably 0.0005-0.025 g. This embodiment is, for example, particularly preferred if the animal is a fish or a crustacean, more preferably a fish, even more preferably a fry or a fingerling, even more preferably a fingerling.

In an additional and/or even more preferred alternative embodiment, the fucosylated MMO is administered at a daily dose of 0.1-70.0 mg, preferably 0.1-60.0 mg, more preferably 0.1-50.0 mg, even more preferably 0.5-50.0 mg, even more preferably 1.0-50.0 mg, even more preferably 2.5-50.0 mg, even more preferably 5.0-50.0 mg, even more preferably 10.0-50.0 mg, most preferably 10.0-40.0 mg, per kg bodyweight of the animal. This embodiment is, for example, particularly preferred if the animal is a fish or a crustacean, more preferably a fish, even more preferably a fry or a fingerling, even more preferably a fingerling.

In an even more preferred embodiment, the fucosylated MMO is administered to the animal for at least 1 week, preferably at least 2 weeks, more preferably at least 3 weeks, even more preferably at least 4 weeks, most preferably at least 6 weeks.

In an additional and/or even more preferred alternative embodiment, the sialylated MMO/first sialylated MMO/second sialylated MMO is administered to the animal for ≤32 weeks, preferably ≤28 weeks, more preferably ≤24 weeks, even more preferably ≤20 weeks, even more preferably ≤16 weeks, most preferably ≤12 weeks.

In an even more preferred embodiment, the fucosylated MMO is administered to the animal for 1-30, preferably 2-30, more preferably 3-30, even more preferably 4-30, even more preferably 5-30, even more preferably 6-30, even more preferably 6-24, even more preferably 6-18, most preferably 6-12, consecutive weeks.

In an even more preferred embodiment, the fucosylated MMO is administered to the farm animal for:
if subject is bovine: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
if subject is a pig: 1-18, preferably 1-16, more preferably 1-14, even more preferably 1-12, even more preferably 2-12, even more preferably 3-12, even more preferably 4-12, most preferably 6-12, consecutive weeks;
if subject is a sheep: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
if subject is a goat: 1-18, preferably 1-16, more preferably 1-14, even more preferably 1-12, even more preferably 2-12, even more preferably 3-12, even more preferably 4-12, most preferably 6-12, consecutive weeks;
if subject is a horse: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
if subject is a camelid: 1-14, preferably 1-12, more preferably 2-12, even more preferably 3-12, even more preferably 4-12, most preferably 6-12, consecutive weeks;
if subject is a rabbit: 1-12, preferably 2-12, more preferably 3-12, even more preferably 4-12, most preferably 6-12, consecutive weeks;
if subject is a fish or a crustacean: 1-36, preferably 1-32, more preferably 1-24, even more preferably 1-18, even more preferably 1-12, even more preferably 2-12, even more preferably 3-12, even more preferably 6-12, consecutive months;
if subject is poultry (but not a turkey): 1-8, preferably 1-7, more preferably 2-7, even more preferably 3-7, most preferably 4-7, consecutive weeks;
if subject is a turkey: 1-18, preferably 1-16, more preferably 1-14, even more preferably 1-12, even more preferably 2-12, even more preferably 3-12, even more preferably 4-12, most preferably 6-12, consecutive weeks.

Preferably, the fucosylated MMO according to the disclosure is administered at least once a week (i.e., at least one daily dosage is administered in a week), more preferably at least once every 3 days, even more preferably at least once every 2 days, most preferably at least once daily (i.e., a daily dosage is administered every day).

In an even more preferred embodiment, the fucosylated MMO is administered to the animal until the age of slaughtering.

### Method to Reduce FCR: Sialylated MMO

In a preferred embodiment, the method according to the disclosure comprises administering an effective amount of the sialylated MMO (it is referred to in the section titled “Oligosaccharide”) according to the disclosure to the farm animal (it is referred to in the section titled “Subject”).

In an additional and/or preferred alternative embodiment, the method according to the disclosure comprises administering an effective amount of the first sialylated MMO (it is referred to in the section titled “Oligosaccharide”) according to the disclosure to the farm animal (it is referred to in the section titled “Subject”).

In an additional and/or preferred alternative embodiment, the method according to the disclosure comprises administering an effective amount of the first sialylated MMO and an effective amount of the second sialylated MMO (it is referred to in the section titled “Oligosaccharide”) according to the disclosure to the farm animal (it is referred to in the section titled “Subject”).

The following embodiments described in the section titled “Method to reduce FCR: sialylated MMO” apply to “sialylated MMO,” “first sialylated MMO” and “second sialylated MMO” as described in the application and claims (i.e., according to the disclosure), unless specifically stated otherwise. For the sake of clarity, “sialylated MMO/first sialylated MMO/second sialylated MMO” (i.e., sialylated MMO or first sialylated MMO or second sialylated MMO) is indicated accordingly and each of the embodiments is hence considered to be explicitly disclosed in the context of “a sialylated MMO,” “a first sialylated MMO” and “a second sialylated MMO” separately and in combination with each other, unless specifically stated otherwise.

In an additional and/or preferred alternative embodiment, the sialylated MMO/first sialylated MMO/second sialylated MMO is administered to the animal at least once, preferably at least twice, between birth (0 weeks) and 5 weeks of age, preferably between birth and 4 weeks of age, more preferably between birth and 3 weeks of age.

In a more preferred embodiment, the sialylated MMO/first sialylated MMO/second sialylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g. More preferably, the sialylated MMO/first sialylated MMO/second sialylated MMO is administered at a daily dose of 0.001-15.0 g, preferably 0.001-10.0 g, more preferably 0.001-5.0 g, even more preferably 0.001-2.0 g, even more preferably 0.001-1.0 g, even more preferably 0.005-1.0 g, most preferably 0.010-1.0 g. Even more preferably, the sialylated MMO/first sialylated MMO/second sialylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.

In an additional and/or more preferred alternative embodiment, the sialylated MMO/first sialylated MMO/second sialylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of the animal.

In an even more preferred embodiment, the sialylated MMO/first sialylated MMO is administered at a daily dose of 0.01-1.0 g, preferably 0.01-0.75 g, more preferably 0.025-0.75 g, even more preferably 0.05-0.75 g, most preferably 0.05-0.50 g. This embodiment is, for example, particularly preferred if the animal is a bovine or a horse, more preferably wherein the animal is a calf or a foal, most preferably wherein the animal is a calf.

In an additional and/or even more preferred alternative embodiment, the sialylated MMO/first sialylated MMO is administered at a daily dose of 0.1-100.0 mg, preferably 0.1-75.0 mg, more preferably 0.5-75.0 mg, even more preferably 1.0-75.0 mg, even more preferably 2.5-75.0 mg, most preferably 2.5-50.0 mg, per kg bodyweight of the animal. This embodiment is, for example, particularly preferred if the animal is a bovine or a horse, more preferably wherein the animal is a calf or a foal, most preferably wherein the animal is a calf.

In an even more preferred alternative embodiment, the sialylated MMO/first sialylated MMO is administered at a daily dose of 0.01-5.0 g, preferably 0.1-2.5 g, more preferably 0.1-2.0 g, even more preferably 0.1-1.5 g, even more preferably 0.2-1.5 g, even more preferably 0.3-1.5 g, even more preferably 0.4-1.5 g, most preferably 0.5-1.5 g. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In an additional and/or even more preferred alternative embodiment, the sialylated MMO/first sialylated MM is administered at a daily dose of 0.1-150.0 mg, preferably 0.5-150.0 mg, more preferably 1.0-150.0 mg, even more preferably 1.0-125.0 mg, even more preferably 2.5-125.0 mg, even more preferably 5.0-125.0 mg, even more preferably 10.0-125.0 mg, even more preferably 25.0-125.0 mg, even more preferably 50.0-125.0 mg, most preferably 75.0-125.0 mg, per kg bodyweight of the animal. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In an even more preferred alternative embodiment, the sialylated MMO/first sialylated MMO is administered at a daily dose of 0.0001-1.0 g, preferably 0.0001-0.50 g, more preferably 0.0001-0.10 g, even more preferably 0.0001-0.075 g, even more preferably 0.0001-0.050 g, even more preferably 0.0002-0.050 g, even more preferably 0.0005-0.050 g, most preferably 0.0005-0.025 g. This embodiment is, for example, particularly preferred if the animal is a fish or a crustacean, more preferably a fish, even more preferably a fry or a fingerling, even more preferably a fingerling.

In an additional and/or even more preferred alternative embodiment, the sialylated MMO/first sialylated MMO is administered at a daily dose of 0.1-70.0 mg, preferably 0.1-60.0 mg, more preferably 0.1-50.0 mg, even more preferably 0.5-50.0 mg, even more preferably 1.0-50.0 mg, even more preferably 2.5-50.0 mg, even more preferably 5.0-50.0 mg, even more preferably 10.0-50.0 mg, most preferably 10.0-40.0 mg, per kg bodyweight of the animal. This embodiment is, for example, particularly preferred if the animal is a fish or a crustacean, more preferably a fish, even more preferably a fry or a fingerling, even more preferably a fingerling.

In an additional and/or even more preferred alternative embodiment, the second sialylated MMO is administered at a daily dose of 0.01-1.0 g, preferably 0.01-0.75 g, more preferably 0.025-0.75 g, even more preferably 0.025-0.50 g, most preferably 0.025-0.25 g. This embodiment is, for example, particularly preferred if the animal is a bovine or a horse, more preferably wherein the animal is a calf or a foal, most preferably wherein the animal is a calf.

In an additional and/or even more preferred alternative embodiment, the second sialylated MMO is administered at a daily dose of 0.1-100.0 mg, preferably 0.1-75.0 mg, more preferably 0.25-75.0 mg, even more preferably 0.5-75.0 mg, even more preferably 1.0-75.0 mg, even more preferably 1.0-50.0 mg, most preferably 1.0-25.0 mg, per kg bodyweight of the animal. This embodiment is, for example, particularly preferred if the animal is a bovine or a horse, more preferably wherein the animal is a calf or a foal, most preferably wherein the animal is a calf.

In an even more preferred alternative embodiment, the second sialylated MMO is administered at a daily dose of 0.01-5.0 g, preferably 0.1-2.5 g, more preferably 0.1-2.0 g, even more preferably 0.1-1.5 g, even more preferably 0.1-1.2 g, even more preferably 0.15-1.2 g, even more preferably 0.2-1.2 g, most preferably 0.25-1.2 g. This embodiment is, for example, particularly preferred if the animal is a pig, a sheep or a goat, more preferably wherein the animal is a piglet, a lamb or a kid, most preferably wherein the animal is a piglet.

In an additional and/or even more preferred alternative embodiment, the second sialylated MMO is administered at a daily dose of 0.1-150.0 mg, preferably 1.0-150.0 mg, more preferably 1.0-125.0 mg, even more preferably 2.5-125.0 mg, even more preferably 5.0-125.0 mg, even more preferably 10.0-125.0 mg, even more preferably 25.0-125.0 mg, even more preferably 25.0-100.0 mg, most preferably 25.0-80.0 mg, per kg bodyweight of the animal. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In an even more preferred alternative embodiment, the second sialylated MMO is administered at a daily dose of 0.0001-1.0 g, preferably 0.0001-0.50 g, more preferably 0.0001-0.10 g, even more preferably 0.0001-0.075 g, even more preferably 0.0001-0.050 g, even more preferably 0.0002-0.050 g, even more preferably 0.0002-0.030 g, most preferably 0.0002-0.020 g. This embodiment is, for example, particularly preferred if the animal is a fish or a crustacean, more preferably a fish, even more preferably a fry or a fingerling, even more preferably a fingerling.

In an additional and/or even more preferred alternative embodiment, the second sialylated MMO is administered at a daily dose of 0.1-70.0 mg, preferably 0.1-60.0 mg, more preferably 0.1-50.0 mg, even more preferably 0.5-50.0 mg, even more preferably 1.0-50.0 mg, even more preferably 2.5-50.0 mg, even more preferably 5.0-50.0 mg, even more preferably 5.0-40.0 mg, most preferably 5.0-30.0 mg, per kg bodyweight of the animal. This embodiment is, for example, particularly preferred if the animal is a fish or a crustacean, more preferably a fish, even more preferably a fry or a fingerling, even more preferably a fingerling.

In an even more preferred embodiment, the sialylated MMO/first sialylated MMO/second sialylated MMO is administered to the animal for at least 1 week, preferably at least 2 weeks, more preferably at least 3 weeks, even more preferably at least 4 weeks, most preferably at least 6 weeks.

In an additional and/or even more preferred alternative embodiment, the sialylated MMO/first sialylated MMO/second sialylated MMO is administered to the animal for ≤32 weeks, preferably ≤28 weeks, more preferably ≤24 weeks, even more preferably ≤20 weeks, even more preferably ≤16 weeks, most preferably ≤12 weeks.

In an additional and/or even more preferred alternative embodiment, the sialylated MMO/first sialylated MMO/second sialylated MMO is administered to the animal for 1-30, preferably 2-30, more preferably 3-30, even more preferably 4-30, even more preferably 5-30, even more preferably 6-30, even more preferably 6-24, even more preferably 6-18, most preferably 6-12, consecutive weeks.

In an additional and/or even more preferred alternative embodiment, the sialylated MMO/first sialylated MMO/second sialylated MMO is administered to the farm animal for:
if subject is bovine: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
if subject is a pig: 1-18, preferably 1-16, more preferably 1-14, even more preferably 1-12, even more preferably 2-12, even more preferably 3-12, even more preferably 4-12, most preferably 6-12, consecutive weeks;
if subject is a sheep: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
if subject is a goat: 1-18, preferably 1-16, more preferably 1-14, even more preferably 1-12, even more preferably 2-12, even more preferably 3-12, even more preferably 4-12, most preferably 6-12, consecutive weeks;
if subject is a horse: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
if subject is a camelid: 1-14, preferably 1-12, more preferably 2-12, even more preferably 3-12, even more preferably 4-12, most preferably 6-12, consecutive weeks;
if subject is a rabbit: 1-12, preferably 2-12, more preferably 3-12, even more preferably 4-12, most preferably 6-12, consecutive weeks;
if subject is a fish or a crustacean: 1-36, preferably 1-32, more preferably 1-24, even more preferably 1-18, even more preferably 1-12, even more preferably 2-12, even more preferably 3-12, even more preferably 6-12, consecutive months;
if subject is poultry (but not a turkey): 1-8, preferably 1-7, more preferably 2-7, even more preferably 3-7, most preferably 4-7, consecutive weeks; and
if subject is a turkey: 1-18, preferably 1-16, more preferably 1-14, even more preferably 1-12, even more preferably 2-12, even more preferably 3-12, even more preferably 4-12, most preferably 6-12, consecutive weeks.

Preferably, the sialylated MMO/first sialylated MMO/second sialylated MMO according to the disclosure is administered at least once a week (i.e., at least one daily dosage is administered in a week), more preferably at least once every 3 days, even more preferably at least once every 2 days, most preferably at least once daily (i.e., a daily dosage is administered every day).

In an even more preferred embodiment, the sialylated MMO/first sialylated MMO/second sialylated MMO is administered to the animal until the age of slaughtering.

When a fucosylated MMO according to the disclosure and a sialylated MMO (preferably a first sialylated MMO and a second sialylated MMO, optionally further sialylated MMOs; more preferably a first sialylated MMO and a second sialylated MMO) according to the disclosure (it is referred to in the section titled “Oligosaccharide”) are administered to a farm animal, it is preferred that the sialylated MMO is administered from the start, and preferably for at least 15.0%, more preferably for at least 20.0%, even more preferably for at least 25.0%, even more preferably for at least 40.0%, most preferably for at least 50.0% of the total period wherein the MMOs according to the disclosure are administered to the farm animal (it is in this context referred to in the section title “Method to reduce FCR”). The fucosylated MMO according to the disclosure is preferably administered to the farm animal after at least 0%, preferably at least 10%, more preferably at least 25%, even more preferably at least 30%, most preferably at least 50%, of the total period wherein MMOs are administered to the farm animal has passed.

When a fucosylated MMO according to the disclosure and a sialylated MMO (preferably a first sialylated MMO and a second sialylated MMO, optionally further sialylated MMOs; more preferably a first sialylated MMO and a second sialylated MMO) according to the disclosure (it is referred to in the section titled “Oligosaccharide”) are administered to a farm animal, it is particularly preferred to apply a two staged feeding regimen as described herein as it was surprisingly found that this regimen results in an optimal reduction in the feed conversion ratio (FCR) in a farm animal (likewise in an optimal average daily gain of the farm animal as disclosed in the third aspect of the disclosure). Surprisingly, it was found that a sialylated MMO (preferably a first sialylated MMO and a second sialylated MMO, optionally further sialylated MMOs; more preferably a first sialylated MMO and a second sialylated MMO) according to the disclosure primarily contributes to a decrease in FCR (likewise an increase in the average daily gain as disclosed in the third aspect of the disclosure) in a farm animal from the start of the feeding regimen (i.e., the total period wherein the MMOs according to the disclosure are administered to the farm animal; it is also referred to in the section titled “Method to reduce FCR”) until ≤75.0%, preferably ≤50.0% of the feeding regimen has passed. A fucosylated MMO according to the disclosure primarily contributes to a decrease in FCR (likewise in an increase in the average daily gain as disclosed in the third aspect of the disclosure) in a farm animal not earlier than after 20.0%, preferably 25.0%, more preferably 50.0%, of the feeding regimen (i.e., the total period wherein the MMOs according to the disclosure are administered to the farm animal) has passed.

In other words, surprisingly, it was found that a feeding regimen with a fucosylated MMO according to the disclosure and a sialylated MMO (preferably a first sialylated MMO and a second sialylated MMO, optionally further sialylated MMOs; more preferably a first sialylated MMO and a second sialylated MMO) according to the disclosure is more preferably split in two different stages:
stage 1: administering a sialylated MMO (preferably a first sialylated MMO and a second sialylated MMO, optionally further sialylated MMOs; more preferably a first sialylated MMO and a second sialylated MMO) according to the disclosure and optionally administering a fucosylated MMO according to the disclosure, preferably administering a sialylated MMO (preferably a first sialylated MMO and a second sialylated MMO, optionally further sialylated MMOs; more preferably a first sialylated MMO and a second sialylated MMO) according to the disclosure without administering a fucosylated MMO according to the disclosure; and
stage 2: administering a fucosylated MMO according to the disclosure and optionally administering a sialylated MMO according to the disclosure, preferably administering a fucosylated MMO according to the disclosure without administering a sialylated MMO according to the disclosure;
even more preferably split in two different stages:
stage 1: administering a sialylated MMO (preferably a first sialylated MMO and a second sialylated MMO, optionally further sialylated MMOs; more preferably a first sialylated MMO and a second sialylated MMO) according to the disclosure and a fucosylated MMO according to the disclosure; and
stage 2: administering a fucosylated MMO according to the disclosure without administering a sialylated MMO according to the disclosure.

For the sake of clarity, in the context of the disclosure, “different” stages refer to stages being different in the MMOs administered to the subject. If, in the first stage, a sialylated MMO is administered according to the disclosure, then the second stage has to be either the administration of a fucosylated MMO according to the disclosure or the administration of a fucosylated MMO according to the disclosure and a sialylated MMO according to the disclosure. If, in the first stage, a sialylated MMO according to the disclosure and a fucosylated MMO according to the disclosure are administered to the subject, then the second stage has to be either the administration of a fucosylated MMO according to the disclosure or the administration of a sialylated MMO according to the disclosure.

As understood by the skilled person, stage 1 precedes stage 2 and stage 1 is the start of the total period wherein the MMO(s) according to the disclosure are administered to the farm animal (it is also referred to in the section titled “Method to reduce FCR”). In this context, it is a preferred embodiment that the stage 1 constitutes at least 15.0%, preferably at least 20.0%, more preferably at least 25.0%, even more preferably at least 30.0%, even more preferably at least 35.0%, even more preferably at least 40.0%, even more preferably at least 45.0%, most preferably at least 50.0%, of the of the total period wherein the MMO(s) according to the disclosure are administered to the farm animal as described in the present application (it is also referred to in the section titled “Method to reduce FCR”). In an additional and/or preferred alternative embodiment, the stage 1 constitutes ≤80.0%, preferably ≤75.0%, more preferably ≤70.0%, even more preferably ≤⁶5.0%, even more preferably ≤60.0%, even more preferably ≤55.0%, most preferably ≤50.0%, of the of the total period wherein the MMO(s) according to the disclosure are administered to the farm animal as described in the present application (it is also referred to in the section titled “Method to reduce FCR”). It is a more preferred embodiment that the stage 1 constitutes 15.0-80.0%, preferably 15.0-75.0%, more preferably 20.0-75.0%, of the of the total period wherein the MMO(s) according to the disclosure are administered to the farm animal as described in the present application (it is also referred to in the section titled “Method to reduce FCR”). If the farm animal is a rearing calf, then it is particularly preferred that stage 1 constitutes 35.0-75.0%, more preferably 40.0-75.0%, most preferably 50.0-75.0%, of the of the total period wherein the MMO(s) according to the disclosure are administered to the farm animal as described in the present application (it is also referred to in the section titled “Method to reduce FCR”). If the farm animal is a veal calf, then it is particularly preferred that stage 1 constitutes 15.0-50.0%, more preferably 15.0-40.0%, most preferably 15.0-30.0%, of the of the total period wherein the MMO(s) according to the disclosure are administered to the farm animal as described in the present application (it is also referred to in the section titled “Method to reduce FCR”).

It is another preferred embodiment, that stage 1 constitutes at least 2 weeks, preferably at least 3 weeks, more preferably at least 4 weeks, even more preferably at least 5 weeks, most preferably at least 6 weeks. In an additional and/or preferred alternative embodiment, the stage 1 constitutes ≤10 weeks, preferably ≤9 weeks, more preferably ≤8 weeks, even more preferably ≤7 weeks, most preferably ≤6 weeks. It is a more preferred embodiment that the stage 1 constitutes 2-10 weeks, preferably 2-8 weeks, more preferably 4-8 weeks, even more preferably 4-6 weeks, most preferably 6 weeks.

In this context, it is a preferred embodiment that the stage 2 constitutes at least 20.0%, preferably at least 25.0%, more preferably at least 30.0%, even more preferably at least 35.0%, even more preferably at least 40.0%, even more preferably at least 45.0%, most preferably at least 50.0%, of the of the total period wherein the MMO(s) according to the disclosure are administered to the farm animal as described in the present application (it is also referred to in the section titled “Method to reduce FCR”). In an additional and/or preferred alternative embodiment, the stage 2 constitutes ≤85.0%, preferably ≤80.0%, more preferably ≤75.0%, even more preferably ≤70.0%, even more preferably ≤65.0%, even more preferably ≤60.0%, even more preferably ≤55.0%, most preferably ≤50.0%, of the of the total period wherein the MMO(s) according to the disclosure are administered to the farm animal as described in the present application (it is also referred to in the section titled “Method to reduce FCR”). It is a more preferred embodiment that the stage 2 constitutes 20.0-85.0%, preferably 25.0-85.0%, more preferably 25.0-80.0%, of the of the total period wherein the MMO(s) according to the disclosure are administered to the farm animal as described in the present application (it is also referred to in the section titled “Method to reduce FCR”). If the farm animal is a rearing calf, then it is particularly preferred that stage 1 constitutes 25.0-65.0%, more preferably 25.0-60.0%, most preferably 25.0-50.0%, of the of the total period wherein the MMO(s) according to the disclosure are administered to the farm animal as described in the present application (it is also referred to in the section titled “Method to reduce FCR”). If the farm animal is a veal calf, then it is particularly preferred that stage 1 constitutes 50.0-85.0%, more preferably 60.0-85.0%, most preferably 70.0-85.0%, of the of the total period wherein the MMO(s) according to the disclosure are administered to the farm animal as described in the present application (it is also referred to in the section titled “Method to reduce FCR”).

It is another preferred embodiment, that stage 2 constitutes at least 2 weeks, preferably at least 3 weeks, more preferably at least 4 weeks, even more preferably at least 5 weeks, most preferably at least 6 weeks. In an additional and/or preferred alternative embodiment, the stage 1 constitutes ≤22 weeks, preferably ≤20 weeks. If the farm animal is a rearing calf, then it is particularly preferred that stage 2 constitutes 2-6 weeks, preferably 2-5 weeks, more preferably 3-5 weeks, even more preferably 3-4 weeks, most preferably 3 weeks. If the farm animal is a veal calf, then it is particularly preferred that stage 2 constitutes 6-22 weeks, preferably 8-22 weeks, more preferably 12-22 weeks, even more preferably 12-20 weeks, most preferably 20 weeks.

For the sake of clarity, if, for example, a farm animal was administered 3′SL and 6′SL for 6 weeks, followed by administration of 2′FL for the 6 subsequent weeks, then it can be concluded that (i) the total period wherein MMO(s) are administered is 12 weeks, (ii) the sialylated MMOs (i.e., 3′SL and 6′SL) were administered from the start (i.e., day 1 of the method of the disclosure), (iii) the sialylated MMOs (i.e., 3′SL and 6′SL) were administered for 50% of the total period wherein MMO(s) are administered, i.e., 6 weeks, (iv) the fucosylated MMO (i.e., 2′FL) were administered after 50% of the total period wherein MMO(s) were administered has passed, i.e., the administration of the fucosylated MMO started after 6 weeks (and not earlier), (v) the fucosylated MMO (i.e., 2′FL) was administered to the end of the total period, i.e., administered to the end of week 12, and (vi) the fucosylated MMO (i.e., 2′FL) was administered for 50% of the total period wherein MMO(s) are administered, i.e., 6 weeks. Further, as elaborated herein, administration of 2′FL can be at least once a week, preferably at least once every 3 days, more preferably at least once every 2 days, most preferably at least once daily. Similarly, as elaborated herein, administration of 3′SL and 6′SL can be at least once a week, preferably at least once every 3 days, more preferably at least once every 2 days, most preferably at least once daily.

If, in another example, a farm animal was administered 2′FL for 6 weeks, followed by administration of 3′SL and 6′SL for the 6 subsequent weeks, then it can be concluded that (i) the total period wherein MMO(s) are administered is 12 weeks, (ii) the fucosylated MMO (i.e., 2′FL) was administered from the start (i.e., day 1 of the method of the disclosure), (iii) the fucosylated MMO (i.e., 2′FL) was administered for 50% of the total period wherein MMO(s) are administered, i.e., 6 weeks, (iv) the sialylated MMO (i.e., 3′SL and 6′SL) were administered after 50% of the total period wherein MMO(s) were administered has passed, i.e., the administration of the sialylated MMO started after 6 weeks (and not earlier), (v) the sialylated MMO (i.e., 3′SL and 6′SL) were administered to the end of the total period, i.e., administered to the end of week 12, and (vi) the sialylated MMO (i.e., 3′SL and 6′SL) were administered for 50% of the total period wherein MMO(s) are administered, i.e., 6 weeks. Further, as elaborated herein, administration of 2′FL can be at least once a week, preferably at least once every 3 days, more preferably at least once every 2 days, most preferably at least once daily. Similarly, as elaborated herein, administration of 3′SL and 6′SL can be at least once a week, preferably at least once every 3 days, more preferably at least once every 2 days, most preferably at least once daily.

### Composition: General

In an embodiment of the first aspect of the disclosure, the method reduces the feed conversion ratio (FCR) of a farm animal, wherein the method comprises the step of administering to the farm animal: (i) a fucosylated MMO, (ii) a sialylated MMO, or (iii) a fucosylated MMO and a sialylated MMO as described herein.

In a preferred embodiment, the fucosylated MMO (it is referred to in the section titled “Oligosaccharide”) is part of a composition, preferably a composition as described in the present section titled “Composition.”

In an additional and/or preferred alternative embodiment, the sialylated MMO (it is referred to in the section titled “Oligosaccharide”) is part of a composition, preferably a composition as described in the present section titled “Composition.”

In an additional and/or preferred alternative embodiment, the first sialylated MMO (it is referred to in the section titled “Oligosaccharide”) is part of a composition, preferably a composition as described in the present section titled “Composition.”

In an additional and/or preferred alternative embodiment, the first sialylated MMO and the second sialylated MMO (it is referred to in the section titled “Oligosaccharide”) are part of a composition, preferably a composition as described in the present section titled “Composition.”

In the context of the disclosure, each MMO according to the disclosure that is administered to the farm animal in a method according to the disclosure, are preferably part of the same composition, preferably a composition as described in the present section titled “Composition.”

Hence, in a more preferred embodiment, the disclosure provides a method to reduce the feed conversion ratio (FCR) of a farm animal, wherein the method comprises the step of administering to the farm animal: (i) a fucosylated mammalian milk oligosaccharide (MMO), (ii) a sialylated MMO, or (iii) a fucosylated MMO and a sialylated MMO; wherein the (i), (ii) and/or (iii) are part of a composition, preferably a composition as described in the present section titled “Composition” and preferably are part of the same composition. In other words, in a more preferred embodiment, the disclosure provides a method to reduce the feed conversion ratio (FCR) of a farm animal, wherein the method comprises the step of administering a composition, preferably a composition as described in the present section titled “Composition,” to the farm animal, wherein the composition comprises (i) a fucosylated mammalian milk oligosaccharide (MMO), (ii) a sialylated MMO, or (iii) a fucosylated MMO and a sialylated MMO.

The “farm animal” is as described in the section titled “Subject.” The “method to reduce FCR” is as described in the section titled “Method to reduce FCR.” The “fucosylated MMO,” “sialylated MMO” and “fucosylated MMO and a sialylated MMO” are as described in the section titled “Oligosaccharide.”

Hence, in another more preferred embodiment, the disclosure provides a method to reduce the feed conversion ratio (FCR) of a farm animal, wherein the method comprises the step of administering to the farm animal: (i) a fucosylated mammalian milk oligosaccharide (MMO), (ii) a sialylated MMO, or (iii) a fucosylated MMO and a sialylated MMO; wherein the (i), (ii) or (iii) reduces the FCR of the farm animal and wherein the (i), (ii) and/or (iii) are part of a composition, preferably a composition as described in the present section titled “Composition” and preferably are part of the same composition. In other words, in another more preferred embodiment, the disclosure provides a method to reduce the feed conversion ratio (FCR) of a farm animal, wherein the method comprises the step of administering a composition, preferably a composition as described in the present section titled “Composition,” to the farm animal, wherein the composition comprises (i) a fucosylated mammalian milk oligosaccharide (MMO), (ii) a sialylated MMO, or (iii) a fucosylated MMO and a sialylated MMO, and wherein the (i), (ii) or (iii) reduces the FCR of the farm animal.

The “farm animal” is as described in the section titled “Subject.” The “method to reduce FCR” is as described in the section titled “Method to reduce FCR.” The “fucosylated MMO,” “sialylated MMO” and “fucosylated MMO and a sialylated MMO” are as described in the section titled “Oligosaccharide.”

Hence, in another more preferred embodiment, the disclosure provides a method of feeding a farm animal, wherein the method comprises the step of administering to the farm animal: (i) a fucosylated mammalian milk oligosaccharide (MMO), (ii) a sialylated MMO, or (iii) a fucosylated MMO and a sialylated MMO; wherein the (i), (ii) or (iii) reduces the FCR of the farm animal and wherein the (i), (ii) and/or (iii) are part of a composition, preferably a composition as described in the present section titled “Composition” and preferably are part of the same composition. In other words, in another more preferred embodiment, the disclosure provides a method of feeding a farm animal, wherein the method comprises the step of administering a composition, preferably a composition as described in the present section titled “Composition,” to the farm animal, wherein the composition comprises (i) a fucosylated mammalian milk oligosaccharide (MMO), (ii) a sialylated MMO, or (iii) a fucosylated MMO and a sialylated MMO, and wherein the (i), (ii) or (iii) reduces the FCR of the farm animal.

The “farm animal” is as described in the section titled “Subject.” The “method to reduce FCR” is as described in the section titled “Method to reduce FCR.” The “fucosylated MMO,” “sialylated MMO” and “fucosylated MMO and a sialylated MMO” are as described in the section titled “Oligosaccharide.”

In a preferred embodiment, the composition according to the disclosure is not mother's milk.

In an additional and/or preferred alternative embodiment, the composition according to the disclosure is a synthetic composition.

In the context of the disclosure, a “synthetic composition” or a “synthetic nutritional composition” refers to a composition that is artificially prepared and preferably refers to a composition comprising at least one component that is produced ex vivo, either chemically and/or biologically, e.g., by means of chemical reaction, enzymatic reaction or recombinantly, or purified by humans. It is preferred that a synthetic composition of the disclosure is not identical with a naturally occurring composition.

In this context, it is preferred that the fucosylated MMO and/or the sialylated MMO, preferably all MMOs according to the disclosure as described herein, has been isolated from a microbial cultivation or fermentation, cell culture, enzymatic reaction or chemical reaction or has been isolated from a natural source (it is referred to in the section titled “Oligosaccharide”). It is more preferred that the fucosylated MMO and/or the sialylated MMO, preferably all MMOs according to the disclosure as described herein, has been produced in vitro and/or ex vivo by a cell (it is referred to in the section titled “Oligosaccharide”).

In an additional and/or preferred alternative embodiment, the composition according to the disclosure further comprises a microbial strain that does not use, preferably is not able to use, the fucosylated MMO and/or the sialylated MMO, preferably is not able to use any of the MMOs according to the disclosure, as a carbon source.

In a preferred alternative embodiment, the composition according to the disclosure does not comprise a microbial strain. Examples of such a strain is Bifidobacterium, Lactobacillus and Saccharomyces boulardii.

In a preferred embodiment, the composition according to the disclosure is a pharmaceutical composition, optionally further comprising a pharmaceutically acceptable carrier, filler, preservative, solubilizer, diluent, excipient, salt, adjuvant and/or solvent.

In an additional and/or preferred alternative embodiment, the composition is a nutritional composition, optionally further comprising a feed ingredient and/or a food ingredient, wherein the feed/food ingredient is preferably chosen from the group consisting of:
a lipid, preferably one or more selected from the group consisting of an oil, fat, ester, monoglyceride, diglyceride, triglyceride and free fatty acid;
a vitamin, preferably one or more selected from the group consisting of vitamin A, vitamin B, vitamin C, vitamin D, vitamin E and vitamin H, or a derivate thereof;
an amino acid compound;
a trace element;
a mineral, preferably one or more selected from the group consisting of calcium, phosphorus, magnesium, iron, zinc, manganese, copper, sodium, potassium, molybdenum, chromium, selenium and chloride;
an antioxidant;
a prebiotic agent, preferably one or more selected from the group consisting of GOS (galacto-oligosaccharide), FOS (fructo-oligosaccharide), inulin and resistant starch;
a carbohydrate;
an antimicrobial agent; and/or
a protein.

Generally, any source of protein may be used so long as it is suitable for nutritional compositions and is otherwise compatible with any other selected ingredients or features in the nutritional composition. Non-limiting examples of suitable proteins (and sources thereof) suitable for use in the nutritional composition according to the disclosure include, but are not limited to, intact, hydrolyzed, or partially hydrolyzed protein, which may be derived from any known or otherwise suitable source such as milk (e.g., casein, whey), animal (e.g., meat, fish), cereal (e.g., rice, corn, wheat), vegetable (e.g., soy, pea, potato, bean), and combinations thereof. The protein may also include a mixture of amino acids (often described as free amino acids) known for use in nutritional products or a combination of such amino acids with the intact, hydrolyzed, or partially hydrolyzed proteins described herein. The amino acids may be naturally occurring or synthetic amino acids. More particular examples of suitable protein (or sources thereof) used in a nutritional composition according to the disclosure include, but are not limited to, whole cow's milk, partially or completely defatted milk, milk protein concentrates, milk protein isolates, nonfat dry milk, condensed skim milk, whey protein concentrates, whey protein isolates, acid caseins, sodium caseinates, calcium caseinates, potassium caseinates, legume protein, soy protein concentrates, soy protein isolates, pea protein concentrates, pea protein isolates, collagen proteins, potato proteins, rice proteins, wheat proteins, canola proteins, quinoa, insect proteins, earthworm proteins, fungal (e.g., mushroom) proteins, hydrolyzed yeast, gelatin, bovine colostrum, human colostrum, glycol macropeptides, mycoproteins, proteins expressed by microorganisms (e.g., bacteria and algae), and combinations thereof. A nutritional composition according to the disclosure may include any individual source of protein or combination of the various sources of protein grouped above. In addition, the proteins for use herein can also include, or be entirely or partially replaced by, free amino acids known for use in nutritional products, non-limiting examples of which include L-tryptophan, L-glutamine, L-tyrosine, L-methionine, L-cysteine, taurine, L-arginine, L-carnitine, and combinations thereof.

The carbohydrate or source of carbohydrate suitable for use in a nutritional composition according to the disclosure may be simple, complex, or variations or combinations thereof. Generally, the carbohydrate may include any carbohydrate or carbohydrate source that is suitable for use in nutritional compositions and is otherwise compatible with any other selected ingredients or features in the nutritional composition. Non-limiting examples of carbohydrates suitable for use in the nutritional composition according to the disclosure, but are not limited to, polydextrose, maltodextrin; hydrolyzed or modified starch or cornstarch; glucose polymers; corn syrup; corn syrup solids; sucrose; glucose; fructose; lactose; high fructose corn syrup; honey; sugar alcohols (e.g., maltitol, erythritol, sorbitol); isomaltulose; sucromalt; pullulan; potato starch; and other slowly-digested carbohydrates; dietary fibers including, but not limited to, fructooligosaccharides (FOS), galactooligosaccharides (GOS), oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinogalactans, glucomannan, xanthan gum, alginate, pectin, low methoxy pectin, high methoxy pectin, cereal beta-glucans (e.g., oat beta-glucan, barley beta-glucan), carrageenan and psyllium, soluble and insoluble fibers derived from fruits or vegetables; other resistant starches; and combinations thereof. A nutritional composition according to the disclosure may include any individual source of carbohydrate or combination of the various sources of carbohydrate listed above.

The fat or source of fat suitable for use in a nutritional composition according to the disclosure may be derived from various sources including, but not limited to, plants, animals, and combinations thereof. Generally, the fat may include any fat or fat source that is suitable for use in a nutritional composition according to the disclosure and is otherwise compatible with any other selected ingredients or features in the nutritional composition. Non-limiting examples of suitable fat (or sources thereof) for use in a nutritional composition according to the disclosure include coconut oil, fractionated coconut oil, soy oil, high oleic soy oil, corn oil, olive oil, safflower oil, high oleic safflower oil, medium chain triglyceride oil (MCT oil), high gamma linolenic (GLA) safflower oil, sunflower oil, high oleic sunflower oil, palm oil, palm kernel oil, palm olein, canola oil, high oleic canola oil, marine oils, fish oils, algal oils, borage oil, cottonseed oil, fungal oils, eicosapentaenoic acid (EPA), docosahexaenoic acid (DHA), arachidonic acid (ARA), conjugated linoleic acid (CLA), α-linolenic acid, rice bran oil, wheat bran oil, interesterified oils, transesterified oils, structured lipids, and combinations thereof. Generally, the fats used in a nutritional composition for formulating infant formulas and pediatric formulas provide fatty acids needed both as an energy source and for the healthy development of the infant, toddler, or child. These fats typically comprise triglycerides, although the fats may also comprise diglycerides, monoglycerides, and free fatty acids. Fatty acids provided by the fats in the nutritional composition include, but are not limited to, capric acid, lauric acid, myristic acid, palmitic acid, palmitoleic acid, stearic acid, oleic acid, linoleic acid, α-linolenic acid, ARA, EPA, and DHA. The nutritional composition can include any individual source of fat or combination of the various sources listed above. Preferably, the fat is a mixture of vegetable fat and milk fat such as obtained from milk from a mammal like cow, sheep, goat, mare, or camel. More preferably, wherein the milk fat is bovine milk fat. Mixtures of different types of fat are preferred because they help to provide different fatty acids and better resemble the type of linkage between the glycerol moiety and the fatty acid moiety in the fat, when compared to human mother's milk.

When one or more prebiotics is/are present, then it is preferred that the weight ratio between the oligosaccharide according to the disclosure and the prebiotic(s) is in the range of from 0.5:10 to 10:0.5.

Preferably, the pharmaceutical or nutritional composition according to the disclosure comprises one or more probiotics for its beneficial effect on the subject's gut microbiome. Examples of probiotics include Bifidobacterium, Lactobacillus and Saccharomyces boulardii.

In a more preferred embodiment, the nutritional composition is a synthetic nutritional composition.

In an additional and/or more preferred alternative embodiment, the nutritional composition is selected from a group consisting of a milk replacer, creep feed, prestarter diet, starter diet, pre-weaning feed, weaning feed and post-weaning feed, preferably selected form the group consisting of a milk replacer, starter diet, creep feed and post-weaning feed, more preferably the nutritional composition is a milk replacer or starter diet, most preferably a milk replacer.

In the context of the disclosure, the term “creep feed” refers to a nutritional composition that is used to supplement a pre-weaned animal with a solid diet while the animal is suckling. For example, a piglet receives creep feed to ease the transition from sow's milk to solid pig starter feed. The creep feed stimulates the digestive system of the piglet to produce, e.g., amylase, which digests carbohydrates in dry feed. If a pig is better able to digest dry feed, he can start eating quickly post-weaning for better performance.

In the context of the disclosure, the term “milk replacer” refers to a nutritional composition that serves as a substitute for mother's milk. A milk replacer comprises milk proteins, fat, carbohydrates, vitamins and minerals. Preferably, a milk replacer comprises 10 dry wt. % to 28 dry wt. % of at least one protein, 13 dry wt. % to 28 dry wt. % (preferably 20 dry wt. % to 25 dry wt. %) of at least one fat and lactose at <55 dry wt. %. More preferably, a milk replacer comprises 18 dry wt. % to 24 dry wt. % of at least one protein, 15 dry wt. % to 28 dry wt. % (preferably 20 dry wt. % to 25 dry wt. %) of at least one fat and lactose at <50 dry wt. %. In this context, “dry wt. %” refers to the ratio between the weight of the recited component (e.g., 2′FL) and the dry weight of the milk replacer (w/v %). Throughout the application and claims, a milk replacer is preferably a calf milk replacer.

Surprisingly, it was found that administering (i) a fucosylated MMO, (ii) a sialylated MMO or (iii) a fucosylated MMO and a sialylated MMO according to the disclosure is sufficient to obtain an effective decrease of the FCR of a farm animal as described herein. Administering other (oligo)saccharides is not required to obtain the FCR reduction. While a composition according to the disclosure as described in the section titled “Composition” can include additional (oligo)saccharides, the disclosure relates to the fact that only (i) a fucosylated MMO, (ii) a sialylated MMO or (iii) a fucosylated MMO and a sialylated MMO according to the disclosure is required for achieving an efficient reduction of the feed conversion ratio as described herein.

In this context, it is hence a preferred embodiment that the composition according to the disclosure does not comprise lacto-N-tetraose (LNT). Likewise, it is a preferred embodiment that the method according to the disclosure does not comprise the administration of lacto-N-tetraose (LNT).

In an additional and/or preferred alternative embodiment, the composition according to the disclosure does not comprise lacto-N-neotetraose (LNnT). Likewise, it is a preferred embodiment that the method according to the disclosure does not comprise the administration of lacto-N-neotetraose (LNnT).

In an additional and/or preferred alternative embodiment, the composition according to the disclosure does not comprise N-acetyllactosamine. Likewise, it is a preferred embodiment that the method according to the disclosure does not comprise the administration of N-acetyllactosamine.

In an additional and/or preferred alternative embodiment, the composition according to the disclosure does not comprise an oligosaccharide containing N-acetyllactosamine, preferably does not comprise a saccharide containing N-acetyllactosamine. Likewise, it is a preferred embodiment that the method according to the disclosure does not comprise the administration of an oligosaccharide containing N-acetyllactosamine, preferably does not comprise the administration of a saccharide containing N-acetyllactosamine.

In an additional and/or preferred alternative embodiment, the composition according to the disclosure does not comprise a neutral non-fucosylated MMO.

In an additional and/or preferred alternative embodiment, the composition according to the disclosure does not comprise a fructo-oligosaccharide (FOS). Likewise, it is a preferred embodiment that the method according to the disclosure does not comprise the administration of a fructo-oligosaccharide (FOS).

In an additional and/or preferred alternative embodiment, the composition according to the disclosure does not comprise a galactose-oligosaccharide (GOS). Likewise, it is a preferred embodiment that the method according to the disclosure does not comprise the administration of a galactose-oligosaccharide (GOS).

In an additional and/or preferred alternative embodiment, the composition according to the disclosure does not comprise an isomaltooligosaccharide. Likewise, it is a preferred embodiment that the method according to the disclosure does not comprise the administration of isomaltooligosaccharide.

In an even more preferred embodiment, the composition according to the disclosure comprises only (i) a fucosylated MMO, (ii) a sialylated MMO or (iii) a fucosylated MMO and a sialylated MMO according to the disclosure (it is referred to in the section titled “Oligosaccharide”). Likewise, it is a more preferred embodiment that the method according to the disclosure only comprises the administration of (i) a fucosylated MMO, (ii) a sialylated MMO or (iii) a fucosylated MMO and a sialylated MMO according to the disclosure (it is referred to in the section titled “Oligosaccharide”).

In an additional and/or even more preferred alternative embodiment, the composition according to the disclosure does not comprise a neutral non-fucosylated MMO.

### Composition: Fucosylated MMO

In a preferred embodiment, the fucosylated mammalian milk oligosaccharide (MMO) is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.

Throughout the application and claims, unless specifically stated otherwise, the term “wt. %” means weight by weight (i.e., w/w). If the composition is, for example, a solid composition, then 1.0 wt. % means 1.0 g fucosylated MMO per 100.0 gram of the solid composition. If the composition is, for example, a liquid composition, then 1.0 wt. % means 1.0 g fucosylated MMO per 100.0 gram of the liquid composition.

Throughout the application and claims, if the composition is a milk replacer (e.g., calf milk replacer) then the wt. % as recited throughout the application and claims, refers to the dry wt. %. If the composition is a (calf) milk replacer, then, for example, aforementioned paragraph should be read as: “In a preferred embodiment, the fucosylated mammalian milk oligosaccharide (MMO) is present at 0.001-1.000 dry wt. %, preferably 0.001-0.750 dry wt. %, more preferably 0.001-0.500 dry wt. %, even more preferably 0.001-0.400 dry wt. %, even more preferably 0.005-0.400 dry wt. %, even more preferably 0.005-0.300 dry wt. %, even more preferably 0.005-0.250 dry wt. %, most preferably 0.005-0.100 dry wt. %.”

Further, a milk replacer (e.g., calf milk replacer) is often administered to, e.g., a calf in a liquid form. Hence, in this context, it is preferred that a liquid milk replacer comprises 10.0-30.0% (w/v), more preferably 12.0-28.0% (w/v), milk replacer powder. Here, it is preferred that the milk replacer powder is constituted in water to obtain the liquid milk replacer. Throughout the application and claims, the term “% (w/v)” refers to the weight of the dissolved substance in gram (e.g., milk replacer powder) per 100 milliliters of the final solution (e.g., liquid milk replacer). If the milk replacer is a calf milk replacer, then it is particularly preferred that a liquid calf milk replacer comprises 10.0-20.0% (w/v), more preferably 12.0-20.0% (w/v), even more preferably 12.0-18.0% (w/v), even more preferably 14.0-18.0% (w/v), most preferably 15.0% (w/v), calf milk replacer powder. It is preferred that the calf milk replacer powder is constituted in water to obtain the liquid calf milk replacer. If the milk replacer is a milk replacer for a piglet, then it is particularly preferred that a liquid milk replacer comprises 10.0-30.0% (w/v), more preferably 15.0-30.0% (w/v), even more preferably 20.0-30.0% (w/v), even more preferably 22.0-28.0% (w/v), most preferably 25.0% (w/v), milk replacer powder. It is preferred that the milk replacer powder is constituted in water to obtain the liquid calf milk replacer.

In a more preferred embodiment, the fucosylated MMO is present at 0.001-0.250 wt. %, preferably 0.001-0.200 wt. %, more preferably 0.001-0.100 wt. %, even more preferably 0.001-0.020 wt. %, even more preferably 0.001-0.010 wt. %, even more preferably 0.002-0.010 wt. %, even more preferably 0.003-0.010 wt. %, even more preferably 0.003-0.0075 wt. %, most preferably 0.003-0.006 wt. %, of the total weight of the composition. This embodiment is, for example, particularly preferred if the animal is a bovine or a horse, more preferably wherein the animal is a calf or a foal, most preferably wherein the animal is a calf.

In a more preferred alternative embodiment, the fucosylated MMO is present at 0.010-1.000 wt. %, preferably 0.010-0.750 wt. %, more preferably 0.010-0.500 wt. %, even more preferably 0.010-0.400 wt. %, even more preferably 0.050-0.400 wt. %, even more preferably 0.100-0.400 wt. %, most preferably 0.100-0.300 wt. %, of the total weight of the composition. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In a more preferred alternative embodiment, the fucosylated MMO is present at 0.001-2.500 wt. %, preferably 0.001-1.000 wt. %, more preferably 0.010-1.000 wt. %, even more preferably 0.025-1.000 wt. %, even more preferably 0.025-0.750 wt. %, even more preferably 0.025-0.500 wt. %, even more preferably 0.025-0.250 wt. %, even more preferably 0.025-0.100 wt. %, most preferably 0.025-0.075 wt. %, of the total weight of the composition. This embodiment is, for example, particularly preferred if the animal is a fish or a crustacean, more preferably a fish, even more preferably a fry or a fingerling, even more preferably a fingerling.

In an additional and/or more preferred alternative embodiment, the fucosylated MMO according to the disclosure is provided as a powder in the composition according to the disclosure. Preferably, the powder is as defined earlier in the present section titled “Oligosaccharide.”

### Composition: Sialylated MMO

In a preferred embodiment, the sialylated mammalian milk oligosaccharide (MMO) is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.

In a more preferred embodiment, the sialylated MMO is present at 0.001-0.250 wt. %, preferably 0.001-0.200 wt. %, more preferably 0.001-0.100 wt. %, even more preferably 0.005-0.100 wt. %, even more preferably 0.005-0.075 wt. %, even more preferably 0.005-0.050 wt. %, even more preferably 0.005-0.040 wt. %, most preferably 0.010-0.040 wt. %, of the total weight of the composition. This embodiment is, for example, particularly preferred if the animal is a bovine or a horse, more preferably wherein the animal is a calf or a foal, most preferably wherein the animal is a calf.

In a more preferred alternative embodiment, the sialylated MMO is present at 0.010-1.000 wt. %, preferably 0.010-0.750 wt. %, even more preferably 0.010-0.750 wt. %, even more preferably 0.010-0.500 wt. %, even more preferably 0.025-0.500 wt. %, most preferably 0.050-0.500 wt. %, of the total weight of the composition. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In a more preferred alternative embodiment, the sialylated MMO is present at 0.001-2.500 wt. %, preferably 0.001-1.000 wt. %, more preferably 0.010-1.000 wt. %, even more preferably 0.025-1.000 wt. %, even more preferably 0.025-0.750 wt. %, even more preferably 0.025-0.500 wt. %, even more preferably 0.025-0.500 wt. %, even more preferably 0.025-0.400 wt. %, most preferably 0.050-0.400 wt. %, of the total weight of the composition. This embodiment is, for example, particularly preferred if the animal is a fish or a crustacean, more preferably a fish, even more preferably a fry or a fingerling, even more preferably a fingerling.

In an additional and/or more preferred alternative embodiment, the sialylated MMO according to the disclosure is provided as a powder in the composition according to the disclosure. Preferably, the powder is as defined earlier in the present section titled “Composition.”

In an even more preferred embodiment, the sialylated mammalian milk oligosaccharide (MMO) is present at 0.001-0.250 wt. %, preferably 0.001-0.200 wt. %, more preferably 0.001-0.100 wt. %, even more preferably 0.001-0.050 wt. %, even more preferably 0.005-0.050 wt. %, even more preferably 0.010-0.050 wt. %, even more preferably 0.015-0.050 wt. %, most preferably 0.015-0.035 wt. %, of the total weight of the composition. This embodiment is, for example, particularly preferred if the animal is a bovine or a horse, more preferably wherein the animal is a calf or a foal, most preferably wherein the animal is a calf.

In an even more preferred alternative embodiment, the sialylated MMO is present at 0.010-1.000 wt. %, preferably 0.050-0.750 wt. %, even more preferably 0.050-0.750 wt. %, even more preferably 0.050-0.500 wt. %, most preferably 0.100-0.500 wt. %, of the total weight of the composition. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In an even more preferred alternative embodiment, the sialylated MMO is present at 0.001-2.500 wt. %, preferably 0.001-1.000 wt. %, more preferably 0.010-1.000 wt. %, even more preferably 0.010-0.500 wt. %, even more preferably 0.015-0.500 wt. %, most preferably 0.015-0.350 wt. %, of the total weight of the composition. This embodiment is, for example, particularly preferred if the animal is a fish or a crustacean, more preferably a fish, even more preferably a fry or a fingerling, even more preferably a fingerling.

As elaborated in the section titled “Oligosaccharide,” it is preferred that two or more sialylated MMOs (i.e., a first sialylated MMO and a second sialylated MMO, optionally further sialylated MMOs are administered), more preferably two sialylated MMOs (i.e., a first sialylated MMO and a second sialylated MMO are administered), according to the disclosure are administered to the farm animal.

In this context, it is a preferred embodiment, that the first sialylated mammalian milk oligosaccharide (MMO) is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.

In a more preferred embodiment, the first sialylated MMO is present at 0.001-0.250 wt. %, preferably 0.001-0.200 wt. %, more preferably 0.001-0.100 wt. %, even more preferably 0.001-0.050 wt. %, even more preferably 0.005-0.050 wt. %, even more preferably 0.010-0.050 wt. %, even more preferably 0.015-0.050 wt. %, most preferably 0.015-0.035 wt. %, of the total weight of the composition. This embodiment is, for example, particularly preferred if the animal is a bovine or a horse, more preferably wherein the animal is a calf or a foal, most preferably wherein the animal is a calf.

In a more preferred alternative embodiment, the first sialylated MMO is present at 0.010-1.000 wt. %, preferably 0.050-0.750 wt. %, even more preferably 0.050-0.750 wt. %, even more preferably 0.050-0.500 wt. %, most preferably 0.100-0.500 wt. %, of the total weight of the composition. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In a more preferred alternative embodiment, the first sialylated MMO is present at 0.001-2.500 wt. %, preferably 0.001-1.000 wt. %, more preferably 0.010-1.000 wt. %, even more preferably 0.010-0.500 wt. %, even more preferably 0.015-0.500 wt. %, most preferably 0.015-0.350 wt. %, of the total weight of the composition. This embodiment is, for example, particularly preferred if the animal is a fish or a crustacean, more preferably a fish, even more preferably a fry or a fingerling, even more preferably a fingerling.

In an additional and/or more preferred alternative embodiment, the first sialylated MMO according to the disclosure is provided as a powder in the composition according to the disclosure. Preferably, the powder is as defined earlier in the present section titled “Composition.”

In this context, it is a preferred embodiment, that the second sialylated mammalian milk oligosaccharide (MMO) is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.

In a more preferred embodiment, the second sialylated MMO is present at 0.001-0.250 wt. %, preferably 0.001-0.200 wt. %, more preferably 0.001-0.100 wt. %, even more preferably 0.001-0.075 wt. %, even more preferably 0.001-0.050 wt. %, even more preferably 0.005-0.050 wt. %, even more preferably 0.010-0.050 wt. %, even more preferably 0.010-0.030 wt. %, most preferably 0.010-0.020 wt. %, of the total weight of the composition. This embodiment is, for example, particularly preferred if the animal is a bovine or a horse, more preferably wherein the animal is a calf or a foal, most preferably wherein the animal is a calf.

In a more preferred alternative embodiment, the second sialylated MMO is present at 0.010-1.000 wt. %, preferably 0.025-0.750 wt. %, even more preferably 0.050-0.750 wt. %, even more preferably 0.050-0.500 wt. %, most preferably 0.050-0.250 wt. %, of the total weight of the composition. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In a more preferred alternative embodiment, the second sialylated MMO is present at 0.001-2.500 wt. %, preferably 0.001-1.000 wt. %, more preferably 0.010-1.000 wt. %, even more preferably 0.010-0.750 wt. %, even more preferably 0.010-0.500 wt. %, even more preferably 0.010-0.300 wt. %, most preferably 0.010-0.200 wt. %, of the total weight of the composition. This embodiment is, for example, particularly preferred if the animal is a fish or a crustacean, more preferably a fish, even more preferably a fry or a fingerling, even more preferably a fingerling.

In an additional and/or more preferred alternative embodiment, the second sialylated MMO according to the disclosure is provided as a powder in the composition according to the disclosure. Preferably, the powder is as defined earlier in the present section titled “Composition.”

It is further particularly preferred that the ratio between the amount of the first sialylated MMO in the composition and the amount of the second sialylated MMO in the composition is similar to this ratio as found in the mother's milk of the same species (as described earlier herein). More preferably, the amount of the first sialylated MMO in the composition is higher than the amount of the second sialylated MMO, more preferably the amount of the first sialylated MMO is 1.1 to 40.0, more preferably 1.1 to 35.0, most preferably 1.5 to 35.0, times higher than the amount of the second sialylated oligosaccharide in the composition. Even more preferably, the amount of the first sialylated MMO in the composition is 1.1 to 6.0, more preferably 1.1 to 5.0, even more preferably 1.25-4.0, even more preferably 1.25-3.0, even more preferably 1.5-3.0, most preferably 1.5-2.5, times higher than the amount of the second sialylated oligosaccharide in the composition.

As elaborated in the section titled “Oligosaccharide,” it is preferred that a fucosylated MMO and a sialylated MMO according to the disclosure are administered to the farm animal.

In this context, it is a preferred embodiment that the amount of the sialylated MMO in the composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO in the composition.

It is a more preferred embodiment that the amount of the fucosylated MMO in the composition is lower than the amount of the first sialylated MMO, preferably the amount of the fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of the sialylated MMO in the composition.

As elaborated in the section titled “Oligosaccharide,” it is more preferred that a fucosylated MMO, a first sialylated MMO and a second sialylated MMO (wherein it is preferred that no additional sialylated MMO's are administered) according to the disclosure are administered to the farm animal.

In this context, it a preferred embodiment that the amount of the first sialylated MMO in the composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO in the composition. It is an additional and/or alternative embodiment that the amount of the second sialylated MMO in the composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO in the composition.

It is a more preferred embodiment that the amount of the fucosylated MMO in the composition is lower than the amount of the first sialylated MMO, more preferably the amount of the fucosylated MMO is 1.1 to 10.0, preferably 1.5-7.5, more preferably 2.5-7.5, most preferably 4.0-7.0, times lower than the amount of the first sialylated MMO in the composition.

It is an additional and/or more preferred alternative embodiment that the amount of the fucosylated MMO in the composition is lower than the amount of the second sialylated MMO, more preferably the amount of the fucosylated MMO is 1.1 to 10.0, preferably 1.5-7.5, more preferably 1.5-5.0, most preferably 2.5-5.0, times lower than the amount of the second sialylated MMO in the composition. Preferably, the amount of the fucosylated MMO in the composition is lower than the amount of all sialylated MMOs in the composition, more preferably, the amount of the fucosylated MMO in the composition is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of all sialylated MMOs in the composition.

It is an additional and/or more preferred alternative embodiment that the ratio between the amount of the first sialylated MMO in the composition and the amount of the second sialylated MMO in the composition is similar to this ratio as found in the mother's milk of the same species. More preferably, the amount of the first sialylated MMO in the composition is higher than the amount of the second sialylated MMO, more preferably the amount of the first sialylated MMO is 1.1 to 6.0, preferably 1.1 to 5.0, more preferably 1.25 to 4.0, even more preferably 1.25-3.0, even more preferably 1.5 to 3.0, most preferably 1.5-2.5, times higher than the amount of the second sialylated oligosaccharide in the composition.

The ratio between the fucosylated MMO and the sialylated MMO is preferably as described in the section titled “Method for reducing FCR: general.” The ratio between the fucosylated MMO and the first sialylated MMO in the composition is preferably as described in the section titled “Method for reducing FCR: general.” The ratio between the fucosylated MMO and the second sialylated MMO in the composition is preferably as described in the section titled “Method for reducing FCR: general.”

### Use

In a second aspect, the disclosure provides the use of (i) a fucosylated MMO, (ii) a sialylated MMO, or (iii) a fucosylated MMO and a sialylated MMO to reduce the feed conversion ratio (FCR) of a farm animal.

The FCR is as disclosed in the first aspect of the disclosure.

The farm animal is as disclosed in the section titled “Subject” of the first aspect of the disclosure.

The fucosylated MMO is as disclosed in the sections titled “Oligosaccharide,” “Method for reducing FCR: general” and “Method to reduce FCR: fucosylated MMO” of the first aspect of the disclosure.

The fucosylated MMO is preferably present in a composition referred to in the sections “Composition: general” and “Composition: fucosylated MMO” of the first aspect of the disclosure.

The sialylated MMO is as disclosed in the sections “Oligosaccharide,” “Method for reducing FCR: general” and “Method to reduce FCR: sialylated MMO” of the first aspect of the disclosure.

The sialylated MMO is preferably present in a composition referred to sections “Composition: general” and “Composition: sialylated MMO” of the first aspect of the disclosure.

Each embodiment disclosed in the context of a method to reduce the FCR of a farm animal according to the first aspect of the disclosure, is also disclosed in the context of the second aspect of the disclosure. For example, in analogy with the first aspect of the disclosure, a particularly preferred embodiment of the second aspect of the disclosure is the use of a fucosylated MMO and a sialylated MMO (preferably a first sialylated MMO and a second sialylated MMO, optionally further sialylated MMOs; more preferably a first sialylated MMO and a second sialylated MMO) to reduce the feed conversion ratio (FCR) of a farm animal, wherein the fucosylated MMO and the sialylated MMO are administered to the farm animal in two stages:
stage 1: administering a sialylated MMO (preferably a first sialylated MMO and a second sialylated MMO, optionally further sialylated MMOs; more preferably a first sialylated MMO and a second sialylated MMO) according to the disclosure and optionally administering a fucosylated MMO according to the disclosure; and
stage 2: administering a fucosylated MMO according to the disclosure, preferably without administering a sialylated MMO according to the disclosure;
even more preferably in two stages:
stage 1: administering a sialylated MMO (preferably a first sialylated MMO and a second sialylated MMO, optionally further sialylated MMOs; more preferably a first sialylated MMO and a second sialylated MMO) according to the disclosure and a fucosylated MMO according to the disclosure; and
stage 2: administering a fucosylated MMO according to the disclosure, preferably without administering a sialylated MMO according to the disclosure.

### Method to Increase Body Weight

In a third aspect, the disclosure provides a method to increase the body weight of a farm animal, wherein the method comprises the step of administering to the farm animal a sialylated mammalian milk oligosaccharide (MMO) according to the disclosure and a fucosylated mammalian milk oligosaccharide (MMO) according to the disclosure, preferably wherein the sialylated MMO and the fucosylated MMO are administered to the farm animal in a feeding regimen split in two stages as described in the first aspect of the disclosure.

Preferably, the method increases the average daily gain of a farm animal (i.e., calf fattening when the farm animal is a calf).

The body weight of a farm animal can be readily assessed by the skilled person as disclosed earlier herein.

The increase in body weight and increase of the average daily gain is preferably measured over a period as is disclosed for the reduction of FCR in the first aspect of the disclosure (it is particularly referred to in the section titled “Method to reduce FCR”).

The farm animal is as disclosed in the section titled “Subject” of the first aspect of the disclosure.

The sialylated MMO is as disclosed in the sections “Oligosaccharide,” “Method for reducing FCR: general” and “Method to reduce FCR: sialylated MMO” of the first aspect of the disclosure.

The sialylated MMO is preferably present in a composition referred to in sections “Composition: general” and “Composition: sialylated MMO” of the first aspect of the disclosure.

The fucosylated MMO is as disclosed in the sections “Oligosaccharide,” “Method for reducing FCR: general” and “Method to reduce FCR: fucosylated MMO” of the first aspect of the disclosure.

The fucosylated MMO is preferably present in a composition referred to in sections “Composition: general” and “Composition: fucosylated MMO” of the first aspect of the disclosure.

In a preferred embodiment, the method according to the disclosure increases the body weight (preferably average daily gain) of a farm animal with at least 1.0%, 2.0%, 3.0%, 4.0%, 5.0%, 6.0%, 7.0%, 8.0%, 9.0% or 10.0%, preferably relative to the body weight (preferably average daily gain) of the animal prior to administration of the fucosylated MMO and the sialylated MMO according to the disclosure. Preferably, the body weight (preferably average daily gain) increases with at least 2.0%, more preferably at least 2.5%, even more preferably at least 3.0%, even more preferably at least 3.5%, even more preferably at least 4.0%, even more preferably at least 4.5%, even more preferably at least 5.0%, most preferably at least 7.0%.

In an additional and/or preferred alternative embodiment, the method according to the disclosure increases the body weight (preferably average daily gain) of a farm animal with at least 1.0%, 2.0%, 3.0%, 4.0%, 5.0%, 6.0%, 7.0%, 8.0%, 9.0% or 10.0%, preferably relative to the body weight (preferably average daily gain) of the animal prior to administration of the fucosylated MMO and the sialylated MMO according to the disclosure. Preferably, the body weight (preferably average daily gain) increase is at least 2.0%, more preferably at least 2.5%, even more preferably at least 3.0%, even more preferably at least 3.5%, even more preferably at least 4.0%, even more preferably at least 4.5%, even more preferably at least 5.0%, most preferably at least 7.0%.

### Specific Embodiments

The disclosure preferably relates to the following specific embodiments:
1. A method to reduce the feed conversion ratio (FCR) of a farm animal, wherein the method comprises the step of administering to a farm animal: (i) a fucosylated mammalian milk oligosaccharide (MMO), (ii) a sialylated MMO, or (iii) a fucosylated MMO and a sialylated MMO.
2. A method according to embodiment 1, wherein the animal is a non-adult animal.
3. A method according to embodiment 1 or 2, wherein the animal is multigastric animal, preferably a ruminant.
4. A method according to embodiment 1 or 2, wherein the farm animal is selected from the group consisting of bovine, pig, sheep, goat, horse, donkey, mule, hinny, camelid (preferably llama or alpaca), rabbit, fish, crustacean and poultry; preferably selected from the group consisting of bovine, pig, sheep, goat, horse, camelid (preferably llama or alpaca), rabbit, fish, crustacean and poultry; more preferably selected from the group consisting of bovine, pig, sheep, goat, horse, camelid (preferably llama or alpaca), rabbit, fish and poultry; even more preferably selected from the group consisting of bovine, pig, sheep, goat, horse, camelid (preferably llama or alpaca) and poultry; even more preferably selected from the group consisting of bovine, pig, sheep, goat, camelid (preferably llama or alpaca) and poultry; even more preferably selected from the group consisting of bovine, pig, sheep, goat, and poultry; even more preferably selected from the group consisting of bovine, pig, sheep and goat; even more preferably selected from the group consisting of bovine, sheep and goat; most preferably the farm animal is a bovine.
5. A method according to any one of embodiments 1 to 4, wherein the animal is a bovine, preferably a calf.
6. A method according to any one of embodiments 1 to 5, wherein the fucosylated MMO consists of 3-8, preferably 3-7, more preferably 3-6, even more preferably 3-5, monosaccharides.
7. A method according to any one of embodiments 1 to 6, wherein the fucosylated MMO is a neutral oligosaccharide.
8. A method according to any one of embodiments 1 to 7, wherein the fucosylated MMO comprises a fucose that is linked to a monosaccharide in an α-1,2-, α-1,3- or α-1,4-linkage, preferably an α-1,2- or an α-1,3-linkage, more preferably an α-1,2-linkage, and wherein the monosaccharide is preferably selected from glucose, N-acetylglucosamine and galactose, more preferably the monosaccharide is glucose or galactose, even more preferably the monosaccharide is galactose.
9. A method according to any one of embodiments 1 to 8, wherein the fucosylated MMO comprises a lactose, a lacto-N-biose (LNB) or N-acetyllactosamine (LacNAc) at its reducing end, preferably the fucosylated MMO comprises lactose or LacNAc at its reducing end, more preferably the fucosylated MMO comprises lactose at its reducing end.
10. A method according to any one of embodiments 1 to 9, wherein the fucosylated MMO is selected from the group consisting of 2′-fucosyllactose (2′FL), 3-fucosyllactose (3-FL), difucosyllactose (diFL), 2′-fucosyl-N-acetyllactosamine (2′FlacNAc), difucosyl-N-acetyllactosamine (diFLacNAc), 3-fucosyl-N-acetyllactosamine (3FlacNAc), 2′-fucosyllacto-N-biose (2′FLNB), 4-fucosyllacto-N-biose (4FLNB), difucosyllacto-N-biose (diFLNB), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH).
11. A method according to any one of embodiments 1 to 10, wherein the fucosylated oligosaccharide is selected from the group consisting of 2′FL, diFL, LNFP I, GalNAc-LNFP I, Gal-LNFP I, LNDFH I, lewis b-lewis x, DFLNH (a), TFLNH, LNnFP I, 2′FLNB, diFLNB, 2′FlacNAc and diFLacNAc, preferably selected from a group consisting of 2′FL, diFL, LNFP I, LNnFP I, 2′FLNB, diFLNB, 2′FlacNAc and diFLacNAc, more preferably selected from a group consisting of 2′FL, diFL, 2′FLNB, diFLNB, 2′FlacNAc and diFLacNAc, even more preferably selected from a group consisting of 2′FL, 2′FLNB and 2′FlacNAc, even more preferably wherein the fucosylated MMO is 2′FL or 2′FLNB, most preferably wherein the fucosylated MMO is 2′FL.
12. A method according to any one of embodiments 1 to 11, wherein the fucosylated MMO is 2′FL.
13. A method according to any one of embodiments 1 to 12, wherein the fucosylated MMO is linked to a carrier for delivery of the fucosylated MMO, preferably wherein the carrier is a ceramide-based carrier or a polypeptide-based carrier, more preferably wherein the carrier is a ceramide-based carrier.
14. A method according to any one of embodiments 1 to 13, wherein the fucosylated MMO is encapsulated.
15. A method according to any one of embodiments 1 to 14, wherein an effective amount of the fucosylated MMO is administered to the farm animal.
16. A method according to any one of embodiments 1 to 15, wherein the fucosylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g.
17. A method according to any one of embodiments 1 to 15, wherein the fucosylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.
18. A method according to any one of embodiments 1 to 15, wherein the fucosylated MMO is administered at a daily dose of 0.01-1.0 g, preferably 0.01-0.75 g, more preferably 0.01-0.65 g, even more preferably 0.01-0.60 g, even more preferably 0.01-0.50 g, even more preferably 0.015-0.50 g, even more preferably 0.015-0.40 g, even more preferably 0.015-0.30 g, even more preferably 0.015-0.20 g, most preferably 0.015-0.10 g, preferably wherein the animal is a bovine or a horse, more preferably wherein the animal is a calf or a foal, even more preferably wherein the animal is a calf.
19. A method according to any one of embodiments 1 to 15, wherein the fucosylated MMO is administered at a daily dose of 0.01-5.0 g, preferably 0.1-2.5 g, more preferably 0.1-1.5 g, even more preferably 0.1-1.2 g, even more preferably 0.2-1.2 g, even more preferably 0.3-1.2 g, even more preferably 0.4-1.2 g, most preferably 0.5-1.2 g, preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
20. A method according to any one of embodiments 1 to 15, wherein the fucosylated MMO is administered at a daily dose of 0.0001-1.0 g, preferably 0.0001-0.50 g, more preferably 0.0001-0.10 g, even more preferably 0.0001-0.075 g, even more preferably 0.0001-0.050 g, even more preferably 0.0002-0.050 g, even more preferably 0.0005-0.050 g, most preferably 0.0005-0.025 g, preferably wherein the animal is a fish or a crustacean, more preferably a fish, even more preferably a fry or a fingerling, even more preferably a fingerling.
21. A method according to any one of embodiments 1 to 20, wherein the fucosylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of the animal.
22. A method according to any one of embodiments 1 to 20, wherein the fucosylated MMO is administered at a daily dose of 0.1-50.0 mg, preferably 0.1-30.0 mg, more preferably 0.1-25.0 mg, even more preferably 0.1-20.0 mg, even more preferably 0.1-15.0 mg, even more preferably 0.1-10.0 mg, even more preferably 0.25-10.0 mg, most preferably 0.5-10.0 mg, per kg bodyweight of the animal, preferably wherein the animal is a bovine or a horse, more preferably wherein the animal is a calf or a foal, even more preferably wherein the animal is a calf.
23. A method according to any one of embodiments 1 to 20, wherein the fucosylated MMO is administered at a daily dose of 0.1-150.0 mg, preferably 0.1-125.0 mg, more preferably 1.0-125.0 mg, even more preferably 5.0-125.0 mg, even more preferably 5.0-100.0 mg, even more preferably 5.0-80.0 mg, most preferably 10.0-80.0 mg, per kg bodyweight of the animal, preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
24. A method according to any one of embodiments 1 to 20, wherein the fucosylated MMO is administered at a daily dose of 0.1-70.0 mg, preferably 0.1-60.0 mg, more preferably 0.1-50.0 mg, even more preferably 0.5-50.0 mg, even more preferably 1.0-50.0 mg, even more preferably 2.5-50.0 mg, even more preferably 5.0-50.0 mg, even more preferably 10.0-50.0 mg, most preferably 10.0-40.0 mg, per kg bodyweight of the animal, preferably wherein the animal is a fish or a crustacean, more preferably a fish, even more preferably a fry or a fingerling, even more preferably a fingerling.
25. A method according to any one of embodiments 1 to 24, wherein the fucosylated MMO is administered to the animal at least once, preferably at least twice, between birth (0 weeks) and 5 weeks of age, preferably between birth and 4 weeks of age, more preferably between birth and 3 weeks of age.
26. A method according to any one of embodiments 1 to 25, wherein the fucosylated MMO is administered to the animal for at least 1 week, preferably at least 2 weeks, more preferably at least 3 weeks, even more preferably at least 4 weeks, most preferably at least 6 weeks.
27. A method according to any one of embodiments 1 to 26, wherein the fucosylated MMO is administered to the animal for ≤32 weeks, preferably ≤28 weeks, more preferably ≤24 weeks, even more preferably ≤20 weeks, even more preferably ≤16 weeks, most preferably ≤12 weeks.
28. A method according to any one of embodiments 1 to 27, wherein the fucosylated MMO is administered to the animal for 1-30, preferably 1-25, more preferably 1-20, even more preferably 1-15, even more preferably 1-10, even more preferably 1-7.5, even more preferably 1-5, even more preferably 1-4, most preferably 1-3, consecutive weeks.
29. A method according to any one of embodiments 1 to 28, wherein the fucosylated MMO is administered to the animal for:
   if subject is bovine: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
   if subject is a pig: 1-18, preferably 1-16, more preferably 1-14, even more preferably 1-12, even more preferably 2-12, even more preferably 3-12, even more preferably 4-12, most preferably 6-12, consecutive weeks;
   if subject is a sheep: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
   if subject is a goat: 1-18, preferably 1-16, more preferably 1-14, even more preferably 1-12, even more preferably 2-12, even more preferably 3-12, even more preferably 4-12, most preferably 6-12, consecutive weeks;
   if subject is a horse: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
   if subject is a camelid: 1-14, preferably 1-12, more preferably 2-12, even more preferably 3-12, even more preferably 4-12, most preferably 6-12, consecutive weeks;
   if subject is a rabbit: 1-12, preferably 2-12, more preferably 3-12, even more preferably 4-12, most preferably 6-12, consecutive weeks;
   if subject is a fish or a crustacean: 1-36, preferably 1-32, more preferably 1-24, even more preferably 1-18, even more preferably 1-12, even more preferably 2-12, even more preferably 3-12, even more preferably 6-12, consecutive months;
   if subject is poultry (but not a turkey): 1-8, preferably 1-7, more preferably 2-7, even more preferably 3-7, most preferably 4-7, consecutive weeks;
   if subject is a turkey: 1-18, preferably 1-16, more preferably 1-14, even more preferably 1-12, even more preferably 2-12, even more preferably 3-12, even more preferably 4-12, most preferably 6-12, consecutive weeks.
30. A method according to any one of embodiments 1 to 29, wherein the fucosylated MMO is part of a composition.
31. A method according to embodiment 30, wherein the fucosylated MMO is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.
32. A method according to embodiment 30 or 31, wherein the fucosylated MMO is present at 0.001-0.250 wt. %, preferably 0.001-0.200 wt. %, more preferably 0.001-0.100 wt. %, even more preferably 0.001-0.020 wt. %, even more preferably 0.001-0.010 wt. %, even more preferably 0.002-0.010 wt. %, even more preferably 0.003-0.010 wt. %, even more preferably 0.003-0.0075 wt. %, most preferably 0.003-0.006 wt. %, of the total weight of the composition, preferably wherein the animal is a bovine or a horse, more preferably wherein the animal is a calf or a foal, even more preferably wherein the animal is a calf.
33. A method according to embodiment 30 or 31, wherein the fucosylated MMO is present at 0.010-1.000 wt. %, preferably 0.010-0.750 wt. %, more preferably 0.010-0.500 wt. %, even more preferably 0.010-0.400 wt. %, even more preferably 0.050-0.400 wt. %, even more preferably 0.100-0.400 wt. %, most preferably 0.100-0.300 wt. %, of the total weight of the composition, preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
34. A method according to embodiment 30 or 31, wherein the fucosylated MMO is present at 0.001-2.500 wt. %, preferably 0.001-1.000 wt. %, more preferably 0.010-1.000 wt. %, even more preferably 0.025-1.000 wt. %, even more preferably 0.025-0.750 wt. %, even more preferably 0.025-0.500 wt. %, even more preferably 0.025-0.250 wt. %, even more preferably 0.025-0.100 wt. %, most preferably 0.025-0.075 wt. %, of the total weight of the composition, preferably wherein the animal is a fish or a crustacean, more preferably a fish, even more preferably a fry or a fingerling, even more preferably a fingerling.
35. A method according to any one of embodiments 1 to 34, wherein the sialylated MMO consists of 3-8, preferably 3-7, more preferably 3-6, even more preferably 3-5, monosaccharides.
36. A method according to any one of embodiments 1 to 35, wherein the sialylated MMO comprises a sialic acid, preferably a Neu5Ac, that is linked to a monosaccharide in an α-2,3-, α-2,6- or α-2,8-linkage, preferably an α-2,3- or an α-2,6-linkage, and wherein the monosaccharide is preferably selected from galactose, N-acetylglucosamine and Neu5Ac, more preferably the monosaccharide is galactose or N-acetylglucosamine, even more preferably the monosaccharide is galactose.
37. A method according to any one of embodiments 1 to 36, wherein the sialylated MMO comprises a lactose, a lacto-N-biose (LNB) or N-acetyllactosamine (LacNAc) at its reducing end, lactose or LacNAc at is reducing end, most preferably the sialylated MMO comprises lactose at its reducing end.
38. A method according to any one of embodiments 1 to 37, wherein the sialylated MMO is selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SlacNAc), 6′-sialyllactosamine (6′SlacNAc) and sialyl Lewis x; preferably the sialylated MMO is selected from the group consisting of 3′SL, 6′SL, 3′S-2′FL, 6′S-2′FL, 3′S-3-FL, 6′S-3-FL, LST a, LST b, LST c, LST d, 3′SLNB, 6′SLNB, sialyl Lewis a, 3′SlacNAc, 6′SlacNAc and sialyl Lewis x; more preferably the sialylated MMO is selected from the group consisting of 3′SL, 6′SL, LST a, LST b, LST c, LST d, 3′SLNB, 6′SLNB, sialyl Lewis a, 3′SlacNAc, 6′SlacNAc and sialyl Lewis x; even more preferably the sialylated MMO is selected from the group consisting of 3′SL, 6′SL, 3′SLNB, 6′SLNB, 3′SlacNAc and 6′SlacNAc; even more preferably the sialylated MMO is 3′-sialyllactose (3′SL) or 6′-sialyllactose (6′SL), most preferably the sialylated MMO is 3′SL.
39. A method according to any one of embodiments 1 to 38, wherein the sialylated MMO comprises a sialic acid, preferably a Neu5Ac, that is linked to a monosaccharide in an α-2,3-linkage, wherein the monosaccharide is preferably selected from galactose, N-acetylglucosamine and Neu5Ac, more preferably the monosaccharide is galactose or N-acetylglucosamine.
40. A method according to any one of embodiments 1 to 39, wherein the sialylated MMO is selected from the group consisting of 3′-sialyllactose (3′SL), 3′-sialyl-2′-fucosyllactose (3′S-2′FL), 3′-sialyl-3′-fucosyllactose (3′S-3′FL), LST a, LST d, 3′-sialyl-N-acetyllactosamine (3′SlacNAc), 3′-sialyl-3-fucosyllactose and 3′-sialyllacto-N-biose (3′SLNB), preferably selected from the group consisting of 3′-sialyllactose (3′SL), LST a, LST d, 3′-sialyl-N-acetyllactosamine (3′SlacNAc) and 3′-sialyllacto-N-biose (3′SLNB), more preferably selected from the group consisting of 3′-sialyllactose (3′SL), 3′-sialyl-N-acetyllactosamine (3′SlacNAc) and 3′-sialyllacto-N-biose (3′SLNB), most preferably wherein the sialylated MMO is 3′-sialyllactose (3′SL).
41. A method according to any one of embodiments 1 to 40, wherein the sialylated MMO is 3′-sialyllactose (3′SL).
42. A method according to any one of embodiments 1 to 38, wherein the sialylated MMO comprises a sialic acid, preferably a Neu5Ac, that is linked to a monosaccharide in an α-2,6-linkage, wherein the monosaccharide is preferably selected from galactose, N-acetylglucosamine and Neu5Ac, more preferably the monosaccharide is galactose or N-acetylglucosamine.
43. A method according to any one of embodiments 1 to 38 and 42, wherein the sialylated MMO is selected from the group consisting of 6′-sialyllactose (6′SL), 6′-sialyl-2′-fucosyllactose (6′S-2′FL), 6′-sialyl-3′-fucosyllactose (6′S-3′FL), Neu5Ac-α2,6-[GlcNAc-b1,3]Gal-b1,4-Glc, LST b, LST c, 6′-sialyl-N-acetyllactosamine (6′SlacNAc) and 6′-sialyllacto-N-biose (6′SLNB), preferably selected from the group consisting of 6′-sialyllactose (6′SL), LST b, LST c, 6′-sialyl-N-acetyllactosamine (6′SlacNAc) and 6′-sialyllacto-N-biose (6′SLNB), more preferably selected from the group consisting of 6′-sialyllactose (6′SL), 6′-sialyl-N-acetyllactosamine (6′SlacNAc) and 6′-sialyllacto-N-biose (6′SLNB), most preferably wherein the sialylated MMO is 6′-sialyllactose (6′SL).
44. A method according to any one of embodiments 1 to 38, 42 and 43, wherein the sialylated MMO is 6′-sialyllactose (6′SL).
45. A method according to any one of embodiments 1 to 44, wherein the sialylated MMO is linked to a carrier for delivery of the sialylated MMO, preferably wherein the carrier is a ceramide-based carrier or a polypeptide-based carrier, more preferably wherein the carrier is a ceramide-based carrier.
46. A method according to any one of embodiments 1 to 45, wherein the sialylated MMO is encapsulated.
47. A method according to any one of embodiments 1 to 446 wherein an effective amount of the sialylated MMO is administered to the farm animal.
48. A method according to any one of embodiments 1 to 47, wherein the sialylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g.
49. A method according to any one of embodiments 1 to 47, wherein the sialylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.
50. A method according to any one of embodiments 1 to 47, wherein the sialylated MMO is administered at a daily dose of 0.01-1.0 g, preferably 0.01-0.75 g, more preferably 0.025-0.75 g, even more preferably 0.05-0.75 g, most preferably 0.05-0.50 g, preferably wherein the animal is a bovine or a horse, more preferably wherein the animal is a calf or a foal, even more preferably wherein the animal is a calf.
51. A method according to any one of embodiments 1 to 47, wherein the sialylated MMO is administered at a daily dose of 0.01-5.0 g, preferably 0.1-2.5 g, more preferably 0.1-2.0 g, even more preferably 0.1-1.5 g, even more preferably 0.2-1.5 g, even more preferably 0.3-1.5 g, even more preferably 0.4-1.5 g, most preferably 0.5-1.5 g, preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
52. A method according to any one of embodiments 1 to 47, wherein the sialylated MMO is administered at a daily dose of 0.0001-1.0 g, preferably 0.0001-0.50 g, more preferably 0.0001-0.10 g, even more preferably 0.0001-0.075 g, even more preferably 0.0001-0.050 g, even more preferably 0.0002-0.050 g, even more preferably 0.0005-0.050 g, most preferably 0.0005-0.025 g, preferably wherein the animal is a fish or a crustacean, more preferably a fish, even more preferably a fry or a fingerling, even more preferably a fingerling.
53. A method according to any one of embodiments 1 to 52, wherein the sialylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of the animal.
54. A method according to any one of embodiments 1 to 52, wherein the sialylated MMO is administered at a daily dose of 0.1-100.0 mg, preferably 0.1-75.0 mg, more preferably 0.5-75.0 mg, even more preferably 1.0-75.0 mg, even more preferably 2.5-75.0 mg, most preferably 2.5-50.0 mg, per kg bodyweight of the animal, preferably wherein the animal is a bovine or a horse, more preferably wherein the animal is a calf or a foal, even more preferably wherein the animal is a calf.
55. A method according to any one of embodiments 1 to 52, wherein the sialylated MMO is administered at a daily dose of 0.1-150.0 mg, preferably 0.5-150.0 mg, more preferably 1.0-150.0 mg, even more preferably 1.0-125.0 mg, even more preferably 2.5-125.0 mg, even more preferably 5.0-125.0 mg, even more preferably 10.0-125.0 mg, even more preferably 25.0-125.0 mg, even more preferably 50.0-125.0 mg, most preferably 75.0-125.0 mg, per kg bodyweight of the animal, preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
56. A method according to any one of embodiments 1 to 52, wherein the sialylated MMO is administered at a daily dose of 0.1-70.0 mg, preferably 0.1-60.0 mg, more preferably 0.1-50.0 mg, even more preferably 0.5-50.0 mg, even more preferably 1.0-50.0 mg, even more preferably 2.5-50.0 mg, even more preferably 5.0-50.0 mg, even more preferably 10.0-50.0 mg, most preferably 10.0-40.0 mg, per kg bodyweight of the animal, preferably wherein the animal is a fish or a crustacean, more preferably a fish, even more preferably a fry or a fingerling, even more preferably a fingerling.
57. A method according to any one of embodiments 1 to 56, wherein the sialylated MMO is administered to the animal at least once, preferably at least twice, between birth (0 weeks) and 5 weeks of age, preferably between birth and 4 weeks of age, more preferably between birth and 3 weeks of age.
58. A method according to any one of embodiments 1 to 57, wherein the sialylated MMO is administered to the animal for at least 1 week, preferably at least 2 weeks, more preferably at least 3 weeks, even more preferably at least 4 weeks, most preferably at least 6 weeks.
59. A method according to any one of embodiments 1 to 58, wherein the sialylated MMO is administered to the animal for ≤32 weeks, preferably ≤28 weeks, more preferably ≤24 weeks, even more preferably ≤20 weeks, even more preferably ≤16 weeks, most preferably ≤12 weeks.
60. A method according to any one of embodiments 1 to 59, wherein the sialylated MMO is administered to the animal for 1-30, preferably 1-25, more preferably 1-20, even more preferably 1-15, even more preferably 1-10, even more preferably 1-7.5, even more preferably 1-5, even more preferably 1-4, most preferably 1-3, consecutive weeks.
61. A method according to any one of embodiments 1 to 60, wherein the sialylated MMO is administered to the animal for:
   if subject is bovine: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
   if subject is a pig: 1-18, preferably 1-16, more preferably 1-14, even more preferably 1-12, even more preferably 2-12, even more preferably 3-12, even more preferably 4-12, most preferably 6-12, consecutive weeks;
   if subject is a sheep: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
   if subject is a goat: 1-18, preferably 1-16, more preferably 1-14, even more preferably 1-12, even more preferably 2-12, even more preferably 3-12, even more preferably 4-12, most preferably 6-12, consecutive weeks;
   if subject is a horse: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
   if subject is a camelid: 1-14, preferably 1-12, more preferably 2-12, even more preferably 3-12, even more preferably 4-12, most preferably 6-12, consecutive weeks;
   if subject is a rabbit: 1-12, preferably 2-12, more preferably 3-12, even more preferably 4-12, most preferably 6-12, consecutive weeks;
   if subject is a fish or a crustacean: 1-36, preferably 1-32, more preferably 1-24, even more preferably 1-18, even more preferably 1-12, even more preferably 2-12, even more preferably 3-12, even more preferably 6-12, consecutive months;
   if subject is poultry (but not a turkey): 1-8, preferably 1-7, more preferably 2-7, even more preferably 3-7, most preferably 4-7, consecutive weeks;
   if subject is a turkey: 1-18, preferably 1-16, more preferably 1-14, even more preferably 1-12, even more preferably 2-12, even more preferably 3-12, even more preferably 4-12, most preferably 6-12, consecutive weeks.
62. A method according to any one of embodiments 1 to 61, wherein the sialylated MMO is part of a composition.
63. A method according to embodiment 62, wherein the sialylated MMO is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.
64. A method according to embodiment 62 or 63, wherein the sialylated MMO is present at 0.001-0.250 wt. %, preferably 0.001-0.200 wt. %, more preferably 0.001-0.100 wt. %, even more preferably 0.005-0.100 wt. %, even more preferably 0.005-0.075 wt. %, even more preferably 0.005-0.050 wt. %, even more preferably 0.005-0.040 wt. %, most preferably 0.010-0.040 wt. %, of the total weight of the composition, preferably wherein the animal is a bovine or a horse, more preferably wherein the animal is a calf or a foal, even more preferably wherein the animal is a calf.
65. A method according to embodiment 62 or 63, wherein the sialylated MMO is present at 0.010-1.000 wt. %, preferably 0.010-0.750 wt. %, even more preferably 0.010-0.750 wt. %, even more preferably 0.010-0.500 wt. %, even more preferably 0.025-0.500 wt. %, most preferably 0.050-0.500 wt. %, of the total weight of the composition, preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
66. A method according to embodiment 62 or 63, wherein the sialylated MMO is present at 0.001-2.500 wt. %, preferably 0.001-1.000 wt. %, more preferably 0.010-1.000 wt. %, even more preferably 0.025-1.000 wt. %, even more preferably 0.025-0.750 wt. %, even more preferably 0.025-0.500 wt. %, even more preferably 0.025-0.500 wt. %, even more preferably 0.025-0.400 wt. %, most preferably 0.050-0.400 wt. %, of the total weight of the composition, preferably wherein the animal is a fish or a crustacean, more preferably a fish, even more preferably a fry or a fingerling, even more preferably a fingerling.
67. A method according to embodiment 62 or 63, wherein the sialylated MMO is present at 0.001-0.250 wt. %, preferably 0.001-0.200 wt. %, more preferably 0.001-0.100 wt. %, even more preferably 0.001-0.050 wt. %, even more preferably 0.005-0.050 wt. %, even more preferably 0.010-0.050 wt. %, even more preferably 0.015-0.050 wt. %, most preferably 0.015-0.035 wt. %, of the total weight of the composition, preferably wherein the animal is a bovine or a horse, more preferably wherein the animal is a calf or a foal, even more preferably wherein the animal is a calf.
68. A method according to embodiment 62 or 63, wherein the sialylated MMO is present at 0.010-1.000 wt. %, preferably 0.050-0.750 wt. %, even more preferably 0.050-0.750 wt. %, even more preferably 0.050-0.500 wt. %, most preferably 0.100-0.500 wt. %, of the total weight of the composition, preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
69. A method according to embodiment 62 or 63, wherein the sialylated MMO is present at 0.001-2.500 wt. %, preferably 0.001-1.000 wt. %, more preferably 0.010-1.000 wt. %, even more preferably 0.010-0.500 wt. %, even more preferably 0.015-0.500 wt. %, most preferably 0.015-0.350 wt. %, of the total weight of the composition, preferably wherein the animal is a fish or a crustacean, more preferably a fish, even more preferably a fry or a fingerling, even more preferably a fingerling.
70. A method according to any one of embodiments 1 to 69, wherein the fucosylated MMO and the sialylated MMO are administered to the animal and wherein the amount of the sialylated MMO administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal.
71. A method according to any one of embodiments 1 to 69, wherein the fucosylated MMO and the sialylated MMO are administered to the animal and wherein the amount of the fucosylated MMO that is administered to the animal is lower, preferably 1.1 to 15.0 times lower, more preferably 1.5 to 15.0 times lower, even more preferably 2.5 to 15.0 times lower, even more preferably 5.0 to 15.0 times lower, even more preferably 5.0 to 12.5 times lower, even more preferably 5.0 to 10.0 times lower, than the amount of the sialylated MMO administered to the animal.
72. A method according to any one of embodiments 62 to 69, wherein the amount of the sialylated MMO in the composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO in the composition.
73. A method according to any one of embodiments 62 to 69, wherein the amount of the fucosylated MMO in the composition is lower, preferably 1.1 to 15.0 times lower, more preferably 1.5 to 15.0 times lower, even more preferably 2.5 to 15.0 times lower, even more preferably 5.0 to 15.0 times lower, even more preferably 5.0 to 12.5 times lower, even more preferably 5.0 to 10.0 times lower, than the amount of the sialylated MMO in the composition.
74. A method according to any one of embodiments 1 to 73, wherein the sialylated MMO is administered to the animal from the start of the method.
75. A method according to any one of embodiments 1 to 74, wherein the fucosylated MMO and the sialylated MMO are administered to the animal and wherein the fucosylated MMO is administered to the animal after at least 0%, preferably at least 10%, more preferably at least 25%, even more preferably at least 30%, most preferably at least 50%, of the total period wherein MMOs are administered to the farm animal has passed.
76. A method according to any one of embodiments 1 to 75, wherein administering a sialylated MMO further comprises administering a second sialylated MMO.
77. A method according to embodiment 76, wherein the second sialylated MMO consists of 3-8, preferably 3-7, more preferably 3-6, even more preferably 3-5, monosaccharides.
78. A method according to embodiment 76 or 77, wherein the second sialylated MMO comprises a sialic acid, preferably a Neu5Ac, that is linked to a monosaccharide in an α-2,3-, α-2,6- or α-2,8-linkage, preferably an α-2,3- or an α-2,6-linkage, more preferably an α-2,6-linkage, and wherein the monosaccharide is preferably selected from galactose, N-acetylglucosamine and Neu5Ac, more preferably the monosaccharide is galactose or N-acetylglucosamine, even more preferably the monosaccharide is galactose.
79. A method according to any one of embodiments 76 to 78, wherein the second sialylated MMO comprises a lactose, a lacto-N-biose (LNB) or N-acetyllactosamine (LacNAc) at its reducing end, lactose or LacNAc at is reducing end, most preferably the second sialylated MMO comprises lactose at its reducing end.
80. A method according to any one of embodiments 76 to 79, wherein the second sialylated MMO is selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SlacNAc), 6′-sialyllactosamine (6′SlacNAc) and sialyl Lewis x; preferably the sialylated MMO is selected from the group consisting of 3′SL, 6′SL, 3′S-2′FL, 6′S-2′FL, 3′S-3-FL, 6′S-3-FL, LST a, LST b, LST c, LST d, 3′SLNB, 6′SLNB, sialyl Lewis a, 3′SlacNAc, 6′SlacNAc and sialyl Lewis x; more preferably the sialylated MMO is selected from the group consisting of 3′SL, 6′SL, LST a, LST b, LST c, LST d, 3′SLNB, 6′SLNB, sialyl Lewis a, 3′SlacNAc, 6′SlacNAc and sialyl Lewis x; even more preferably the sialylated MMO is selected from the group consisting of 3′SL, 6′SL, 3′SLNB, 6′SLNB, 3′SlacNAc and 6′SlacNAc; even more preferably the sialylated MMO is 3′-sialyllactose (3′SL) or 6′-sialyllactose (6′SL), most preferably the sialylated MMO is 6′SL.
81. A method according to any one of embodiments 76 to 80, wherein the second sialylated MMO comprises a sialic acid, preferably a Neu5Ac, that is linked to a monosaccharide in an α-2,6-linkage, wherein the monosaccharide is preferably selected from galactose, N-acetylglucosamine and Neu5Ac, more preferably the monosaccharide is galactose or N-acetylglucosamine.
82. A method according to any one of embodiments 76 to 81, wherein the second sialylated MMO is selected from the group consisting of 6′-sialyllactose (6′SL), 6′-sialyl-2′-fucosyllactose (6′S-2′FL), 6′-sialyl-3′-fucosyllactose (6′S-3′FL), Neu5Ac-α2,6-[GlcNAc-b1,3]Gal-b1,4-Glc, LST b, LST c, 6′-sialyl-N-acetyllactosamine (6′SlacNAc) and 6′-sialyllacto-N-biose (6′SLNB), preferably selected from the group consisting of 6′-sialyllactose (6′SL), LST b, LST c, 6′-sialyl-N-acetyllactosamine (6′SlacNAc) and 6′-sialyllacto-N-biose (6′SLNB), more preferably selected from the group consisting of 6′-sialyllactose (6′SL), 6′-sialyl-N-acetyllactosamine (6′SlacNAc) and 6′-sialyllacto-N-biose (6′SLNB), most preferably wherein the second sialylated MMO is 6′-sialyllactose (6′SL).
83. A method according to any one of embodiments 76 to 82, wherein the second sialylated MMO is 6′-sialyllactose (6′SL).
84. A method according to any one of embodiments 76 to 83, wherein the second sialylated MMO is linked to a carrier for delivery of the second sialylated MMO, preferably wherein the carrier is a ceramide-based carrier or a polypeptide-based carrier, more preferably wherein the carrier is a ceramide-based carrier.
85. A method according to any one of embodiments 76 to 84, wherein the second sialylated MMO is encapsulated.
86. A method according to any one of embodiments 76 to 85, wherein an effective amount of the second sialylated MMO is administered to the farm animal.
87. A method according to any one of embodiments 76 to 86, wherein the second sialylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g.
88. A method according to any one of embodiments 76 to 86, wherein the second sialylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.
89. A method according to any one of embodiments 76 to 86, wherein the second sialylated MMO is administered at a daily dose of 0.01-1.0 g, preferably 0.01-0.75 g, more preferably 0.025-0.75 g, even more preferably 0.025-0.50 g, most preferably 0.025-0.25 g, preferably wherein the animal is a bovine or a horse, more preferably wherein the animal is a calf or a foal, even more preferably wherein the animal is a calf.
90. A method according to any one of embodiments 76 to 86, wherein the second sialylated MMO is administered at a daily dose of 0.01-5.0 g, preferably 0.1-2.5 g, more preferably 0.1-2.0 g, even more preferably 0.1-1.5 g, even more preferably 0.1-1.2 g, even more preferably 0.15-1.2 g, even more preferably 0.2-1.2 g, most preferably 0.25-1.2 g, preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
91. A method according to any one of embodiments 76 to 86, wherein the second sialylated MMO is administered at a daily dose of 0.0001-1.0 g, preferably 0.0001-0.50 g, more preferably 0.0001-0.10 g, even more preferably 0.0001-0.075 g, even more preferably 0.0001-0.050 g, even more preferably 0.0002-0.050 g, even more preferably 0.0002-0.030 g, most preferably 0.0002-0.020 g, preferably wherein the animal is a fish or a crustacean, more preferably a fish, even more preferably a fry or a fingerling, even more preferably a fingerling.
92. A method according to any one of embodiments 76 to 91, wherein the second sialylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of the animal.
93. A method according to any one of embodiments 76 to 91, wherein the second sialylated MMO is administered at a daily dose of 0.1-100.0 mg, preferably 0.1-75.0 mg, more preferably 0.25-75.0 mg, even more preferably 0.5-75.0 mg, even more preferably 1.0-75.0 mg, even more preferably 1.0-50.0 mg, most preferably 1.0-25.0 mg, per kg bodyweight of the animal, preferably wherein the animal is a bovine or a horse, more preferably wherein the animal is a calf or a foal, even more preferably wherein the animal is a calf.
94. A method according to any one of embodiments 76 to 91, wherein the sialylated MMO is administered at a daily dose of 0.1-150.0 mg, preferably 1.0-150.0 mg, more preferably 1.0-125.0 mg, even more preferably 2.5-125.0 mg, even more preferably 5.0-125.0 mg, even more preferably 10.0-125.0 mg, even more preferably 25.0-125.0 mg, even more preferably 25.0-100.0 mg, most preferably 25.0-80.0, per kg bodyweight of the animal, preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
95. A method according to any one of embodiments 76 to 91, wherein the sialylated MMO is administered at a daily dose of 0.1-70.0 mg, preferably 0.1-60.0 mg, more preferably 0.1-50.0 mg, even more preferably 0.5-50.0 mg, even more preferably 1.0-50.0 mg, even more preferably 2.5-50.0 mg, even more preferably 5.0-50.0 mg, even more preferably 5.0-40.0 mg, most preferably 5.0-30.0 mg, per kg bodyweight of the animal, preferably wherein the animal is a fish or a crustacean, more preferably a fish, even more preferably a fry or a fingerling, even more preferably a fingerling.
96. A method according to any one of embodiments 76 to 95, wherein the second sialylated MMO is administered to the animal at least once, preferably at least twice, between birth (0 weeks) and 5 weeks of age, preferably between birth and 4 weeks of age, more preferably between birth and 3 weeks of age.
97. A method according to any one of embodiments 76 to 96, wherein the second sialylated MMO is administered to the animal for at least 1 week, preferably at least 2 weeks, more preferably at least 3 weeks, even more preferably at least 4 weeks, most preferably at least 6 weeks.
98. A method according to any one of embodiments 76 to 97, wherein the second sialylated MMO is administered to the animal for ≤32 weeks, preferably ≤28 weeks, more preferably ≤24 weeks, even more preferably ≤20 weeks, even more preferably ≤16 weeks, most preferably ≤12 weeks.
99. A method according to any one of embodiments 76 to 98, wherein the second sialylated MMO is administered to the animal for 1-30, preferably 1-25, more preferably 1-20, even more preferably 1-15, even more preferably 1-10, even more preferably 1-7.5, even more preferably 1-5, even more preferably 1-4, most preferably 1-3, consecutive weeks.
100. A method according to any one of embodiments 76 to 99, wherein the second sialylated MMO is administered to the animal for:
   if subject is bovine: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
   if subject is a pig: 1-18, preferably 1-16, more preferably 1-14, even more preferably 1-12, even more preferably 2-12, even more preferably 3-12, even more preferably 4-12, most preferably 6-12, consecutive weeks;
   if subject is a sheep: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
   if subject is a goat: 1-18, preferably 1-16, more preferably 1-14, even more preferably 1-12, even more preferably 2-12, even more preferably 3-12, even more preferably 4-12, most preferably 6-12, consecutive weeks;
   if subject is a horse: 1-36, preferably 1-32, more preferably 1-30, even more preferably 1-28, even more preferably 1-26, even more preferably 1-18, even more preferably 1-12, even more preferably 2-12, even more preferably 3-12, even more preferably 4-12, most preferably 6-12, consecutive weeks;
   if subject is a camelid: 1-14, preferably 1-12, more preferably 2-12, even more preferably 3-12, even more preferably 4-12, most preferably 6-12, consecutive weeks;
   if subject is a rabbit: 1-12, preferably 2-12, more preferably 3-12, even more preferably 4-12, most preferably 6-12, consecutive weeks;
   if subject is a fish or a crustacean: 1-36, preferably 1-32, more preferably 1-24, even more preferably 1-18, even more preferably 1-12, even more preferably 2-12, even more preferably 3-12, even more preferably 6-12, consecutive months;
   if subject is poultry (but not a turkey): 1-8, preferably 1-7, more preferably 2-7, even more preferably 3-7, most preferably 4-7, consecutive weeks;
   if subject is a turkey: 1-18, preferably 1-16, more preferably 1-14, even more preferably 1-12, even more preferably 2-12, even more preferably 3-12, even more preferably 4-12, most preferably 6-12, consecutive weeks.
101. A method according to any one of embodiments 76 to 100, wherein the first sialylated MMO and the second sialylated MMO are administered to the animal and wherein the ratio between the amount of the first sialylated MMO administered to the animal and the amount of the second sialylated MMO administered to the animal is similar to this ratio as found in the mother's milk of the same species.
102. A method according to any one of embodiments 76 to 101, wherein the first sialylated MMO and the second sialylated MMO are administered to the animal and wherein the amount of the first sialylated MMO administered to the animal is higher than the amount of the second sialylated MMO, preferably the amount of the first sialylated MMO is 1.1 to 40.0, more preferably 1.1 to 35.0, most preferably 1.5 to 35.0, times the amount of the second sialylated MMO administered to the animal.
103. A method according to any one of embodiments 76 to 101, wherein the first sialylated MMO and the second sialylated MMO are administered to the animal and wherein the amount of the first sialylated MMO administered to the animal is higher than the amount of the second sialylated MMO administered to the animal, preferably the amount of the first sialylated MMO administered to the animal is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of the second sialylated MMO administered to the animal.
104. A method according to any one of embodiments 76 to 103, wherein the fucosylated MMO, the first sialylated MMO and the second sialylated MMO are administered to the animal and wherein the amount of the fucosylated MMO administered to the animal is lower than the amount of the first sialylated MMO administered to the animal, preferably the amount of the fucosylated MMO administered to the animal is 1.1 to 10.0, preferably 1.5-7.5, more preferably 2.5-7.5, most preferably 4.0-7.0, times lower than the amount of the first sialylated MMO administered to the animal.
105. A method according to any one of embodiments 76 to 104, wherein the fucosylated MMO, the first sialylated MMO and the second sialylated MMO are administered to the animal and wherein the amount of the fucosylated MMO administered to the animal is lower than the amount of the second sialylated MMO, preferably the amount of the fucosylated MMO is 1.1 to 10.0, preferably 1.5-7.5, more preferably 1.5-5.0, most preferably 2.5-5.0, times lower than the amount of the second sialylated MMO.
106. A method according to any one of embodiments 76 to 105, wherein the second sialylated MMO is part of a composition.
107. A method according to embodiment 106, wherein the second sialylated MMO is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.
108. A method according to embodiment 106 or 107, wherein the second sialylated MMO is present 0.001-0.250 wt. %, preferably 0.001-0.200 wt. %, more preferably 0.001-0.100 wt. %, even more preferably 0.001-0.075 wt. %, even more preferably 0.001-0.050 wt. %, even more preferably 0.005-0.050 wt. %, even more preferably 0.010-0.050 wt. %, even more preferably 0.010-0.030 wt. %, most preferably 0.010-0.020 wt. %, of the total weight of the composition, preferably wherein the animal is a bovine or a horse, more preferably wherein the animal is a calf or a foal, even more preferably wherein the animal is a calf.
109. A method according to embodiment 106 or 107, wherein the second sialylated MMO is present at 0.010-1.000 wt. %, preferably 0.025-0.750 wt. %, even more preferably 0.050-0.750 wt. %, even more preferably 0.050-0.500 wt. %, most preferably 0.050-0.250 wt. %, of the total weight of the composition, preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
110. A method according to embodiment 106 or 107, wherein the second sialylated MMO is present at 0.001-2.500 wt. %, preferably 0.001-1.000 wt. %, more preferably 0.010-1.000 wt. %, even more preferably 0.010-0.750 wt. %, even more preferably 0.010-0.500 wt. %, even more preferably 0.010-0.300 wt. %, most preferably 0.010-0.200 wt. %, of the total weight of the composition, preferably wherein the animal is a fish or a crustacean, more preferably a fish, even more preferably a fry or a fingerling, even more preferably a fingerling.
111. A method according to any one of embodiments 1 to 110, wherein the amount of all sialylated MMOs that are administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated administered to the animal.
112. A method according to any one of embodiments 1 to 110, wherein the amount of the fucosylated MMO that is administered to the animal is lower, preferably 1.1 to 15.0 times lower, more preferably 1.5 to 15.0 times lower, even more preferably 2.5 to 15.0 times lower, even more preferably 5.0 to 15.0 times lower, even more preferably 5.0 to 12.5 times lower, most preferably 5.0 to 10.0 times lower, than the amount of all sialylated MMOs administered to the animal.
113. A method according to any one of embodiments 76 to 112, wherein the amount of all sialylated MMOs in the composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO in the composition.
114. A method according to any one of embodiments 76 to 112, wherein the amount of the fucosylated MMO in the composition is lower, preferably 1.1 to 15.0 times lower, more preferably 1.5 to 15.0 times lower, even more preferably 2.5 to 15.0 times lower, even more preferably 5.0 to 15.0 times lower, even more preferably 5.0 to 12.5 times lower, even more preferably 5.0 to 10.0 times lower, than the amount of all sialylated MMOs in the composition.
115. A method according to any one of embodiments 106 to 114, wherein the amount of the fucosylated MMO in the composition is lower than the amount of the first sialylated MMO.
116. A method according to any one of embodiments 106 to 115, wherein the amount of the fucosylated MMO in the composition is 1.1 to 10.0, preferably 1.5-7.5, more preferably 2.5-7.5, most preferably 4.0-7.0, times lower than the amount of the first sialylated MMO in the composition.
117. A method according to any one of embodiments 106 to 116, wherein the amount of the fucosylated MMO in the composition is lower than the amount of the second sialylated MMO.
118. A method according to any one of embodiments 106 to 117, wherein the amount of the fucosylated MMO in the composition is 1.1 to 10.0, preferably 1.5-7.5, more preferably 2.5-7.5, most preferably 4.0-7.0, times lower than the amount of the second sialylated MMO in the composition.
119. A method according to any one of embodiments 106 to 119, wherein the ratio between the amount of the first sialylated MMO in the composition and the second sialylated MMO in the composition is similar to this ratio as found in the mother's milk of the same species.
120. A method according to any one of embodiments 106 to 119, wherein the amount of the first sialylated MMO in the composition is higher than the amount of the second sialylated MMO.
121. A method according to any one of embodiments 106 to 120, wherein the amount of the first sialylated MMO in the composition is 1.1 to 6.0, preferably 1.1 to 5.0, even more preferably 1.25-4.0, even more preferably 1.25-3.0, even more preferably 1.5 to 3.0, most preferably 1.5-2.5, times higher than the amount of the second sialylated oligosaccharide in the composition.
122. A method according to any one of embodiments 30-34, 62-69, 72, 73, 106-110 and 113 to 121, wherein the composition is not mother's milk.
123. A method according to any one of embodiments 30-34, 62-69, 72, 73, 106-110 and 113 to 122, wherein the composition is a synthetic composition.
124. A method according to any one of embodiments 30-34, 62-69, 72, 73, 106-110 and 113 to 123, wherein the composition is a pharmaceutical composition, optionally further comprising a pharmaceutically acceptable carrier, filler, preservative, solubilizer, diluent, excipient, salt, adjuvant and/or solvent.
125. A method according to any one of embodiments 30-34, 62-69, 72, 73, 106-110 and 113 to 124, wherein the composition is a nutritional composition, optionally further comprising a feed ingredient, wherein the feed ingredient is preferably chosen from the group consisting of:
   a lipid, preferably one or more selected from the group consisting of an oil, fat, ester, monoglyceride, diglyceride, triglyceride and free fatty acid;
   a vitamin, preferably one or more selected from the group consisting of vitamin A, vitamin B, vitamin C, vitamin D, vitamin E and vitamin H, or a derivate thereof;
   an amino acid compound;
   a trace element;
   a mineral, preferably one or more selected from the group consisting of calcium, phosphorus, magnesium, iron, zinc, manganese, copper, sodium, potassium, molybdenum, chromium, selenium and chloride;
   an antioxidant;
   a prebiotic agent, preferably one or more selected from the group consisting of GOS (galacto-oligosaccharide), FOS (fructo-oligosaccharide), inulin and resistant starch;
   a carbohydrate;
   an antimicrobial agent; and
   a protein.
126. A method according to embodiment 125, wherein the nutritional composition is selected from the group consisting of a milk replacer, creep feed, prestarter diet, starter diet, pre-weaning feed, weaning feed and post-weaning feed, preferably selected form the group consisting of a milk replacer, starter diet, creep feed and post-weaning feed, more preferably the nutritional composition is a milk replacer or starter diet, most preferably a milk replacer.

The disclosure more preferably relates to the following specific embodiments:
1. A method to reduce the feed conversion ratio (FCR) of a farm animal, wherein the method comprises the step of administering to a farm animal: (i) a fucosylated mammalian milk oligosaccharide (MMO) and a sialylated mammalian milk oligosaccharide (MMO), (ii) a fucosylated mammalian milk oligosaccharide (MMO) or (iii) a sialylated mammalian milk oligosaccharide (MMO).
2. A method according to embodiment 1, wherein the (i) a fucosylated MMO and a sialylated MMO, the (ii) a fucosylated MMO or the (iii) a sialylated MMO reduce the FCR of the farm animal.
3. Use of (i) a fucosylated MMO and a sialylated MMO, (ii) a fucosylated MMO or (iii) a sialylated MMO to reduce the FCR of a farm animal.
4. A method according to embodiment 1 or 2 or use according to embodiment 3, wherein the farm animal is selected from the group consisting of bovine, pig, sheep, goat, horse, donkey, mule, hinny, camelid (preferably llama or alpaca), rabbit, fish, crustacean and poultry, preferably wherein the camelid is a llama or alpaca.
5. A method according to any one of embodiments 1, 2 or 4 or use according to embodiment 3 or 4, wherein the fucosylated MMO comprises a fucose that is linked to a monosaccharide in an α-1,2-linkage, and wherein the monosaccharide is galactose.
6. A method according to any one of embodiments 1, 2, 4 or 5 or use according to any one of embodiments 3 to 5, wherein the fucosylated MMO is 2′-fucosyllactose.
7. A method according to any one of embodiments 1, 2, 4, 5 or 6 or use according to any one of embodiments 3 to 6, wherein the fucosylated MMO is part of a composition.
8. A method according to any one of embodiments 1, 2, 4, 5, 6 or 7 or use according to any one of embodiments 3 to 7, wherein the sialylated MMO comprises a sialic acid, preferably a N-acetylneuraminic acid (Neu5Ac), that is linked to a monosaccharide in an α-2,3- or an α-2,6-linkage, and wherein the monosaccharide is galactose or N-acetylglucosamine.
9. A method according to any one of embodiments 1, 2, 4, 5, 6, 7 or 8 or use according to any one of embodiments 3 to 8, wherein the sialylated MMO is 3′-sialyllactose (3′SL) or 6′-sialyllactose (6′SL).
10. A method according to any one of embodiments 1, 2, 4, 5, 6, 7, 8 or 9 or use according to any one of embodiments 3 to 9, wherein the sialylated MMO is part of a composition.
11. A method according to any one of embodiments 1, 2, 4, 5, 6, 7, 8, 9 or 10 or use according to any one of embodiments 3 to 10, wherein the fucosylated MMO and the sialylated MMO are administered to the farm animal in two different stages:
   stage 1: administering the sialylated MMO and optionally administering the fucosylated MMO; and
   stage 2: administering the fucosylated MMO, preferably without administering the sialylated MMO.
12. A method according to any one of embodiments 1, 2, 4, 5, 6, 7, 8, 9 or 10 or use according to any one of embodiments 3 to 10, wherein the fucosylated MMO and the sialylated MMO are administered to the farm animal in two different stages:
   stage 1: administering the sialylated MMO and the fucosylated MMO; and
   stage 2: administering the fucosylated MMO, without administering the sialylated MMO.
13. A method according to any one of embodiments 1, 2, 4, 5, 6, 7, 8, 9, 10, 11 or 12 or use according to any one of embodiments 3 to 12, wherein the sialylated MMO is a first sialylated MMO and a second sialylated MMO and optionally a further sialylated MMO.
14. A method according to embodiment 13 or use according to embodiment 13, wherein the first sialylated MMO comprises a sialic acid, preferably a N-acetylneuraminic acid (Neu5Ac), that is linked to a monosaccharide in an α-2,3-linkage, and wherein the monosaccharide is galactose or N-acetylglucosamine.
15. A method according to embodiment 13 or 14 or use according to embodiment 13 or 14, wherein the first sialylated MMO is 3′-sialyllactose (3′SL).
16. A method according to any one of embodiments 13 to 15 or use according to any one of embodiments 13 to 15, wherein the second sialylated MMO comprises a sialic acid, preferably a N-acetylneuraminic acid (Neu5Ac), that is linked to a monosaccharide in an α-2,6-linkage, and wherein the monosaccharide is galactose or N-acetylglucosamine.
17. A method according to any one of embodiments 13 to 16 or use according to any one of embodiments 13 to 16, wherein the second sialylated MMO is 6′-sialyllactose (6′SL).
18. A method to increase the body weight of a farm animal, wherein the method comprises the step of administering to the farm animal a sialylated MMO and a fucosylated MMO.
19. A method according to embodiment 18, wherein the fucosylated MMO and the sialylated MMO are administered to the farm animal in two different stages:
   stage 1: administering the sialylated MMO and optionally administering the fucosylated MMO; and
   stage 2: administering the fucosylated MMO, preferably without administering the sialylated MMO.
20. A method according to embodiment 18, wherein the fucosylated MMO and the sialylated MMO are administered to the farm animal in two different stages:
   stage 1: administering the sialylated MMO and the fucosylated MMO; and
   stage 2: administering the fucosylated MMO without administering the sialylated MMO.
21. A method according to embodiment 19 or 20, wherein the fucosylated MMO is as defined in any one of embodiments 5 to 7.
22. A method according to any one of embodiments 19 to 21, wherein the sialylated MMO is as defined in any one of embodiments 8 to 10 or 13 to 17.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments disclosed herein are to be understood not only in the order and context specifically described in this specification, but to include any order and any combination thereof. Each embodiment as identified herein may be combined together unless otherwise indicated. All publications, patents and patent applications cited in this specification are herein incorporated by reference in their entirety to the same extent as if each individual publication, patent or patent application was specifically and individually indicated to be incorporated by reference in its entirety. Unless specifically stated otherwise, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only and are not intended to imply any particular order for performing the steps (unless specifically stated otherwise).

In this document and in its claims, the verbs “to comprise,” “to have” and “to contain,” and their conjugations are used in their non-limiting sense to mean that items following the word are included, but items not specifically mentioned are not excluded. The verb “to consist essentially of” means that, e.g., a composition as defined herein may comprise additional component(s) than the ones specifically identified, the additional component(s) not altering the unique characteristic of the disclosure. Throughout the document and claims, unless specifically stated otherwise, the verbs “to comprise,” “to have” and “to contain,” and their conjugations, may be preferably replaced by “to consist” (and its conjugations) or “to consist essentially of” (and its conjugations) and vice versa. In addition, reference to an element by the indefinite article “a” or “an” does not exclude the possibility that more than one of the elements is present, unless the context clearly requires that there be one and only one of the elements. The indefinite article “a” or “an” thus usually means “at least one.” The word “about” or “approximately” or “around” when used in association with a numerical value (e.g., about 10) preferably means that the value may be the given value (of 10) more or less 10%, preferably 5%, more preferably 1% of the value.

Throughout the description and claims, unless specifically stated otherwise, the expression “from x to y,” wherein x and y represent numerical values, refers to a range of numerical values wherein both x and y are included and wherein x represents the lowest value and y represents the highest value. Hence, both x and y are included in the range.

The terms “LNT II,” “LNT-II,” “LN3,” “lacto-N-triose II,” “lacto-N-triose II,” “lacto-N-triose,” “lacto-N-triose” and “GlcNAc-β1,3-Gal-β1,4-Glc” are used interchangeably.

The terms “LNT,” “lacto-N-tetraose,” “lacto-N-tetraose” and “Gal-β1,3-GlcNAc-β1,3-Gal-β 1,4Glc” are used interchangeably.

The terms “LNnT,” “lacto-N-neotetraose,” “lacto-N-neotetraose,” “neo-LNT” and “Galβ1-4GlcNAcβ1-3Galβ1-4Glc” are used interchangeably.

The terms “2′ fucosyllactose,” “2′-fucosyllactose,” “α-1,2-fucosyllactose,” “a 1,2 fucosyllactose,” “α-1,2-fucosyllactose,” “a 1,2 fucosyllactose,” “Fuc-α1,2-Gal-β1,4-Glc,” “2FL” and “2′FL” are used interchangeably.

The terms “3-fucosyllactose,” “α-1,3-fucosyllactose,” “a 1,3 fucosyllactose,” “α-1,3-fucosyllactose,” “a 1,3 fucosyllactose,” “Gal-β1,4-(Fuc-α1,3-)Glc,” 3FL” and “3-FL” are used interchangeably.

The terms “difucosyllactose,” “di-fucosyllactose,” “lactodifucotetraose,” “2′,3-difucosyllactose,” “2′,3 difucosyllactose,” “α-2′,3-fucosyllactose,” “a 2′,3 fucosyllactose,” “Fuc-α1,2-Gal-β1,4-(Fuc-α1,3-)Glc,” “DFLac,” “2′,3 diFL,” “DFL,” “DiFL” and “diFL” are used interchangeably.

The terms “LNFP-I,” “lacto-N-fucopentaose I,” “LNFP I,” “LNF I OH type I determinant,” “LNF I,” “LNF1,” “LNF 1,” “Blood group H antigen pentaose type 1” and “Fuc-α1,2-Gal-β1,3-GlcNAc-β1,3-Gal-β1,4-Glc” are used interchangeably.

The terms “GalNAc-LNFP-I,” “blood group A antigen hexaose type I,” and “GalNAc-α1,3-(Fuc-α1,2)-Gal-β1,3-GlcNAc-β1,3-Gal-β1,4-Glc” are used interchangeably.

The terms “Gal-LNFP-I,” “blood group B antigen hexaose type I” and “Gal-α1,3-(Fuc-α1,2)-Gal-β1,3-GlcNAc-β1,3-Gal-β1,4-Glc” are used interchangeably.

The terms “LNFP-II,” “lacto-N-fucopentaose II” and “Gal-β1,3-(Fuc-α1,4)-GlcNAc-β1,3-Gal-β1,4-Glc” are used interchangeably.

The terms “LNFP-III,” “lacto-N-fucopentaose III” and “Gal-β1,4-(Fuc-α1,3)-GlcNAc-β1,3-Gal-β1,4-Glc” are used interchangeably. The terms “LNFP-V,” “lacto-N-fucopentaose V” and “Gal-β1,3-G1cNAc-β1,3-Gal-β1,4-(Fuc-α1,3)-Glc” are used interchangeably.

The terms “LNDFH I,” “Lacto-N-difucohexaose I,” “LNDFH-I,” “LDFH I,” “Le^{b}-lactose,” “Lewis-b hexasaccharide” and “Fuc-α1,2-Gal-β1,3-[Fuc-α1,4]-GlcNAc-β1,3-Gal-β1,4-Glc” are used interchangeably.

The terms “LNDFH II,” “Lacto-N-difucohexaose II,” “Lewis a-Lewis x,” “LDFH II” and “Fuc-α1,4-(Gal-β1,3)-GlcNAc-β1,3-Gal-β1,4-(Fuc-α1,3)-Glc” are used interchangeably.

The terms “lewis b-lewis x” and “Fucα1,4-[Fuc-α1,2-Galβ1,3]-GlcNAc-β1,3-Gal-β1,4-[Fuc-α1,3]-Glc” are used interchangeably.

The terms “MFLNH III,” “monofucosyllacto-N-hexaose-III” and “Gal-β1,4-[Fuc-α1,3]-GlcNAc-β1,6-[Gal-β1,3-GlcNAc-β1,3]-Gal-β1,4-Glc” are used interchangeably.

The terms “DFLNH (a),” “difucosyllacto-N-hexaose (a)” and “Gal-β1,4-[Fuc-α1,3]-GlcNAc-β1,6-[Fuc-α1,2-Gal-β1,3-GlcNAc-β1,3]-Gal-β1,4-Glc” are used interchangeably.

The terms “DFLNH,” “difucosyllacto-N-hexaose” and “Gal-β1,4-[Fuc-α1,3]-GlcNAc-β1,6-[Fuc-α1,4-[Gal-β1,3]-GlcNAc-β1,3]-Gal-β1,4-Glc” are used interchangeably.

The terms “TFLNH,” “trifucosyllacto-N-hexaose” and “Gal-β1,4-[Fuc-α1,3]-GlcNAc-β1,6-[Fuc-α1,4-[Fuc-α1,2-Gal-β1,3]-GlcNAc-β1,3]-Gal-β1,4-Glc” are used interchangeably.

The terms “LNnFP I,” “Lacto-N-neofucopentaose I” and “Fuc-α1,2-Gal-β1,4-GlcNAc-β1,3-Gal-β1,4-Glc” are used interchangeably.

The terms “LNFP-VI,” “LNnFP V,” “lacto-N-neofucopentaose V” and “Gal-β1,4-GlcNAc-β1,3-Gal-β1,4-(Fuc-α1,3)-Glc” are used interchangeably.

The terms “LNnDFH,” “Lacto-N-neoDiFucohexaose,” “Lewis x hexaose” “Gal-β1,4-(Fuc-α1,3)-GlcNAc-β1,3-Gal-β1,4-(Fuc-α1,3)-Glc” are used interchangeably.

The terms “2′-fucosyllacto-N-biose,” “2′FLNB” and “Fuc-α1,2-Gal-β1,3-GlcNAc” are used interchangeably.

The terms “4-fucosyllacto-N-biose,” “4FLNB” and “Fuc-α1,4-[Gal-β1,3-]GlcNAc” are used interchangeably.

The terms “difucosyllacto-N-biose,” “diFLNB” and “Fuc-α1,4-[Fuc-α1,2-Gal-β1,3-]GlcNAc” are used interchangeably.

The terms “2′-fucosyl-N-acetyllactosamine,” “2′FlacNAc” and “Fuc-α1,2-Gal-β1,4-GlcNAc” are used interchangeably.

The terms “3-fucosyl-N-acetyllactosamine,” “3FlacNAc” and “Gal-β1,4-(Fuc-α1,3-)GlcNAc” are used interchangeably.

The terms “difucosyl-N-acetyllactosamine,” “diFlacNAc” and “Fuc-α1,2-Gal-β1,4-[Fuc-α1,3-]GlcNAc” are used interchangeably.

The terms “3′ sialyllactose,” “3′-sialyllactose,” “α-2,3-sialyllactose,” “a 2,3 sialyllactose,” “α-2,3-sialyllactose,” “a 2,3 sialyllactose,” “3SL,” “Sia-α2,3-Gal-β1,4-Glc” and “3′SL” are used interchangeably.

The terms “6′ sialyllactose,” “6′-sialyllactose,” “α-2,6-sialyllactose,” “a 2,6 sialyllactose,” “α-2,6-sialyllactose,” “a 2,6 sialyllactose,” “6SL,” “Sia-α2,6-Gal-β1,4-Glc” and “6′SL” are used interchangeably.

The terms “3,6-disialyllactose” and “Neu5Ac-α2,3-Neu5Ac-α2,6-Gal-β1,4-Glc” are used interchangeably.

The terms “6,6′-disialyllactose” and “Neu5Ac-α2,6-Neu5Ac-α2,6-Gal-β1,4-Glc” are used interchangeably.

The terms “8,3-disialyllactose” and “Neu5Ac-α2,8-Neu5Ac-α2,3-Gal-β1,4-Glc” are used interchangeably.

The terms “3′S-2′FL,” “3′-sialyl-2′-fucosyllactose” and “Neu5Ac-α2,3-[Fuc-α1,2-]Gal-β1,4-GlC” are used interchangeably.

The terms “6′S-2′FL,” “6′-sialyl-2′-fucosyllactose” and “Neu5Ac-α2,6-[Fuc-α1,2-]Gal-β1,4-GlC” are used interchangeably.

The terms “3′S-3-FL,” “3′-sialyl-3-fucosyllactose” and “Neu5Ac-α2,3-Gal-β1,4-[Fuc-α1,3]Glc” are used interchangeably.

The terms “6′S-3-FL,” “6′-sialyl-3-fucosyllactose” and “Neu5Ac-α2,6-Gal-β1,4-[Fuc-α1,3]Glc” are used interchangeably.

The terms “LSTa,” “LS-Tetrasaccharide a,” “Sialyl-lacto-N-tetraose a,” “sialyllacto-N-tetraose a” and “Neu5Ac-α2,3-Gal-b1,3-GlcNAc-b1,3-Gal-b1,4-Glc” are used interchangeably.

The terms “LSTb,” “LS-Tetrasaccharide b,” “Sialyl-lacto-N-tetraose b,” “sialyllacto-N-tetraose b” and “Gal-b1,3-(Neu5Ac-α2,6)-GlcNAc-b1,3-Gal-b1,4-Glc” are used interchangeably.

The terms “LSTc,” “LS-Tetrasaccharide c,” “Sialyl-lacto-N-tetraose c,” “sialyllacto-N-tetraose c,” “sialyllacto-N-neotetraose c” and “Neu5Ac-α2,6-Gal-b1,4-GlcNAc-b1,3-Gal-b1,4-Glc” are used interchangeably.

The terms “LSTd,” “LS-Tetrasaccharide d,” “Sialyl-lacto-N-tetraose d,” “sialyllacto-N-tetraose d,” “sialyllacto-N-neotetraose d” and “Neu5Ac-α2,3-Gal-b1,4-GlcNAc-b1,3-Gal-b1,4-Glc” are used interchangeably.

The terms “3′-sialyllacto-N-biose,” “3′SLNB” and “Neu5Ac-α2,3-Gal-b1,3-GlcNAc” are used interchangeably.

The terms “6′-sialyllacto-N-biose,” “6′SLNB” and “Neu5Ac-α2,6-Gal-b1,3-GlcNAc” are used interchangeably.

The terms “monofucosylmonosialyllacto-N-octaose,” “sialyl Lewis a,” “sialyl Lea,” “5-acetylneuraminyl-(2-3)-galactosyl-(1-3)-(fucopyranosyl-(1-4))-N-acetylglucosamine” and “Neu5Ac-α2,3-Gal-β1,3-[Fuc-α1,4]-GlcNAc” are used interchangeably.

The terms “3′-sialyllactosamine,” “3′SLacNAc” and “Neu5Ac-α2,3-Gal-b1,4-GlcNAc” are used interchangeably.

The terms “6′-sialyllactosamine,” “6′SLacNAc” and “Neu5Ac-α2,6-Gal-b1,4-GlcNAc” are used interchangeably.

The terms “sialyl Lewis x,” “sialyl Lex,” “5-acetylneuraminyl-(2-3)-galactosyl-(1-4)-(fucopyranosyl-(1-3))-N-acetylglucosamine” and “Neu5Ac-α2,3-Gal-β1,4-[Fuc-α1,3-]GlcNAc” are used interchangeably.

The term “cultivation” refers to the culture medium wherein the cell is cultivated or fermented, the cell itself, and the saccharide(s) that is/are produced by the cell in whole broth, i.e., inside (intracellularly) as well as outside (extracellularly) of the cell.

The term “clarifying” refers to the act of treating an aqueous solution to remove suspended particulates and contaminants from the production process, like, e.g., cells, cell components, insoluble metabolites and debris, that could interfere with the eventual purification of the oligosaccharide(s) of interest. Such treatment can be carried out in a conventional manner by centrifugation, flocculation, flocculation with optional ultrasonic treatment, gravity filtration, microfiltration, foam separation or vacuum filtration (e.g., through a ceramic filter that can include a Celite™ filter aid).

### EXAMPLES

The disclosure will be described in more detail in the examples. The following examples will serve as further illustration and clarification of the disclosure and are not intended to be limiting in any way.

### Example 1: 2′FL

2′-fucosyllactose (2′FL) was recombinantly produced in E. coli and purified as described in Examples 10, 13 and 14 of WO 2022/034079, and subsequently spray dried as described in Example 21 of WO 2022/034079 to obtain 2″FL powder (purity 89.6%).

### Example 2: 3′SL and 6′SL

3′sialyllactose (3′SL) and 6′sialyllactose (6′SL) were recombinantly produced in E. coli and purified as described in WO 2022/034079 (Examples 11, 13 and 14; the E. coli strains are described in Example 3 of WO 2018/122225 for 6′SL and Example 7 of WO 2018/122225 for 3′SL wherein an α-2,3-sialyltransferase from Pasteurella multocida, i.e., amino acids 1-268 of UniprotID Q9CLP3 sequence v1, was used) and subsequently spray dried as described in Example 21 of WO 2022/034079 to obtain 3′SL powder (purity 98.4%) and 6′SL powder (purity 98.6%).

### Example 3: Trial in Calves

### Animals

Holstein Friesian calves (2- to 3-week-old, 95% male) were transported from different farms to the experimental facility of Denkavit (Voorthuizen, The Netherlands) where the animals were reared. The calves were randomly and gender-equally divided in four groups based on experimental calf milk replacer (CMR): group A (placebo—standard CMR powder of the farm), group B (standard CMR powder supplemented with 0.005% dry wt. 2′FL), group C (standard CMR powder supplemented with 0.025% dry wt. 3′SL and 0.015% w/w 6′SL) and group D (standard CMR powder supplemented with 0.005% dry wt. 2′FL, 0.025% dry wt. 3′SL and 0.015% dry wt. 6′SL). The calf milk replacer was administered as a liquid to the calves (water was added to the CMR powder to obtain 150 grams CMR powder per liter liquid CMR). Each group involved 100 calves, assigned into four rows (25 calves per row) of two compartment blocks. Each row contained five pens, where four to six calves were living in one pen.

### Diet

Starting from the day of arrival, calves received standard calf milk replacer (CMR) during a period of 9 weeks. Group A only received the CMR, while the CMR supplied to groups B, C and D was supplemented with MMOs as outlined in the section titled “Animals.” Calf starter (concentrates) and straw were introduced gradually from day 6 till the end of the experiment (12 weeks) while calves had access to corn silage at week 11 and 12. Feeds were provided ad libitum to calves twice a day in the morning and evening and were daily recorded based on rows (concentrates, straw, corn silage) or groups (CMR) consumption.

Major components of standard CMR and starter diets are presented in Table 1.

TABLE 1
Major components of calf's standard diets

| | Protein | Fat | Carbohydrate | Crude fiber | Ash |
|---|---|---|---|---|---|
| | (dry | (dry | (dry | (dry | (dry |
| Diet | wt. %) | wt. %) | wt. %) | wt. %) | wt. %) |
| Calf milk | 22 | 18 | 49 | | |
| replacer | Whey powder, vegetable oils (palm, coco, rapeseed), | | | | |
| | hydrolyzed wheat gluten, dextrose, premix | | | | |
| Calf | 17.5 | 2.2 | | 5.8 | 6.9 |

| | |
|---|---|
| starter | Corn, soybean meal, beet pulp, barley, wheat, whey powder, |
| | sugar, calcium carbonate, lignocellulose, premix |

### Health, Safety and Performance Observation

Calves were followed up by the farm staff since arrival until 12 weeks. Fecal consistency in scale 1-4 (Larson et al., 1977, J. Dairy Science, 60(6): p. 989-991) was recorded daily for the first ten days since arrival. Number of mortalities and signs of adverse effects were registered daily. The animals were treated prophylactically or metaphylactically (herd treatment) with 1 g oxytetracycline 100% (at day 4), 0.5 g Doxylin 50% WSP and 0.8 g Bromhexine 2% (at day 13), and 3.1 ml Tilmicosol 250 mg/ml (at day 24) to prevent and/or treat acute lung inflammation. Individual treatments were followed by remarks of the farm staff and diagnostics of farm veterinary practitioners. Disease incidences and medicinal usage based on individual treatments were recorded to compare between treatment groups. The incidence of diarrhea was 0% in all groups at the start of the study, and 1% (%; group A), 0% (group B), 1% (group C) and 2% (group D) after 12 weeks. Live weight of the animals was monitored at arrival (d1), two weeks (W2), six weeks (W6), ten weeks (W10) and 12 weeks (W12) since arrival. Feed conversion ratio (FCR) of each period was computed based on the input of daily weight gain and daily feed intake.

### Fecal Sampling

Stools were collected directly from recta of six calves in one selected pen upon rectal inducing at day 29 (W5) and day 57 (W9). The fecal samples (approximately 1 g) were immediately snap frozen using isopropanol-dried ice bath for 1 minute before they were transferred onto dried ice for transportation to a long-term storage at −80° C.

### Total Microbial DNA Extraction and Shotgun Metagenomics

Total fecal DNA was extracted from 200 mg feces while total upper respiratory tract microbial DNA were extracted from 1 ml DNA shield reagent containing the nasal pharyngeal swab. ZymoBIOMICS™ DNA miniprep kit (Zymo Research) in combination with a FASTPREP® 24 device was used to extract and purify the microbial DNA as instructed by the manufacturer. Purified total DNA were eluted in 100 μl and were quickly checked for the concentration and quality using Nanodrop (Implen NANOPHOTOMETER®) before storage at −20° C.

Total DNA were submitted to Eurofins (GATC, Germany) to perform metagenomics using their INVIEW metagenome platform. Briefly, total DNA were first fragmented, then ligated with adapter for library preparation. The sequencing was done using Illumina paired end sequencing technology (2×150 bp), guaranteed for 20 million read pairs (40 million reads). Reads were quality-checked with fastqc (bioinformatics.babraham.ac. uk/projects/fastqc/) and trimmed with Trimmomatic (Bolger et al., 2014, Bioinformatics, 30(15): p. 2114-2120; usadellab.org/cms/?page=trimmomatic). Bovine reads were removed by mapping to the cow reference database Bos_taurus_UMD_3.1.1 using BMTagger (bioconda.github.io/recipes/bmtagger/README.html). Profiling of the taxonomic composition and the functional potential of microbial communities was done using Metaphlan (Beghini et al., 2021, Elife 10: e65088; /huttenhower.sph.harvard.edu/humann/).

### Statistical Analysis

Prior to analysis, the data of live weight and FCR were checked for outliers. A data point was considered an outlier if it could reasonably be expected that observed trail results were due to other factors than the treatment, e.g., due to sickness. An ANCOVA model as below was used to analyze the live weight data (R version 4.1.2.).

$${y}_{ijk} = μ + {treatment}_{i} + {placement}_{j} + treatment * {placement}_{ij} + weight\_ 0 k + treatment * weight\_ {0}_{ik} + {ε}_{ijk}$$

$$Where :$$

$${y}_{ijk} : dependent variable$$

$$μ : average response of y on the independent variables$$

$${treatment}_{i} : treatment groups ( 4 )$$

$${placement}_{j} : factor for barn effects ( 4 )$$

$$weight\_ {0}_{k} : starting weight of the calves$$

$$n {ε}_{ijk} : error term$$

Differences among means with P<0.05 were accepted as significant. Differences among means with 0.05<P<0.10 were accepted as tendency. Multiple comparisons were done with Tukey.

Other performance data were analyzed using a mixed linear model using treatments as fixed and Row as random term using 1me in the nlme package.

Microbiome data was normalized in cpm (counts per million).

The treatment effect of MMOs in the microbiome data was evaluated per timepoint with Maaslin2 (Mallick et al., 2021, PLoS Computational Biology 17(11): e1009442) using AST transformation and adding compartment as fixed effect. For visualization in the tables a score was calculated equal to −log 10(pvalue)*sign(coef). Spearman correlation was used to evaluate the relation between microbiome and FCR using the cor.test function in the stats package in R.

### Results—Feed Conversion Ratio (FCR)

In terms of safety, there were no adverse effects from the MMOs as registered by the farm staff and hence considered safe for the current target animals. During the 12 weeks trial, 7/400 calves died with no significant difference in mortality between the different groups.

Calves (2-3 weeks old) started at the farm with a body weight of around 45.5 kg (Week 0) in all groups. After two weeks, there was not much difference in weight between the groups (i.e., groups A-D). In contrast, since week 6, all MMOs-supplemented calves (i.e., groups B-D) became heavier than the companions eating the control diet (i.e., group A) as summarized in Table 2. In contrast to weight gain, there is no significant difference in feed intake between the groups (Table 2).

TABLE 2
Performance of all calves during the trial (12 weeks).

| | | | | |
|---|---|---|---|---|
| | Group A | Group B | Group C | Group D |
| Index | (placebo) | (2′FL) | (3′SL/6′SL) | (2′FL/3′SL/6′SL) |

Live weight (kg ± SD)

| | | | | |
|---|---|---|---|---|
| W 0 | 45.5 ± 3.1 | 45.6 ± 2.8 | 45.6 ± 2.7 | 45.5 ± 2.8 |
| W 2 | 53.4 ± 3.5 | 54.0 ± 3.3 | 53.9 ± 2.9 | 53.9 ± 3.7 |
| W 6 | 78.3 ± 8.2 | 80.7 ± 7.1 | 81.0 ± 6.6 | 81.2 ± 7.3 |
| W 10 | 112.6 ± 13.0 | 116.2 ± 11.0 | 115.2 ± 11.6 | 116.0 ± 12.7 |
| W 12 | 132.0 ± 14.5 | 136.8 ± 12.7 | 135.6 ± 12.9 | 136.9 ± 14.0 |

Average daily gain (g/day ± SD)

| | | | | |
|---|---|---|---|---|
| ADG W 0-W 2 | 574.3 ± 142.6 | 600.1 ± 155.0 | 593.9 ± 150.9 | 600.4 ± 157.7 |
| ADG W 2-W 6 | 886.8 ± 208.6 | 954.9 ± 176.8 | 969.3 ± 169.4 | 984.7 ± 159.3 |
| ADG W 6-W 10 | 1218.6 ± 233.1 | 1267.9 ± 202.2 | 1232.1 ± 218.5 | 1261.0 ± 191.9 |
| ADG W 10-W 12 | 1409.8 ± 259.9 | 1484.6 ± 260.7 | 1467.0 ± 238.9 | 1473.2 ± 245.9 |
| ADG W 0-W 12 | 1043.1 ± 165.8 | 1099.5 ± 141.7 | 1085.1 ± 145.9 | 1100.1 ± 151.1 |

Average feed intake (kg ± SD)

| | | | | |
|---|---|---|---|---|
| CMR | 41.6 | 42.3 | 42.0 | 42.5 |
| Concentrates | 130.3 ± 2.2 | 131.6 ± 6.2 | 131.4 ± 8.6 | 131.0 ± 6.8 |
| Straw | 15.8 ± 0.2 | 16.0 ± 0.8 | 16.0 ± 1.0 | 15.9 ± 0.9 |
| Corn silage | 14.6 ± 0.2 | 14.8 ± 1.3 | 15.1 ± 0.7 | 14.7 ± 1.5 |

Feed conversion ratio (FCR)

| | | | | |
|---|---|---|---|---|
| FCR W 0-W 2 | 1.42 | 1.34 | 1.34 | 1.39 |
| FCR W 2-W 6 | 1.75 | 1.67 | 1.62 | 1.58 |
| FCR W 0-W 6 | 1.63 | 1.53 | 1.52 | 1.51 |
| FCR W 0-W 10 | 2.09 | 2.01 | 2.00 | 1.99 |
| FCR W 6-W 10 | 2.59 | 2.51 | 2.56 | 2.50 |
| FCR W 10-W 12 | 3.49 | 3.44 | 3.37 | 3.28 |
| FCR W 6-W 12 | 2.88 | 2.76 | 2.83 | 2.75 |
| FCR W 0-W 12 | 2.40 | 2.28 | 2.30 | 2.26 |

CMR = calf milk replacer; W 0 = week 0; W 2 = week 2; W 6 = week 6; W 10 = week 10; W 12 = week 12.

Although consuming a similar amount of feed, calves grew differently depending on whether or not they received MMO supplementation and depending on what type of MMO they received. As compared to the control (i.e., placebo), all MMO-supplemented calves showed a decreased feed conversion ratio (FCR) (P-value=0.058). An improvement of 10 to 14 points means saving 1 ton feed for the same 10 tons of animal body weight.

### Results—Fecal Microbiota and the Correlation Between Fecal Microbiota and the FCR

Several beneficial bacterial species were promoted in the microbiota of the MMO-treated calves. In contrast, potential pathogenic bacteria, namely Atopobium deltae or Treponema succinfaciens, or pro-inflammatory species, namely Prevotella copri or Bacteroides coprophilus, were inhibited in the MMO-treated calves as compared to the placebo calves. Correlations between the abundance of the bacteria with the FCR at W6 and W12 showed negative association between beneficial bacteria and the FCR value, meaning the bacteria support feed efficiency of the calves (see Table 3). Oppositely, the correlation between the potential pathogenic bacteria or the pro-inflammatory bacteria with the FCR value were positive, indicating worsening feed efficiency (see Table 3).

The results suggest that MMO supplementation might support gut health of the calves by promoting beneficial bacteria while inhibited potential pathogenic or pro-inflammatory bacteria so that the feed efficiency is improved.

TABLE 3
Significant changes in counts per million (cpm) of bacteria species
in fecal microbiota of calves in MMO-treated groups (group B,
C and D) versus the ones of the placebo calves (group A) and
the correlation coefficient (R) of the bacteria species with
the FCR at W 6 and the FCR at W 12 (P-value < 0.05).

| | | | | | |
|---|---|---|---|---|---|
| | | | | R | R |
| | | | Group D | (FCR_{—} | (FCR_{—} |
| | Group B | Group C | (2FL/3SL/ | W 0_{—} | W 0_{—} |
| Species | (2FL) | (3SL/6SL) | 6SL) | W 6) | W 12) |

T1 (W 5)

| | | | | | |
|---|---|---|---|---|---|
| Lactobacillus | | | 1.67 | | −0.78 |
| johnsonii | | | | | |
| Lactobacillus | | | 1.78 | | −0.6 |
| reuteri | | | | | |
| Eubacterium | 1.35 | | | | −0.5 |
| rectale | | | | | |
| Atopobium | −1.44 | −1.48 | | 0.37 | |
| deltae | | | | | |
| Prevotella | −1.68 | | −1.79 | 0.41 | |
| copri | | | | | |

T2 (W 9)

| | | | | |
|---|---|---|---|---|
| Coprococcus | | 1.58 | | −0.75 |
| eutactus | | | | |
| Ruminococcus | 1.65 | | | −0.63 |
| bromii | | | | |
| Bacteroides | 1.35 | | | −0.63 |
| pectinophilus | | | | |
| Fusicatenibacter | | 1.39 | | −0.62 |
| saccharivorans | | | | |
| Blautia wexlerae | | | 2.32 | −0.55 |
| Gemmiger | | | 1.33 | −0.5 |
| formicilis | | | | |
| Treponema | | −1.65 | | 0.55 |
| succinifaciens | | | | |
| Bacteroides | −1.56 | | | 0.63 |
| coprophilus | | | | |

### Example 4: Trial in Piglets

### Animals and Trial Design

The in vivo experiment was carried out at the commercial pig farm “Degroote Luc” (Bissegem, Belgium).

The experimental design had two treatments:
“Ti” serves as control, i.e., a placebo (water) was drenched during the first 4 days of life, and subsequently a commercial milk replacer as a liquid and creep feed was offered until weaning,
“T2,” a 100 w/w solution of 2′FL (it is referred to Example 1) in water was drenched during the first 4 days of life, and subsequently a commercial milk replacer supplemented with 2′FL (warm water was added to 250 g of milk powder to reach 25.000 (w/v)) and creep feed supplemented with 2′FL (10% w/w 2′FL) was offered until weaning. The composition of milk replacer and creep feed are detailed in Table 4.

TABLE 4
Feed ingredients and nutrient composition
of pig milk replacer and creep feed.

| | Milk replacer | Creep feed |
|---|---|---|
| | (d 4-d 22) | (d 14-d 22) |
| | (dry wt. %) | (wt. %) |
| Ingredients | | |
| WHEY POWDER | x | x |
| DEXTROSE | x | x |
| COCONUT OIL | x | x |
| WHEAT GLUTEN | x | |
| BLOOD PLASMA | x | x |
| SOYBEAN OIL | x | x |
| INACTIVATED YEAST CELL | x | |
| WALLS | | |
| EXTRUDED SOYBEAN | | x |
| MIDDLINGS | | |
| EXTRUDED WHEAT | | x |
| FLOWER | | |
| TOASTED SOYBEAN | | x |
| OAT FLAKES | | x |
| EXTRUDED CORN | | x |
| BARLEY | | x |
| SUGAR | | x |
| SUNFLOWER OIL | | x |
| WHEAT PROTEIN MEAL | | x |
| LACTOSE | | x |
| SALMON OIL | | x |
| WHEAT MIDDLINGS | | x |
| SODIUM CHLORIDE | | x |
| SODIUM BICARBONATE | | x |
| CALCIUMTRIPHOSPHATE | | x |
| ESTERIFIED MCFA | | x |
| BUTYRIC ACID SALTS | | x |
| PALM KERNEL OIL AND | | x |
| COCOS FATTY ACIDS | | |
| ROSMARY EXTRACT | | x |
| CURCUMA EXTRACT | | x |
| Calculated composition | | |
| ME MJ | 16.7 | — |
| Dry matter % | 95 | 90 |
| Crude protein % | 20 | 19 |
| Crude fat % | 12 | 10 |
| Crude ash % | 7 | 6.5 |
| Crude fiber % | 0.04 | 2 |
| Lysine % | 1.80 | 1.6 |
| Methionine % | 0.55 | 0.5 |
| Calcium % | 0.64 | 0.6 |
| Phosphor % | 0.53 | 0.6 |
| Sodium % | 0.60 | 0.38 |

Within 6 hours after birth of all piglets within a litter, piglets were tagged, weighed and ranked according to live weight. This was d0 of the study. Then, the first drenching was given, i.e., the heaviest piglet in the pen receives Ti, the next piglet T2 and so forth, until 12 piglets of that litter were drenched. In the next litter, the order of treatment application was alternated. This practice was done in 16 litters, and drenching was repeated on d1, d2 and d3. At the d4, 128 piglets were allocated to 16 pens with 6 piglets each, in the nursery unit. Ti was applied in 8 pens, and T2 was offered in 8 remaining pens. Piglets were allocated to pens according to litter of origin (from each of the 16 litters; 6 vital, normal to heavy weight piglets, body weight ≥1.7 kg, were taken and half of the piglets were allocated to a pen from T1—those receiving T1 in the first 3 days of life, and others to a pen from T2—those receiving T2 in the first 3 days of life), and to body weight and sex. Piglets were offered the liquid milk replacer through a cup connected to a mixing-tubing system and had free access to milk at all times. Milk intake was accurately measured at pen level. From d14 on, in addition, piglets were offered a creep feed (solid feed in feeder pan). Piglets were weighed at d0, d4, d14 and d22 (day of weaning).

All pigs were checked twice daily for general health during the whole experimental period. Observations were carefully recorded in a log book.

### Fecal Sampling and Microbial 16S Sequencing

On d22, from one piglet per pen (piglet with median weight at weaning) a fresh feces sample was collected and stored at −80° C. Total fecal DNA of 16 selected piglets (1 per pen) were extracted from 200 mg feces. ZymoBIOMICS™ DNA miniprep kit (Zymo Research) in combination with a FASTPREP® 24 device was used to extract and purify the microbial DNA as instructed by the manufacturer. Purified total DNA were eluted in 100 μl and were quickly checked for the concentration and quality using Nanodrop (Implen NANOPHOTOMETER®) before storage at −20° C.

Total DNA were submitted to Eurofins (GATC, Germany) to perform 16S rRNA sequencing using their INVIEW microbiome high specificity package. Briefly, total DNA were first amplified the hyper-variable regions 16S and integrated the barcodes through PCR. The PCR products were purified, then ligated with adapter for library preparation. The sequencing was done using PacBio sequencing technology, guaranteed for 25000 reads, mean read length 15,000 bp.

Amplicon analysis was performed using Mothur pipeline (mothur.org). Processing of the raw sequencing data was done according to the Mothur standard operating procedure (mothur.org/wiki/MiSeq_SOP). Alignment was accomplished using DAIRYdb 16S rRNA gene database (GitHub—marcomeola/DAIRYdb). Sequences were classified and any sequences classified as Chloroplast, Mitochondria, unknown, Archaea, or Eukaryota were removed from the data set. Preclustering of the sequences and removal of chimeric sequences using a Mothur-formatted version of the Ribosomal Database Project (RDP) training set and uchime, based on the Mothur protocol. Sequences were classified into Operational Taxonomic Units (OTUs) of ≥97% sequence identity. Singleton and doubleton reads were removed.

### Statistical Analysis

Data was collected with respect to piglet performance (body weight, average daily gain (ADG), average daily feed intake and milk replacer for periods d0-4, d4-14, d14-22, d4-22, d0-22, total creep feed intake for period d14-22 and feed conversion for all periods.

Data were analyzed using appropriate linear mixed model with treatment as fixed effect (T1 vs. T2) and litter as random effect, and body weight at d0 as co-variate if P<0.05. Piglet was used as experimental unit for growth data, whereas pen was the experimental unit for other records.

Data statistics of 16S data and figure plotting were performed in RStudio (rstudio.com/). Microbiome data was normalized in relative abundance or cpm (counts per million). The treatment effect of 2′FL in the microbiome abundance data was evaluated on normalized relative abundance (in %) using Mann-Whitney U test.

### Results—2′FL Improved Feed Efficiency of Pre-Weaning Piglets

Performance data are presented in Table 5. Overall pre-weaning growth was 181 g/d, resulting in 5.44 kg body weight at weaning. There were no significant differences between 2′FL treated and the placebo groups in body weight, ADG, milk intake, and creep feed intake during 22 days of pre-weaning phase although piglets in the treated group were slightly heavier but consumed less milk and feed than the control ones. In other words, there is a trend that the FCR is reduced in the treated group during 22 days of pre-weaning phase). Interestingly, FCR during day 14 to 22 was lower in the 2′FL-treated group (P=0.055) in case of milk intake and even lower (P=0.038) if creep feed was also involved. That means 2′FL supplementation significantly improved feed efficiency of piglets since the time they started eating creep feed.

TABLE 5
Effect of 2′FL (1% w/w) supplementation in the pre-weaning
period by means of drenching (d 0-4) and via milk replacer
(d 4-22) on body weight and growth (n = 42), feed
intake (milk replacer and creep feed) and feed conversion.

| | Placebo | 2′FL-treated | SEM | P-value |
|---|---|---|---|---|
| Body weight (g) | | | | |
| d 0 | 1539 | 1536 | 19.9 | 0.839 |
| d 4 | 2045 | 2087 | 32.6 | 0.369 |
| d 14 | 3517 | 3524 | 65.6 | 0.946 |
| d 22 | 5317 | 5524 | 141.0 | 0.306 |
| ADG (g/d) | | | | |
| d 0-4 | 129 | 139 | 8.1 | 0.363 |
| d 4-14 | 150 | 150 | 5.8 | 0.950 |
| d 14-22 | 225 | 250 | 11.7 | 0.134 |
| d 4-22 | 184 | 196 | 7.1 | 0.253 |
| d 0-22 | 175 | 185 | 6.5 | 0.297 |
| ADFI milk (g DM/d) | | | | |
| d 4-14 | 156 | 149 | 8.3 | 0.555 |
| d 14-22 | 268 | 270 | 11.0 | 0.885 |
| d 4-22 | 205 | 202 | 8.2 | 0.834 |
| ADFI creep feed (g DM/d) | | | | |
| d 14-22 | 26.7 | 23.8 | 1.95 | 0.304 |
| FCR (g milk DM/g) | | | | |
| d 4-14 | 1.01 | 1.03 | 0.057 | 0.835 |
| d 14-22 | 1.20 | 1.09 | 0.037 | 0.055 |
| d 4-22 | 1.10 | 1.04 | 0.036 | 0.268 |
| FCR ((g milk DM + g | 1.32 | 1.18 | 0.042 | 0.038 |
| creep feed DM)/g) | | | | |
| d 14-22 | | | | |

ADG = average daily gain; ADFI = average daily feed intake; SEM = standard error of mean; DM = dry matter.

Results—2′FL Promoted a Consortium of Beneficial Bacteria in Pig Gut, which Might Influence Feed Efficiency

Supplementation with 2′FL promoted several beneficial bacteria, namely F. Prausnitzii, L. mucosae and B. obeum, while inhibited Prevotella RS2. Table 6 shows the relative abundances (in %) of the species at the weaning day (day 22) that were significantly changed upon three-week supplementation of 2′FL (P<0.05). The changes in gut microbiota might be linked to the improved FCR during pre-weaning.

TABLE 6
Relative abundances of several species in fecal
samples of piglets at weaning day (d 22).

| Species | Control (%) | 2′FL (%) | P-value |
|---|---|---|---|
| Faecalibacterium prausnitzii | 0.023665 | 0.278013 | 0.00965 |
| Prevotella Sp. RS2 | 0.894466 | 0.109322 | 0.0129 |
| Lactobacillus mucosae | 0.028339 | 0.158584 | 0.0263 |
| Blautia obeum | 0.073608 | 0.167207 | 0.0428 |

F. prausnitzii has been consistently reported as one of the main butyrate producers found in the intestine was promoted in piglets with a better weight gain (Cheng et al., 2019, Animal 13(3): p. 533-541). Lactobacillus mucosae has been shown to improve feed efficiency upon challenge with Lipopolysaccharide (LPS) (Li et al., 2021, J. Anim. Sci. 199(12): skab286; Chiang et al., 2015, Asian-Australas J. Anim. Sci. 28(8): p. 1163-1170). Blautia are among beneficial bacteria found in pigs' gut and other mammals' gut. B. obeum (previously known as Ruminococcus obeum) is positively associated with feed efficiency (Bergamaschi et al., 2020, Microbiome 8(1): 110) in pigs. Several studies indicate that the abundance of Prevotella spp. is inversely correlated with feed efficiency in pigs (Tan et al., 2017, Front. Microbiol. 8:1546; Quan et al., 2018, Sci. Rep. 8(1): 4536; Yang et al., 2017, Front. Microbiol. 8:502).

1.-22. (canceled)

23. A method of reducing the feed conversion ratio (FCR) of a farm animal, the method comprising administering to the farm animal an effective amount of:
(i) a fucosylated mammalian milk oligosaccharide (MMO),
(ii) a sialylated MMO, or
(iii) a sialylated MMO and a fucosylated MMO,
thereby reducing the FCR of the farm animal.

24. The method according to claim 23, wherein a sialylated MMO and a fucosylated MMO are administered to the farm animal.

25. The method according to claim 24, wherein administration of a combination of a sialylated MMO and a fucosylated MMO results in a synergistic reduction in FCR relative to administration of either MMO alone.

26. The method according to claim 23, wherein administration of the fucosylated MMO and/or the administered sialylated MMO reduces FCR compared with farm animals not receiving the MMO.

27. The method according to claim 23, wherein administration of the MMO increases the average daily weight gain of the farm animal compared with a control animal not receiving the MMO.

28. The method according to claim 23, wherein the farm animal is selected from the group consisting of bovine, pig, sheep, goat, horse, donkey, mule, hinny, camelid, llama, alpaca, rabbit, fish, crustacean, and poultry.

29. The method according to claim 23, wherein the farm animal is administered a fucosylated MMO comprising a fucose that is linked to a galactose in an α-1,2-linkage.

30. The method according to claim 29, wherein the fucosylated MMO is 2′-fucosyllactose.

31. The method according to claim 29, wherein the fucosylated MMO is part of a composition.

32. The method according to claim 23, wherein the farm animal is administered a sialylated MMO comprising a sialic acid or a N-acetylneuraminic acid (Neu5Ac) that is linked to a monosaccharide in an α-2,3- or an α-2,6-linkage, and wherein the monosaccharide is galactose or N-acetylglucosamine.

33. The method according to claim 32, wherein the sialylated MMO is 3′-sialyllactose (3′SL) or 6′-sialyllactose (6′SL).

34. The method according to claim 32, wherein the sialylated MMO is part of a composition.

35. The method according to claim 23, wherein the fucosylated MMO is administered at a dosage of 0.1 g to 10 g per kg of feed and/or wherein the sialylated MMO is administered at a dosage of 0.1 to 10 g per kg of feed.

36. The method according to claim 23, wherein fucosylated MMO and sialylated MMO are administered to the farm animal in two different stages, wherein stage 1 is administering the sialylated MMO and optionally administering the fucosylated MMO; and stage 2 is administering the fucosylated MMO, optionally without administering the sialylated MMO.

37. The method according to claim 23, wherein fucosylated MMO and sialylated MMO are administered to the farm animal in two different stages, wherein stage 1 is administering the sialylated MMO and the fucosylated MMO; and stage 2 is administering the fucosylated MMO, without administering the sialylated MMO.

38. The method according to claim 23, wherein the sialylated MMO is a first sialylated MMO and a second sialylated MMO and optionally a further sialylated MMO.

39. The method according to claim 38, wherein the first sialylated MMO comprises a sialic acid or a N-acetylneuraminic acid (Neu5Ac) that is linked to a monosaccharide in an α-2,3-linkage, and wherein the monosaccharide is galactose or N-acetylglucosamine.

40. The method according to claim 38, wherein the first sialylated MMO is 3′-sialyllactose (3′SL).

41. The method according to claim 38, wherein the second sialylated MMO comprises a sialic acid or a N-acetylneuraminic acid (Neu5Ac) that is linked to a monosaccharide in an α-2,6-linkage, and wherein the monosaccharide is galactose or N-acetylglucosamine.

42. The method according to claim 38, wherein the second sialylated MMO is 6′-sialyllactose (6′SL).

43. A method of increasing the body weight of a farm animal, the method comprising administering to the farm animal an effective amount of a sialylated MMO and a fucosylated MMO, thereby increasing the body weight of the farm animal.

44. The method according to claim 43, wherein the fucosylated MMO and the sialylated MMO are administered to the farm animal in two different stages, wherein stage 1 is administering the sialylated MMO and optionally administering the fucosylated MMO; and stage 2 is administering the fucosylated MMO, optionally without administering the sialylated MMO.

45. The method according to claim 43, wherein the fucosylated MMO and the sialylated MMO are administered to the farm animal in two different stages, wherein stage 1 is administering the sialylated MMO and the fucosylated MMO; and stage 2 is administering the fucosylated MMO without administering the sialylated MMO.

46. The method according to claim 43, wherein the fucosylated MMO comprises a fucose that is linked to a galactose in an α-1,2-linkage.

47. The method according to claim 43, wherein the sialylated MMO comprises a sialic acid or a N-acetylneuraminic acid (Neu5Ac) that is linked to a monosaccharide in an α-2,3- or an α-2,6-linkage, and wherein the monosaccharide is galactose or N-acetylglucosamine.
